SEQUENCE LISTING (39 sequences)

SEQ ID NO: 1 - protein, 119 aa, synthetic construct
QVQLQESGPGLVKPSETLSLTCSVSGGSINSYYWTWIRQPPGKGLEWVGYIYHSGSTSYNPSLKSRITISVAPSKNHFSLELTSMTAADTAVYYCARLGGHGDYGSDYWGQGTLVTVSS

SEQ ID NO: 2 - protein, 112 aa, synthetic construct
DIVMTQSPVSLPVTPGEPASISCNSSQSLLHSNGYAHLDWYLQKPGQSPKLMIYLGLNRAFGVPDRFSGSGSGTDFTLKISRVEAEDVGVYYCMQALQTPFTFGPGTRVDIK

SEQ ID NO: 3 - protein, 119 aa, synthetic construct
QVQLQGSGPGLLRPSETLSLTCSVSGVSINSYYWSWVRQPPGKALEWIGYIYYSGNTNYNPSLESRVTISVDRSKNQFSLKMTSVTAADTARYFCARQGIFGDYGSDYWGPGTLVTVSS

SEQ ID NO: 4 - protein, 112 aa, synthetic construct
DIVMTQSPLSLPVTPGEPASISCRSNQSLQHSNGYVHLDWYRQKPGQSPHLLIYLGFNRASGVPDRFSGGGSGTDFTLKISRVEAEDVGVYYCMQGLQTPFTFGPGTTVDLK

SEQ ID NO: 5 - protein, 123 aa, synthetic construct
QVQLVQSGSELKRPGASVKVSCRASGYTFTSYTINWVRQAPGQGLEWMGWINTSTGDPTYAQGFTGRFVFSLDTSVSTAYLEISRLKAEDTAVYYCARAFGLTVVRGGIVGVWGQGTTVTVSS

SEQ ID NO: 6 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYSQTFGQGTKVEIK

SEQ ID NO: 7 - protein, 123 aa, synthetic construct
QVQLQESGPGLVKPSETLSLTCTVSGGSVRSASYAWSWIRQPPGKGLEWIGDIYYSGTTNYNPSLKSRVTLSVDTAKNRFSLRLRSVTAADTAVYHCARERYYYGSSGDFDYWGQGTLVTVSS

SEQ ID NO: 8 - protein, 107 aa, synthetic construct
DIQMTQSPSSLSASVGDRVTITCRASQGIRNYLAWFQQKPGQAPKSLIFAASSLHTGVPSRFSGSGSGTDFTLTISSLQPEDFATYYCQHYNSYPPTFGQGTKLEIK

SEQ ID NO: 9 - DNA, 357 bp, synthetic construct
  source 1..357: mol_type = other DNA
caggtgcagctgcaggagtcgggccccggactggtgaagccttcggagaccctgtccctcacctgcagtgtctctggtggctccatcaatagttactactggacctggattcggcagcccccggggaagggactggagtgggttggctatatttatcacagtggaagcaccagctacaacccctccctcaagagtcgaatcaccatttccgtagccccgtccaagaaccacttctccctggagctgacctctatgaccgctgcagacacggccgtctattactgtgcgagactggggggccacggtgactacggttccgactactggggccagggaaccctggtcaccgtctcctca

SEQ ID NO: 10 - DNA, 336 bp, synthetic construct
  source 1..336: mol_type = other DNA
gatattgtgatgactcagtctccagtctccctgcccgtcactcctggagagccggcctccatctcctgcaactctagtcagagcctcctgcatagcaatggatacgcccatttggattggtacctgcagaagccagggcagtctccaaagctcatgatctacctgggtcttaatcgggccttcggggtccctgacaggttcagtggcagtggatcaggcacagattttacactgaaaatcagcagagtggaggctgaggatgttggcgtttattactgcatgcaagctctacaaactccattcactttcggccctgggaccagagtggatatcaaa

SEQ ID NO: 11 - DNA, 357 bp, synthetic construct
  source 1..357: mol_type = other DNA
caggtgcagctgcaagggtcgggcccaggactgctgaggccttcggagaccctgtccctcacctgctctgtctccggtgtctccatcaatagttactattggagttgggtccggcagcccccggggaaggcacttgagtggattggctatatctattatagtggcaacaccaactacaatccctccctcgagagtcgagtcaccatatcagtggacaggtccaagaaccagttctccctgaagatgacctctgtgaccgctgcggatacggccagatatttctgtgcgagacaggggatcttcggtgactacggctccgactactggggcccgggaaccctggtcaccgtctcctca

SEQ ID NO: 12 - DNA, 336 bp, synthetic construct
  source 1..336: mol_type = other DNA
gatattgtgatgactcagtctccactctccctgcccgtcacccctggagagccggcctccatctcctgtaggtctaatcagagcctccagcatagtaatggatacgtccatttggattggtacaggcagaagccagggcagtctccacacctcctgatctacttgggctttaatcgggcctccggggtccctgacaggttcagtggcggtggatcaggcacagattttacactgaaaatcagcagagtggaggctgaggatgttggagtttattattgcatgcaaggtctacaaactccattcactttcggccctgggaccacagtggatctcaaa

SEQ ID NO: 13 - DNA, 369 bp, synthetic construct
  source 1..369: mol_type = other DNA
caagtgcagctggtgcaatctgggtctgagttgaagaggcctggggcctcagtgaaggtttcctgtagggcttctgggtacaccttcactagttatactatcaactgggtgcgacaggcccctggacaagggcttgagtggatgggatggatcaacaccagcactggggacccaacatatgcccagggcttcacaggacggtttgtcttctccttggacacctctgtcagcacggcatatctggagatcagcaggctaaaggctgaagacactgccgtgtattactgtgcgagagccttcggccttactgtggttcggggaggtattgtcggcgtctggggccaagggaccacggtcaccgtctcctca

SEQ ID NO: 14 - DNA, 321 bp, synthetic construct
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattctcagacgttcggccaagggaccaaggtggaaatcaaa

SEQ ID NO: 15 - DNA, 369 bp, synthetic construct
  source 1..369: mol_type = other DNA
caggtgcagctgcaggagtcgggcccaggactggtgaagccttcggagaccctgtccctcacttgcactgtgtctggtggctccgtcagaagtgctagttacgcctggagctggatccggcagccccctgggaaaggactggagtggattggcgatatctattacagtgggaccaccaattacaacccgtccctcaagagtcgagtcaccctatcggtcgacacggccaagaaccggttctccctgaggctgaggtctgtgaccgctgcggacacggccgtgtatcactgtgcgagagagaggtattactatggtagtagtggtgactttgactactggggccagggaaccctggtcaccgtctcctca

SEQ ID NO: 16 - DNA, 321 bp, synthetic construct
  source 1..321: mol_type = other DNA
gacatccagatgacccagtctccatcctcactgtctgcatctgtaggcgacagagtcaccatcacttgtcgggcaagtcagggcattagaaattatttagcctggtttcagcagaaaccagggcaagcccctaagtccctgatctttgctgcatccagtttgcacactggggtcccatcgaggttcagcggcagtggatctgggacagatttcactctcaccatcagcagcctgcagcctgaggattttgcaacatattactgccaacactataatagttaccctcccacttttggccaggggaccaagctggagatcaaa

SEQ ID NO: 17 - protein, 112 aa, synthetic construct
DIVMTQSPVSLPVTPGEPASISCNSAQSLLHSNGYAHLDWYLQKPGQSPKLMIYLGLNRAFGVPDRFSGSGSGTDFTLKISRVEAEDVGVYYCMQALQTPFTFGPGTRVDIK

SEQ ID NO: 18 - protein, 112 aa, synthetic construct
DIVMTQSPLSLPVTPGEPASISCRSNQALQHSNGYVHLDWYRQKPGQSPHLLIYLGFNRASGVPDRFSGGGSGTDFTLKISRVEAEDVGVYYCMQGLQTPFTFGPGTTVDLK

SEQ ID NO: 19 - protein, 112 aa, synthetic construct
DIVMTQSPVSLPVTPGEPASISCQSSQSLLHSNGYAHLDWYLQKPGQSPKLMIYLGLNRAFGVPDRFSGSGSGTDFTLKISRVEAEDVGVYYCMQALQTPFTFGPGTRVDIK

SEQ ID NO: 20 - protein, 112 aa, synthetic construct
DIVMTQSPLSLPVTPGEPASISCRSQQSLQHSNGYVHLDWYRQKPGQSPHLLIYLGFNRASGVPDRFSGGGSGTDFTLKISRVEAEDVGVYYCMQGLQTPFTFGPGTTVDLK

SEQ ID NO: 21 - protein, 112 aa, synthetic construct
DIVMTQSPVSLPVTPGEPASISCRSSQSLLHSNGYAHLDWYLQKPGQSPKLMIYLGLNRAFGVPDRFSGSGSGTDFTLKISRVEAEDVGVYYCMQALQTPFTFGPGTRVDIK

SEQ ID NO: 22 - protein, 112 aa, synthetic construct
DIVMTQSPLSLPVTPGEPASISCRSSQSLQHSNGYVHLDWYRQKPGQSPHLLIYLGFNRASGVPDRFSGGGSGTDFTLKISRVEAEDVGVYYCMQGLQTPFTFGPGTTVDLK

SEQ ID NO: 23 - protein, 123 aa, synthetic construct
QVQLVQSGSELKRPGASVKVSCRASGYTFTSYTINWVRQAPGQGLEWMGWINTATGDPTYAQGFTGRFVFSLDTSVSTAYLEISRLKAEDTAVYYCARAFGLTVVRGGIVGVWGQGTTVTVSS

SEQ ID NO: 24 - protein, 6 aa, synthetic construct
HHHHHH

SEQ ID NO: 25 - protein, 95 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYC

SEQ ID NO: 26 - protein, 96 aa, synthetic construct
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYC

SEQ ID NO: 27 - protein, 97 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYC

SEQ ID NO: 28 - protein, 93 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYC

SEQ ID NO: 29 - protein, 88 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYC

SEQ ID NO: 30 - protein, 88 aa, synthetic construct
DIQMTQSPSSLSASVGDRVTITCRASQGISNYLAWFQQKPGKAPKSLIYAASSLQSGVPSRFSGSGSGTDFTLTISSLQPEDFATYYC

SEQ ID NO: 31 - protein, 329 aa, synthetic construct
QDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTR

SEQ ID NO: 32 - protein, 320 aa, synthetic construct
DKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRANGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISSLPFQNIDSRAVGKCPRYVKQRSLLLATGMKNVPEIPKG

SEQ ID NO: 33 - protein, 317 aa, synthetic construct
ATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTTNGGSNACKRPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRGKSSIMRSDAPIDTCISECITPNGSIPNNKPFQNVNKITYGACPKYVKQNTLKLATGMRNVPEKQTR

SEQ ID NO: 34 - protein, 317 aa, synthetic construct
ATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTTNGGSNACKRPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRGKSSIMRSDAPIDTCISECHTPNGSIPNNKPFQNVNKITYGACPKYVKQNTLKLATGMRNVPEKQTR

SEQ ID NO: 35 - protein, 317 aa, synthetic construct; identical to SEQ ID NO: 33

SEQ ID NO: 36 - protein, 317 aa, synthetic construct; identical to SEQ ID NO: 33

SEQ ID NO: 37 - protein, 317 aa, synthetic construct
ATLCLGHHAVPNGTLVKTITDDQIEVTNATELVQSSSVGKICNNPHRILDGIDCTLIDALLGDPHCDVFQNETWDLFVERSKAFSNCYPYDVPDYASLRSLVASSGTLEFITEGFTWTGVTTNGTSNACKRPGSGFFSRLNWLTKSGSTYPVLNVTMPNNDNDDKLYIWGIHHPGTNTEQTSLYVQASGRITVSTRRSQQTIIPNIGSRPWVRGQSSRISIYWTIVKPGDVLVINSNGNLIAPRGYFKMRGKSSIMRSEAPIDTCISECITPNGSIPNNKPFQNVNKITYGACPKYVKQETLKLATGMRNVPEKQTR

SEQ ID NO: 38 - protein, 317 aa, synthetic construct
ATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFIPEGFTWNGVTTNGVSNACKRPGSGFFSRLNWLTKSGSTYPVLNVTMPNNKEFDKIYIWGIHHPSTNQEQTSLYVQASGRVTVSTRRSQQTIIPNIGSRPWVRGQSSRISIYWTIVKPGDVLVINSNGNLIAPRGYFKMRGKSSIMRSRAPIDTCISECITPNGSIPNNKPFQNVNKITYGACPKYVKQNTLKLATGMRNVPEVQTR

SEQ ID NO: 39 - protein, 317 aa, synthetic construct
ATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWNGVTTNGVSNACKRPGSGFFSRLNWLTKSGSTYPVLNVTMPNNDNFDKIYIWGIHHPSTNQEQTSLYVQASGRVTVSTRRSQQTIIPNIGSRPWVRGLSSRISIYWTIVKPGDVLVINSNGNLIAPRGYFKMRGKSSIMRSDAPIDTCISECITPNGSIPNNKPFQNVNKITYGACPKYVKQNTLKLATGMRNVPEKQTR

(19) United States
(12) Patent Application Publication
(10) Pub. No.: US 2026/0174891 A1
(43) Pub. Date: Jun. 25, 2026

(54) N-LINKED GLYCAN REMOVAL MUTANT ANTIBODIES AND THEIR USE AGAINST INFLUENZA VIRUSES

(71) Applicants: The Trustees of Columbia University in the City of New York, New York, NY (US); The Rockefeller University, New York, NY (US)

(72) Inventors: Xueling WU, Potomac, MD (US); Manxue JIA, Sugar Land, TX (US); David D. HO, Bedford, NY (US); Lawrence SHAPIRO, New York, NY (US); Nicholas C. MORANO, New York, NY (US); Hong LU, Oakland Gardens, NY (US)

(21) Appl. No.: 19/548,500

(22) Filed: Feb. 24, 2026

Related U.S. Application Data
(63) Continuation of application No. PCT/US24/43459, filed on Aug. 22, 2024.
(60) Provisional application No. 63/578,505, filed on Aug. 24, 2023.
(60) Provisional application No. 63/597,463, filed on Nov. 9, 2023.
(60) Provisional application No. 63/650,342, filed on May 21, 2024.

(51) Int. Cl. A61K 47/68

(52) U.S. Cl. CPC A61K 47/6849; C07K 2317/24; C07K 2317/55; C07K 2317/56; C07K 2317/76

(57) ABSTRACT

Disclosed herein are vectors, synthetic antibodies, and methods of producing and using the same, comprising at least one polynucleotide sequence encoding an antibody or fragment thereof, wherein a variable heavy chain domain of the antibody or fragment thereof comprises SEQ ID NO: 1, SEQ ID NO: 3, SEQ ID NO: 5, or SEQ ID NO: 7, and a variable light chain domain of the antibody or fragment thereof comprises SEQ ID NO: 17, SEQ ID NO: 18, SEQ ID NO: 19, SEQ ID NO: 20, SEQ ID NO: 21, or SEQ ID NO: 22; or wherein a variable heavy chain domain of the antibody or fragment thereof comprises SEQ ID NO: 23, and a variable light chain domain of the antibody or fragment thereof comprises SEQ ID NO: 2, SEQ ID NO: 4, SEQ ID NO: 6, or SEQ ID NO: 8.

This application is a continuation of International Patent Application No. PCT/US2024/043459, filed on Aug. 22, 2024, which claims the benefit of and priority to U.S. Provisional Ser. No. 63/578,505, filed Aug. 24, 2023; U.S. Provisional Ser. No. 63/597,463, filed Nov. 9, 2023; and U.S. Provisional Ser. No. 63/650,342, filed May 21, 2024, the contents of each of which are hereby incorporated by reference in their entireties.

### GOVERNMENT SUPPORT

This invention was made with government support under W911NF-14-C-0001 awarded by the Department of Defense. The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entireties.

### SEQUENCE LISTING

A sequence listing, which is part of the present disclosure, is submitted herewith in electronic format (filename: 0019240_01321US5_SL.xml, created on Feb. 23, 2026, size: 42,293 bytes). The information in the electronic sequence listing is hereby incorporated by reference in its entirety.

### TECHNICAL FIELD

The present invention relates generally to antibodies and expression systems for producing antibodies. More particularly, the present invention relates to antibodies and expression systems for producing antibodies for use in prevention and treatment against influenza viruses.

### BACKGROUND

There is currently no prevention or treatment specifically to H7N9 or emerging influenza viruses.

### SUMMARY

In one aspect, described herein is a vector comprising at least one polynucleotide sequence encoding an antibody or fragment thereof, wherein the antibody or fragment thereof comprises a variable heavy chain domain and a variable light chain domain, wherein the variable heavy chain domain of the antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23 and the variable light chain domain of the antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the variable heavy chain domain of the antibody or fragment thereof comprises the amino acid sequence of SEQ ID NO: 1, and the variable light chain domain of the antibody or fragment thereof comprises the amino acid sequence of SEQ ID NO: 17. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 19. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 21. In some embodiments, the variable heavy chain domain of the antibody or fragment thereof comprises the amino acid sequence of SEQ ID NO: 3, and the variable light chain domain of the antibody or fragment thereof comprises the amino acid sequence of SEQ ID NO: 18. In some embodiments, the variable heavy chain domain of the antibody or fragment thereof is the amino acid sequence of SEQ ID NO: 3, and the variable light chain domain of the antibody or fragment thereof is the amino acid sequence of SEQ ID NO: 20. In some embodiments, the variable heavy chain domain of the antibody or fragment thereof comprises the amino acid sequence of SEQ ID NO: 3, and the variable light chain domain of the antibody or fragment thereof comprises the amino acid sequence of SEQ ID NO: 22. In some embodiments, the variable heavy chain domain of the antibody or fragment thereof is the amino acid sequence of SEQ ID NO: 23, and the variable light chain domain of the antibody or fragment thereof is the amino acid sequence of SEQ ID NO: 6. In some embodiments, the antibody or fragment thereof is configured to bind to a hemagglutinin (HA) protein of an H7N9 influenza virus. In some embodiments, the antibody or fragment thereof is configured to neutralize the H7N9 influenza virus. In some embodiments, the antibody or fragment thereof is configured to bind to a globular head domain (HA1) of a hemagglutinin (HA) protein of an H7N9 influenza virus. In some embodiments, the antibody or fragment thereof is configured to bind to an overlapping region of an antigenic site D of the globular head domain (HA1). In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding. In some embodiments, the antibody or fragment thereof is configured to bind to a stalk region (HA2) of a hemagglutinin (HA) protein of an H7N9 influenza virus. In some embodiments, described herein is a host cell comprising a vector described herein.

In another aspect, described herein is a method of making an antibody or fragment thereof comprising: culturing a host cell under conditions suitable for an expression of a vector, wherein the vector comprises at least one polynucleotide sequence encoding the antibody or fragment thereof, wherein the antibody or fragment thereof comprises a variable heavy chain domain and a variable light chain domain, wherein the variable heavy chain domain of the antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22; and recovering the antibody or fragment thereof. In some embodiments, the variable heavy chain domain of the antibody or fragment thereof comprises the amino acid sequence of SEQ ID NO: 1, and the variable light chain domain of the antibody or fragment thereof comprises the amino acid sequence of SEQ ID NO: 17. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 19. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 21. In some embodiments, the variable heavy chain domain of the antibody or fragment thereof comprises the amino acid sequence of SEQ ID NO: 3, and the variable light chain domain of the antibody or fragment thereof comprises the amino acid sequence of SEQ ID NO: 18. In some embodiments, the variable heavy chain domain of the antibody or fragment thereof is the amino acid sequence of SEQ ID NO: 3, and the variable light chain domain of the antibody or fragment thereof is the amino acid sequence of SEQ ID NO: 20. In some embodiments, the variable heavy chain domain of the antibody or fragment thereof is the amino acid sequence of SEQ ID NO: 3, and the variable light chain domain of the antibody or fragment thereof is the amino acid sequence of SEQ ID NO: 22. In some embodiments, the variable heavy chain domain of the antibody or fragment thereof comprises the amino acid sequence of SEQ ID NO: 23, and the variable light chain domain of the antibody or fragment thereof comprises the amino acid sequence of SEQ ID NO: 6. In some embodiments, the antibody or fragment thereof is configured to bind to a hemagglutinin (HA) protein of an H7N9 influenza virus. In some embodiments, the antibody or fragment thereof is configured to neutralize the H7N9 virus. In some embodiments, the antibody or fragment thereof is configured to bind to a globular head domain (HA1) of a hemagglutinin (HA) protein of an H7N9 virus. In some embodiments, the antibody or fragment thereof is configured to bind to a portion overlapping with an antigenic site D of the globular head domain (HA1). In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a loop of the HA protein required for sialic acid binding. In some embodiments, the antibody or fragment thereof is configured to bind to a stalk region (HA2) of a hemagglutinin (HA) protein of an H7N9 influenza virus.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the synthetic antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the variable heavy chain domain of the synthetic antibody or fragment thereof comprises the amino acid sequence of SEQ ID NO: 1, and the variable light chain domain of the synthetic antibody or fragment thereof comprises the amino acid sequence of SEQ ID NO: 17. In some embodiments, the variable heavy chain domain of the synthetic antibody or fragment thereof is the amino acid sequence of SEQ ID NO: 1, and the variable light chain domain of the synthetic antibody or fragment thereof is the amino acid sequence of SEQ ID NO: 19. In some embodiments, the variable heavy chain domain of the synthetic antibody or fragment thereof is the amino acid sequence of SEQ ID NO: 1, and the variable light chain domain of the synthetic antibody or fragment thereof is the amino acid sequence of SEQ ID NO: 21. In some embodiments, the variable heavy chain domain of the synthetic antibody or fragment thereof comprises the amino acid sequence of SEQ ID NO: 3, and the variable light chain domain of the synthetic antibody or fragment thereof comprises the amino acid sequence of SEQ ID NO: 18. In some embodiments, the variable heavy chain domain of the synthetic antibody or fragment thereof is the amino acid sequence of SEQ ID NO: 3, and the variable light chain domain of the synthetic antibody or fragment thereof is the amino acid sequence of SEQ ID NO: 20. In some embodiments, the variable heavy chain domain of the synthetic antibody or fragment thereof is the amino acid sequence of SEQ ID NO: 3, and the variable light chain domain of the synthetic antibody or fragment thereof is the amino acid sequence of SEQ ID NO: 22. In some embodiments, the variable heavy chain domain of the synthetic antibody or fragment thereof comprises the amino acid sequence of SEQ ID NO: 23, and the variable light chain domain of the synthetic antibody or fragment thereof comprises the amino acid sequence of SEQ ID NO: 6. In some embodiments, the antibody or fragment thereof is configured to bind to a hemagglutinin (HA) protein of an H7N9 influenza virus. In some embodiments, the antibody or fragment thereof is configured to bind to a portion overlapping with an antigenic site D of the globular head domain (HA1). In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a loop of the HA protein required for sialic acid binding. In some embodiments, the antibody or fragment thereof is configured to bind to a stalk region (HA2) of a hemagglutinin (HA) protein of an H7N9 virus.

In another aspect, described herein is a method of preventing or treating an infection in a subject caused by an H7N9 influenza virus, the method comprising administering to the subject an effective amount of a pharmaceutical composition, wherein the pharmaceutical composition comprises: a synthetic antibody or fragment thereof comprising a variable heavy chain domain and a variable light chain domain, wherein the variable heavy chain domain of the synthetic antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the synthetic antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the variable heavy chain domain of the first antibody or fragment thereof comprises the amino acid sequence of SEQ ID NO: 1, and the variable light chain domain of the first antibody or fragment thereof comprises the amino acid sequence of SEQ ID NO: 17. In some embodiments, the variable heavy chain domain of the first antibody or fragment thereof is the amino acid sequence of SEQ ID NO: 1, and the variable light chain domain of the first antibody or fragment thereof is the amino acid sequence of SEQ ID NO: 19. In some embodiments, the variable heavy chain domain of the first antibody or fragment thereof is the amino acid sequence of SEQ ID NO: 1, and the variable light chain domain of the first antibody or fragment thereof is the amino acid sequence of SEQ ID NO: 21. In some embodiments, the variable heavy chain domain of the first antibody or fragment thereof comprises the amino acid sequence of SEQ ID NO: 3, and the variable light chain domain of the first antibody or fragment thereof comprises the amino acid sequence of SEQ ID NO: 18. In some embodiments, the variable heavy chain domain of the first antibody or fragment thereof is the amino acid sequence of SEQ ID NO: 3, and the variable light chain domain of the first antibody or fragment thereof is the amino acid sequence of SEQ ID NO: 20. In some embodiments, the variable heavy chain domain of the first antibody or fragment thereof is the amino acid sequence of SEQ ID NO: 3, and the variable light chain domain of the first antibody or fragment thereof is the amino acid sequence of SEQ ID NO: 22. In some embodiments, the variable heavy chain domain of the first antibody or fragment thereof comprises the amino acid sequence of SEQ ID NO: 23, and the variable light chain domain of the first antibody or fragment thereof comprises the amino acid sequence of SEQ ID NO: 6. In some embodiments, the method comprises administering to the subject the effective amount of the pharmaceutical composition to prevent the infection in the subject caused by an H7N9 influenza virus. In some embodiments, the effective amount of the pharmaceutical composition is in the range of about 0.1 mg/kg body weight to about 0.5 mg/kg body weight per dose. In some embodiments, at least one of (i) the first antibody or fragment thereof or (ii) second antibody or fragment thereof is configured to bind to a hemagglutinin (HA) protein of an H7N9 influenza virus. In some embodiments, at least one of (i) the first antibody or fragment thereof or (ii) second antibody or fragment thereof is configured to neutralize the H7N9 virus. In some embodiments, the first antibody or fragment thereof is configured to bind to a globular head domain (HA1) of a hemagglutinin (HA) protein of an H7N9 virus. In some embodiments, the first antibody or fragment thereof is configured to bind to a portion overlapping with an antigenic site D of the globular head domain (HA1). In some embodiments, the first antibody or fragment thereof is configured to bind to a β14-centered surface of a lateral patch of the globular head domain (HA1) of the H7N9 influenza virus. In some embodiments, the first antibody or fragment thereof is configured to disrupt a 220-loop of the HA protein required for sialic acid binding. In some embodiments, the second antibody or fragment thereof is configured to bind to a stalk region (HA2) of a hemagglutinin (HA) protein of an H7N9 influenza virus.

In another aspect, described herein is a means for binding a hemagglutinin (HA) protein of an H7N9 virus. In some embodiments, the means comprises a variable heavy chain domain of an antibody or fragment thereof comprising an amino acid sequence selected from the group consisting of SEQ ID NO: 1, SEQ ID NO: 3, SEQ ID NO: 5, SEQ ID NO: 7, and SEQ ID NO: 23, and a variable light chain domain of the antibody or fragment thereof comprising the amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the means comprises a variable heavy chain domain of an antibody or fragment thereof comprising the amino acid sequence of SEQ ID NO: 1, and a variable light chain domain of the antibody or fragment thereof comprising the amino acid sequence of SEQ ID NO: 17. In some embodiments, the means are a variable heavy chain domain of an antibody or fragment thereof consisting of the amino acid sequence of SEQ ID NO: 1, and a variable light chain domain of the antibody or fragment thereof consisting of the amino acid sequence of SEQ ID NO: 19. In some embodiments, the means are a variable heavy chain domain of an antibody or fragment thereof consisting of the amino acid sequence of SEQ ID NO: 1, and a variable light chain domain of the antibody or fragment thereof consisting of the amino acid sequence of SEQ ID NO: 21. In some embodiments, the means comprises a variable heavy chain domain of an antibody or fragment thereof comprising the amino acid sequence of SEQ ID NO: 3, and a variable light chain domain of the antibody or fragment thereof comprising the amino acid sequence of SEQ ID NO: 18. In some embodiments, the means consisting of a variable heavy chain domain of an antibody or fragment thereof comprising the amino acid sequence of SEQ ID NO: 3, and a variable light chain domain of the antibody or fragment thereof consisting of the amino acid sequence of SEQ ID NO: 20. In some embodiments, the means consisting of a variable heavy chain domain of an antibody or fragment thereof comprising the amino acid sequence of SEQ ID NO: 3, and a variable light chain domain of the antibody or fragment thereof consisting of the amino acid sequence of SEQ ID NO: 22. In some embodiments, the means comprises a variable heavy chain domain of an antibody or fragment thereof comprising the amino acid sequence of SEQ ID NO: 23, and a variable light chain domain of the antibody or fragment thereof comprising the amino acid sequence of SEQ ID NO: 6. In some embodiments, the means is configured to bind to a portion overlapping with an antigenic site D of a globular head domain (HA1) of the HA protein of the H7N9 virus. In some embodiments, the means is configured to bind to a β14-centered surface of a lateral patch of the globular head domain (HA1) of the H7N9 influenza virus. In some embodiments, the means is configured to disrupt a 220-loop of the HA protein required for sialic acid binding. In some embodiments, the means is configured to bind to a stalk region (HA2) of the HA protein of an H7N9 virus.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 1, SEQ ID NO: 3, SEQ ID NO: 5, SEQ ID NO: 7, or SEQ ID NO: 23, and the variable light chain domain of the synthetic antibody or fragment thereof comprises an amino acid sequence that is at least 80% identical to SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, or SEQ ID NO: 22. In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 17. In some embodiments, the variable heavy chain domain of the synthetic antibody or fragment thereof is the amino acid sequence that is at least 80% identical to SEQ ID NO: 1, and the variable light chain domain of the synthetic antibody or fragment thereof is the amino acid sequence that is at least 80% identical to SEQ ID NO: 19. In some embodiments, the variable heavy chain domain of the synthetic antibody or fragment thereof is the amino acid sequence that is at least 80% identical to SEQ ID NO: 1, and the variable light chain domain of the synthetic antibody or fragment thereof is the amino acid sequence that is at least 80% identical to SEQ ID NO: 21. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 18. In some embodiments, the variable heavy chain domain of the synthetic antibody or fragment thereof is the amino acid sequence that is at least 80% identical to SEQ ID NO: 3, and the variable light chain domain of the synthetic antibody or fragment thereof is the amino acid sequence that is at least 80% identical to SEQ ID NO: 20. In some embodiments, the variable heavy chain domain of the synthetic antibody or fragment thereof is the amino acid sequence that is at least 80% identical to SEQ ID NO: 3, and the variable light chain domain of the synthetic antibody or fragment thereof is the amino acid sequence that is at least 80% identical to SEQ ID NO: 22. In some embodiments, the variable heavy chain domain of the synthetic antibody or fragment thereof comprises the amino acid sequence that is at least 80% identical to SEQ ID NO: 23, and the variable light chain domain of the synthetic antibody or fragment thereof comprises the amino acid sequence that is at least 80% identical to SEQ ID NO: 6. In some embodiments, the synthetic antibody or fragment thereof is configured to bind to a hemagglutinin (HA) protein of an H7N9 influenza virus. In some embodiments, the synthetic antibody or fragment thereof is configured to bind to a portion overlapping with an antigenic site D of the globular head domain (HA1). In some embodiments, the synthetic antibody or fragment thereof is configured to bind to a β14-centered surface of a lateral patch of the globular head domain (HA1) of the H7N9 influenza virus. In some embodiments, the synthetic antibody or fragment thereof is configured to disrupt a 220-loop of the HA protein required for sialic acid binding. In some embodiments, the synthetic antibody or fragment thereof is configured to bind to a stalk region (HA2) of a hemagglutinin (HA) protein of an H7N9 virus. In some embodiments, a structure of the synthetic antibody or fragment thereof is substantially similar to a structure of a reference antibody or fragment thereof, wherein the reference antibody or structure thereof comprises a variable heavy chain domain and a variable light chain domain, wherein the variable heavy chain domain of the reference antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the reference antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the structure of the reference antibody or fragment thereof is determined by a structure prediction method. In some embodiments, the structure of the synthetic antibody or fragment thereof is determined by a structure prediction method. In some embodiments, the structure prediction method is AlphaFold or RaptorX. In some embodiments, the structure of the reference antibody or fragment thereof and the structure of the synthetic antibody or fragment thereof are further determined by X-ray crystallography or cryo-EM.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 1, SEQ ID NO: 3, SEQ ID NO: 5, SEQ ID NO: 7, and the variable light chain domain of the synthetic antibody or fragment thereof comprises an amino acid sequence that is at least 80% identical to SEQ ID NO: 17, SEQ ID NO: 18, SEQ ID NO: 19, SEQ ID NO: 20, SEQ ID NO: 21, or SEQ ID NO: 22.

In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 17. In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 19. In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 21. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 18. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 20. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 22. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding. In some embodiments, the synthetic antibody is configured to neutralize an H7N9 influenza virus.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 23 and the variable light chain domain of the synthetic antibody or fragment thereof comprises an amino acid sequence that is at least 80% identical to SEQ ID NO: 2, SEQ ID NO: 4, SEQ ID NO: 6, or SEQ ID NO: 8.

In some embodiments, the variable light chain domain of the synthetic antibody or fragment thereof comprises an amino acid sequence that is at least 80% identical to SEQ ID NO: 6. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding.

In another aspect, described herein is a synthetic antibody or fragment thereof comprising a variable heavy chain domain and a variable light chain domain, wherein the variable heavy chain domain of the synthetic antibody or fragment thereof comprises the complementarity-determining regions (CDRs) of SEQ ID NO: 1, SEQ ID NO: 3, SEQ ID NO: 5, or SEQ ID NO: 7 and the variable light chain domain of the synthetic antibody or fragment thereof comprises the CDRs of SEQ ID NO: 17, SEQ ID NO: 18, SEQ ID NO: 19, SEQ ID NO: 20, SEQ ID NO: 21, or SEQ ID NO: 22.

In some embodiments, the variable heavy chain domain of the synthetic antibody or fragment thereof comprises the CDRs of SEQ ID NO: 1, and the variable light chain domain of the synthetic antibody or fragment thereof comprises the CDRs of SEQ ID NO: 17. In some embodiments, the variable heavy chain domain of the synthetic antibody or fragment thereof comprises the CDRs of SEQ ID NO: 1, and the variable light chain domain of the synthetic antibody or fragment thereof comprises the CDRs of SEQ ID NO: 19. In some embodiments, the variable heavy chain domain of the synthetic antibody or fragment thereof comprises the CDRs of SEQ ID NO: 1, and the variable light chain domain of the synthetic antibody or fragment thereof comprises the CDRs of SEQ ID NO: 21. In some embodiments, the variable heavy chain domain of the synthetic antibody or fragment thereof comprises the CDRs of SEQ ID NO: 3, and the variable light chain domain of the synthetic antibody or fragment thereof comprises the CDRs of SEQ ID NO: 18. In some embodiments, the variable heavy chain domain of the synthetic antibody or fragment thereof comprises the CDRs of SEQ ID NO: 3, and the variable light chain domain of the synthetic antibody or fragment thereof comprises the CDRs of SEQ ID NO: 20. In some embodiments, the variable heavy chain domain of the synthetic antibody or fragment thereof comprises the CDRs of SEQ ID NO: 3, and the variable light chain domain of the synthetic antibody or fragment thereof comprises the CDRs of SEQ ID NO: 22. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding. In some embodiments, the synthetic antibody is configured to neutralize an H7N9 influenza virus.

In another aspect, described herein is a synthetic antibody or fragment thereof comprising a variable heavy chain domain and a variable light chain domain, wherein the variable heavy chain domain of the synthetic antibody or fragment thereof comprises the CDRs of SEQ ID NO: 23 and the variable light chain domain of the synthetic antibody or fragment thereof comprises the CDRs of SEQ ID NO: 2, SEQ ID NO: 4, SEQ ID NO: 6, or SEQ ID NO: 8.

In some embodiments, the variable light chain domain of the synthetic antibody or fragment thereof comprises the CDRs of SEQ ID NO: 6. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding.

In another aspect, described herein is a polynucleotide encoding a synthetic antibody described herein.

In another aspect, described herein is a method of preventing or treating an infection in a subject caused by an H7N9 influenza virus, the method comprising administering to the subject an effective amount of a pharmaceutical composition, wherein the pharmaceutical composition comprises a synthetic antibody described herein.

In another aspect, described herein is a method of preventing or treating an infection in a subject caused by an H7N9 influenza virus, the method comprising administering to the subject an effective amount of a pharmaceutical composition, wherein the pharmaceutical composition comprises a polynucleotide described herein.

### Definitions

As used herein, the term “antibody” includes synthetic antibodies, monoclonal antibodies, oligoclonal or polyclonal antibodies, multiclonal antibodies, recombinantly produced antibodies, intrabodies, multispecific antibodies, bispecific antibodies, monovalent antibodies, multivalent antibodies, human antibodies, humanized antibodies, chimeric antibodies, CDR-grafted antibodies, primatized antibodies, Fab fragments, F(ab′) fragments, F(ab′)₂ fragments, Fv fragments, single-chain FvFcs (scFvFc), single-chain Fvs (scFv), Dabs, nanobodies, anti-idiotypic (anti-Id) antibodies, and any other immunologically-reactive/antigen-binding fragments thereof. The term “immunoglobulin” (Ig) is used interchangeably with “antibody” herein.

The term “monoclonal antibody” as used herein refers to an antibody obtained from a population of substantially homogeneous antibodies, i.e., the individual antibodies comprising the population are substantially identical except for possible naturally occurring mutations and/or post-translation modifications (e.g., isomerizations, amidations) that may be present in some amounts. In some embodiments, monoclonal antibodies are highly specific, being directed against a single antigenic site. In contrast to polyclonal antibody preparations which typically include different antibodies directed against different determinants (epitopes), in some embodiments, each monoclonal antibody is directed against a single determinant on the antigen.

### BRIEF DESCRIPTION OF THE DRAWINGS

The following figures depict illustrative embodiments of the invention. The patent or application file contains at least one drawing executed in color. Copies of this patent or patent application publication with color drawing(s) will be provided by the Office upon request and payment of the necessary fee.

FIGS. 1A-1B show FACS depicting the staining and selection of H7-specific B cells from donor H7N9.HK2013 PBMCs. SSC-A, side scatter area; FSC-A, forward scatter area.

FIG. 2 shows single B-cell RT-PCR results. The left DNA gel shows PCR amplicons of IgG heavy chain variable region. The right DNA gel shows PCR amplicons of kappa chain variable region.

FIG. 3 depicts isolation and characterization of human H7N9 mAbs in vitro. FIG. 3 depicts ELISA binding curves of the indicated mAbs to soluble recombinant H7N9 HA, with and without Endo H treatment, to H7N9 HA1 from 2013, 2016, and 2017, to H7N7 HA, and to 6 non-H7 HA or HA1.

FIGS. 4A-4B depicts isolation and characterization of human H7N9 mAbs in vitro. FIG. 4A depicts a Western blot of a cleaved H7 HA (molecular mass of 43 kDa for HA1 and 30 kDa for HA2) with mAb H7.HK2 or H7.HK4. FIG. 4B shows neutralization curves of H7.HK mAbs against H7N9 2013 (left) and 2016 (right) pseudo viruses in MDCK cells. Data shown are mean±SEM.

FIGS. 5A-5B depicts comparisons of human H7N9 mAbs in vitro. FIG. 5A shows ELISA binding curves of the indicated mAbs to H7N9 HA1 from A/Shanghai/2/2013, A/Guangdong/17SF003/2016, and A/Hong Kong/125/2017. FIG. 5B shows ELISA binding curves of the indicated mAbs to H7N9 HA based on A/Shanghai/2/2013 and H7N7 HA based on A/Netherlands/219/2003.

FIG. 6 shows neutralization curves of the indicated mAbs against the 2013 and 2017 H7N9 pseudo viruses in MDCK cells. Data shown are mean±SEM. Similar results have been independently reproduced at least once.

FIG. 7 shows neutralization IC50 of H7.HK mAbs against pseudo virus or live replicating virus.

FIG. 8 depicts neutralization of the H7.HK mAbs against H7N9/AH1 in MDCK cells.

FIG. 9 depicts a summary of the genetic composition, epitope, and neutralization function of the isolated H7N9 mAbs.

FIG. 10 depicts protein sequences of the heavy and light chain variable regions of the H7.HK mAbs are aligned to the putative germline V-genes at top, with amino acid substitutions in red, and substitutions shared between the clonally related mAbs H7.HK1 and H7.HK2 in magenta. Spaces are added to maintain alignment; framework regions (FR) and complementarity-determining regions (CDRs) are indicated based on the Kabat numbering and nomenclature. Highlighted in yellow are the mAb residues (paratopes of H7.HK1 and H7.HK2) contacting the H7 antigen. The putative N-linked glycosylation sites on the light chain CDR L1 of H7.HK1 and H7.HK2 and the heavy chain CDR H2 of H7.HK3 are underlined.

FIGS. 11A-11B shows human H7N9 mAbs binding to a soluble, disulfide-stabilized, fully cleaved H7 HA trimer H7 SH13 DS2 6R. FIG. 11A shows ELISA binding curves of the H7.HK mAbs to H7 SH13 DS2 6R HA trimer. FIG. 11B depicts ELISA binding curves of previously reported mAbs to H7 SH13 DS2 6R HA trimer; the H7.HK2mAb was included for comparison. Similar results have been independently reproduced at least once.

FIG. 12 depicts Cryo-EM data processing for H7.HK1 and H7.HK2 in complex with H7 SH13 DS2 6R HA trimer.

FIGS. 13A-13B depict cryo-EM structures of H7.HK1 and H7.HK2 bound to H7 in the head region. FIG. 13A depicts two front views and FIG. 13B depicts a top view of alignment of H7.HK1 and H7.HK2 structures.

FIGS. 14A-14H show cryo-EM details of H7.HK1 and H7.HK2 in complex with H7 SH13 DS2 6R HA trimer. FIG. 14A shows a surface presentation of the H7.HK1 epitope (orange) on H7 HA1, with interacting CDRs shown. FIG. 14B shows H7.HK1 heavy chain forms seven hydrogen bonds and one salt bridge with H7 HA1. FIG. 14C shows that H7.HK1 light chain forms one additional hydrogen bond with H7 HA1, and the interactions are stabilized by hydrophobic residues on the periphery of the light chain interface. FIG. 14D depicts modeling published structures of H7 HA1-binding antibodies (PDB: 6II4, 6II8, 6II9, 5V2A) onto the H7.HK1 bound structure, with an escape mutation R57K (green) reported for mAb 07-5F01. FIG. 14E shows competition ELISA with biotinylated H7.HK2 binding to the H7 HA monomer, in which unlabeled competing mAbs were titrated at increasing concentrations to evaluate the effect on H7.HK2 binding. FIG. 14F shows sequence analysis of N=1,483 H7 HA1s revealed a conserved lateral patch that largely overlaps with the H7.HK1 epitope. FIG. 14G depicts modeling the binding site of human receptor analogue LSTc (red) based on a previous crystal structure (PDB: 4BSE) onto H7 from the H7.HK1 complex, showing that H7.HK1 does not compete with sialic acid on the adjacent protomer (black). FIG. 14H shows alignment of the H7.HK1 complex with a previous crystal structure of H7 (PDB: 4BSE). FIG. 14H shows that the 220-loop (pink) required for sialic acid binding (G218-G228) is disordered in the complex structure and would clash with the H7.HK1 light chain if it were present. Green asterisk symbol denotes the <2 Å clash between the CDR L1 N28 and the predicted location of P221 on HA1.

FIGS. 15A-15G shows cryo-EM details of H7.HK1 and H7.HK2 in complex with H7 SH13 DS2 6R HA trimer. FIG. 15A is a representative micrograph of H7.HK1 and H7.HK2. FIG. 15B is a representative 2D class averages of H7.HK1 and H7.HK2. FIG. 15C shows the gold-standard Fourier Shell Correlation (FSC) resulted in a resolution of 3.62 Å for the overall map of H7.HK1 and 3.69 Å for the overall map of H7.HK2. Non-uniform refinement with C3 symmetry was used for both reconstructions. FIG. 15D depicts the orientations of all particles used in the final refinement are shown as a heatmap. FIG. 15E shows the local resolution of the final overall map is shown contoured at 0.0989 for both structures. Resolution estimation was generated through cryoSPARC using an FSC cutoff of 0.143. FIG. 15F shows representative density is shown for the interface of H7.HK1 heavy chain, light chain, and H7 HA. FIG. 15G depicts representative density for the interface of H7.HK2 heavy chain, light chain, and H7 HA.

FIG. 16 depicts cryo-EM data collection, refinement, and validation statistics for H7 SH13 DS2 6R HA in complex with H7.HK1 and H7.HK2 Fabs.

FIGS. 17A-17C show a comparison of H7.HK1 and H7.HK2 binding to H7. FIG. 17A depicts the difference in epitope of H7.HK1 and H7.HK2, showing that the majority of surface contacts are conserved (shown in orange). FIG. 17B shows hydrogen bonds and salt bridges formed by H7.HK1 and H7.HK2 with H7. FIG. 17C depicts the differences in CDR L2 binding to H7 by H7.HK1 and H7.HK2 as a result of F61S substitution in H7.HK2. S56 forms an additional hydrogen bond with G129 of H7. Additionally, position of Y49 is shifted so that it forms a hydrogen bond with T165 for H7.HK2 instead of Q163 for H7.HK1.

FIGS. 18A-18B depict antigenic drift of H7 HA1 in 2016-2017. FIG. 18A depicts the A/Aichi/2/1968 H3N2 HA1 protein sequence, shown at top to indicate the H3 numbering of HA1. The H7 HA1 sequences from the indicated viral isolates are aligned to the 2013 Hong Kong H7N9 autologous isolate, with identical amino acids shown in dots. “−” depicts gap. Highlighted in yellow are the H7 contact residues (epitope) with both mAbs H7.HK1 and H7.HK2. H7.HK1 specific epitopes are in magenta; H7.HK2 specific epitopes are in cyan. FIG. 18B depicts surface presentation of the H7 HA1 domain highlighting the epitopes (orange) of mAbs H7.HK1 and H7.HK2, with three mutations in red that appeared in the 2016-2017 viral isolates of H7N9. The sticks are interacting CDRs of mAb H7.HK1 heavy and light chains.

FIG. 19 depicts prophylactic effectiveness of human H7N9 mAbs in BALB/c mice i.n. challenged with 10 LD₅₀ of A/Anhui/1/2013 H7N9 virus. FIG. 19 depicts the % survival (less than 20% weight loss) and % body weight plotted over time for an experiment in which mice where i.p. injected 100 μg (equivalent of 5 mg/kg) or 20 μg (equivalent of 1 mg/kg) of the indicated mAbs (as human IgG1 unless otherwise specified) one day before viral challenge. Arrows indicate the time when mAbs were administered. Control groups of a non-H7 placebo mAb and PBS were included. Data for each group were combined from 1-2 experiments and shown as mean−SEM. Asterisk symbols denote statistical significance from two-sided unpaired student's t-test with P values <0.05, and #denote P<0.1.

FIG. 20 depicts therapeutic effectiveness of human H7N9 mAbs in BALB/c mice i.n. challenged with 10 LD₅₀ of A/Anhui/1/2013 H7N9 virus. The % survival and % body weight of mice is plotted over time for an experiment in which mice were i.p. injected 100 μg of the indicated mAbs one day after viral challenge. Arrows indicate the time when mAbs were administered. Control groups of a non-H7 placebo mAb and PBS were included. Data for each group were combined from 1-2 experiments and shown as mean−SEM. Asterisk symbols denote statistical significance from two-sided unpaired student's t-test with P values <0.05, and #denote P<0.1.

FIGS. 21A-21B show that H7.HK1 and two other lateral patch-binding antibodies define a conserved supersite of vulnerability on the HA head. FIG. 21A is a representation of H7.HK1, Fab6649, and 045-09-2B05 bound to their respective HAs indicates diverse angles of approach and heavy/light chain orientations towards the lateral patch supersite. FIG. 21B depicts a comparison of epitopes of H7.HK1, Fab6649, and 045-09-2B05 centering on the lateral patch defines the lateral patch supersite (blue).

FIGS. 22A-22B show that H7.HK1 and two other lateral patch-binding antibodies define a conserved supersite of vulnerability on the HA head. FIG. 22A depicts a subset of epitope surface, centered on the lateral patch, overlaps between H7.HK1, Fab6649, and 045-09-2B05 (magenta). Of shared epitope surface, a subset of epitope residues is conserved (positions 121, 126, 168, and 172 by H3 numbering). FIG. 22B shows that the four lateral patch antibodies contact conserved residues using diverse chemistry. The table displays the type of interaction between each residue and antibody.

FIG. 23 shows the effects of N-linked glycan removal mutations on H7N9 mAbs' binding to the H7 antigen. ELISA binding curves of mAbs H7.HK1 (top), H7.HK2 (middle), and H7.HK3 (bottom) to H7N9 HA1 from A/Shanghai/2/2013 with and without the indicated N-linked glycan removal mutations. “K” stands for kappa chain, and “H” stands for heavy chain.

FIG. 24 shows the effects of N-linked glycan removal mutations on H7N9 mAbs' neutralization against H7N9. Neutralization curves of mAbs H7.HK1 (top), H7.HK2 (middle), and H7.HK3 (bottom) against the indicated 2013 H7N9 pseudo virus in MDCK cells, with and without the indicated N-linked glycan removal mutations. “K” stands for kappa chain, and “H” stands for heavy chain.

### DETAILED DESCRIPTION

H7N9 is an avian influenza A group 2 virus first transmitted to humans mostly via live poultry market exposure in China in the spring of 2013. The virus reemerged in the fall of 2013 and in the winter of later years. Though there is limited evidence for human-to-human transmission, few mutations in the hemagglutinin (HA) gene of the virus might be sufficient to overcome its inefficiency for human transmission (J. Zhou, et al., Biological features of novel avian influenza A (H7N9) virus. Nature 499, 500-503 (2013); Y. Shi, et al., Structures and receptor binding of hemagglutinins from human-infecting H7N9 influenza viruses. Science 342, 243-247 (2013); R. Xu, et al., Preferential recognition of avian-like receptors in human influenza A H7N9 viruses. Science 342, 1230-1235 (2013); and Y. Xu, et al., Avian-to-Human Receptor-Binding Adaptation of Avian H7N9 Influenza Virus Hemagglutinin. Cell Rep 29, 2217-2228 e2215 (2019). H7N9 has been considered one of the most serious pandemic threats. Similar to other influenza virus infections, the only treatment options are antiviral drugs (e.g., oseltamivir, zanamivir, peramivir). The most used antiviral against H7N9 is the neuraminidase inhibitor oseltamivir, but oseltamivir-resistant strains have emerged (Y. Hu, et al., Association between adverse clinical outcome in human disease caused by novel influenza A H7N9 virus and sustained viral shedding and emergence of antiviral resistance. Lancet 381, 2273-2279 (2013); R. Hai, et al., Influenza A (H7N9) virus gains neuraminidase inhibitor resistance without loss of in vivo virulence or transmissibility. Nature communications 4, 2854 (2013); and H. Marjuki, et al., Gubareva, Neuraminidase Mutations Conferring Resistance to Oseltamivir in Influenza A (H7N9) Viruses. Journal of virology 89, 5419-5426 (2015)). Intravenous zanamivir, another neuraminidase inhibitor, though not clinically approved yet, has been used on a compassionate basis in some severe cases because of favorable pharmacokinetics and in vitro susceptibility against oseltamivir-resistant strains (Y. Hu, et al., Association between adverse clinical outcome in human disease caused by novel influenza A H7N9 virus and sustained viral shedding and emergence of antiviral resistance. Lancet 381, 2273-2279 (2013); R. Hai, et al., Influenza A (H7N9) virus gains neuraminidase inhibitor resistance without loss of in vivo virulence or transmissibility. Nature communications 4, 2854 (2013); and H. Marjuki, et al., Neuraminidase Mutations Conferring Resistance to Oseltamivir in Influenza A (H7N9) Viruses. Journal of virology 89, 5419-5426 (2015), each of which are hereby incorporated by reference in its entirety.). However, the effectiveness of intravenous zanamivir against H7N9 has not been validated in large clinical trials. Other antiviral treatment includes the endonuclease inhibitor Xofluza (i.e., baloxavir marboxil) that targets the viral polymerase. Kiso M, Yamayoshi S, Furusawa Y, Imai M, Kawaoka Y. 2019. Treatment of Highly Pathogenic H7N9 Virus-Infected Mice with Baloxavir Marboxil. Viruses 11:1066, which is hereby incorporated by reference in its entirety. Despite the use of neuraminidase and endonuclease inhibitors, the H7N9 case-fatality rate is still more than 30%, and currently there is no licensed vaccine or treatment against H7N9 for humans. An endonuclease inhibitor baloxavir marboxil, targeting the influenza virus polymerase acid, protected mice from lethal H7N9 challenge (Kiso M, Yamayoshi S, Furusawa Y, Imai M, Kawaoka Y. 2019. Treatment of Highly Pathogenic H7N9 Virus-Infected Mice with Baloxavir Marboxil. Viruses 11:1066), but treatment for human H7N9 infection with this inhibitor has not been reported. The concerns for a major outbreak and the lack of effective treatment underscore the unmet need for human monoclonal antibodies (mAbs) with antiviral functions to fight against H7N9. Disclosed herein are H7-reactive human mAbs that bind to neutralization sites on HA and are effective in use for prevention and/or treatment of influenza virus infection such as infection caused by H7N9.

Already endemic, adapted, and evolved in humans for 11 years, H7N9 continues to pose risk and infect human cases exposed to infected poultry in China. While the current risk to public health is low, the pandemic potential of H7N9 is especially concerning if it were to gain the ability of sustained human-to-human transmission. Based on its biological features such as dual affinity for avian and human receptors, high case-fatality rate, resistance to neuraminidase inhibitors, and lack of pre-existing immunity in the human populations, there is an immediate need and interest to develop human mAb prophylaxis and therapeutics against H7N9, to which a specific treatment or licensed vaccine (for humans) is not available.

Previous studies isolated single mAb and showed mixed results regarding protection against H7N9 challenges in mice. (Z. Chen, et al., Human monoclonal antibodies targeting the haemagglutinin glycoprotein can neutralize H7N9 influenza virus. Nature communications 6, 6714 (2015); J. Wang, et al., Characterization of Two Human Monoclonal Antibodies Neutralizing Influenza A H7N9 Viruses. Journal of virology 89, 9115-9118 (2015); N. J. Thornburg, et al., H7N9 influenza virus neutralizing antibodies that possess few somatic mutations. The Journal of clinical investigation 126, 1482-1494 (2016); C. J. Henry Dunand, et al., Both Neutralizing and Non-Neutralizing Human H7N9 Influenza Vaccine-Induced Monoclonal Antibodies Confer Protection. Cell host & microbe 19, 800-813 (2016), each of which is incorporated by reference herein in its entirety.) These studies did not test combinations of two or more mAbs that target different regions of HA.

In the post COVID-19 era, preparedness for future pandemics has become a high priority, as exemplified by the science community closely monitoring a bird flu (avian influenza A H5N1) outbreak in US cows. Kozlov M, Mallapaty S. 2024. Bird flu outbreak in US cows: why scientists are concerned. Nature doi: 10.1038/d41586-024-01036-1, which is hereby incorporated by reference in its entirety.

Disclosed herein are novel human mAbs that, in some embodiments, are used in combination to fight against H7N9. In some embodiments, and without being bound by any theory, the mAbs disclosed herein are capable of potently neutralizing H7N9 through a newly defined site of vulnerability on the head of HA. In some embodiments the neutralizing mAbs disclosed herein are used in combination with one or more non-neutralizing mAb, also directed to the stem of HA, to augment protection against H7N9.

The mAbs disclosed herein, defined by their nucleic acid or amino acid compositions, can be used to treat H7N9-infected patients or to prevent new H7N9 infections in an epidemic region. In some embodiments, the human mAbs described herein have demonstrated anti-viral function against the H7N9 influenza virus and thus could be used alone or in combination to prevent or treat H7N9 human infections.

As disclosed herein, two HA1-directed clonally related human mAbs, H7.HK1 and H7.HK2, neutralized H7N9 with potencies and mouse protection efficacies (both prophylactic and therapeutic) in line with potency and efficacy suitable for clinical use (See Example 19). H7.HK1 and H7.HK2 disclosed herein each potently neutralized the H7N9 2013 pseudo virus with an IC₅₀ of 20 ng/mL. H7.HK1 and H7.HK2 disclosed herein protected mice against a lethal challenge at 1 mg/kg with up to 12% weight loss. Surprisingly, the epitopes of H7.HK1 and H7.HK2 are structurally defined to the β14-centered surface of H7 HA1, partially overlapping with the antigenic site D rather than the commonly targeted receptor binding site (antigenic sites A and B) by prior art H7N9 mAbs (See Example 18). Structural alignments and comparisons demonstrated that antibodies H7.HK1 and H7.HK2 interacted with H7 completely differently from prior art antibodies L4A-14 and H7.167, as an example (see Example 18). The unique β14-targeting epitope by H7.HK1 and H7.HK2 on HA1 would render these mAbs favorable candidates for combination prophylaxis and therapy against H7N9 to augment protection efficacy and increase the genetic barrier for viral escape.

The H7N9 virus has evolved over time and its HA gene has significantly changed in 2016-2017 compared to that of 2013 (with up to 12 amino acid substitutions in HA1). Consequently, most neutralizing mAbs isolated from individuals infected or vaccinated with the 2013 H7 HA lost reactivity to the 2016-2017 isolates, requiring updated H7 immunogens for mAb and vaccine development (K. A. Huang, et al., Structure-function analysis of neutralizing antibodies to H7N9 influenza from naturally infected humans. Nat Microbiol 4, 306-315 (2019), incorporated by reference herein in its entirety). It is demonstrated herein that three mutations that appeared in 2016-2017 are located at the periphery of H7.HK1 and H7.HK2 epitopes and confirmed that the binding profiles of H7.HK1 and H7.HK2 are intact to both 2016 and 2017 HA1s compared to the 2013 HA1 (see Example 17). It was also demonstrated herein that both H7.HK1 and H7.HK2 retained their neutralization titers against the H7N9 2017 pseudo virus with an IC₅₀ of 30 ng/mL. Previous protective mAbs such as HNIgGA6 (Z. Chen, et al., Human monoclonal antibodies targeting the haemagglutinin glycoprotein can neutralize H7N9 influenza virus. Nature communications 6, 6714 (2015), incorporated by reference herein in its entirety), H7.167 (N. J. Thornburg, et al., H7N9 influenza virus neutralizing antibodies that possess few somatic mutations. The Journal of clinical investigation 126, 1482-1494 (2016), incorporated by reference herein in its entirety), and 07-5F01 (C. J. Henry Dunand, et al., Both Neutralizing and Non-Neutralizing Human H7N9 Influenza Vaccine-Induced Monoclonal Antibodies Confer Protection. Cell host & microbe 19, 800-813 (2016), incorporated by reference herein in its entirety) were not evaluated for reactivity to H7N9 2016-2017 isolates. L4A-14 was active against A/Guangdong/TH005/2017 (an avian virus related to A/Guangdong/17SF003/2016) but required 10 mg/kg, compared to 1 mg/kg of H7.HK1 and H7.HK2, for mice protection with up to about 10% weight loss (K. A. Huang, et al., Townsend, Structure-function analysis of neutralizing antibodies to H7N9 influenza from naturally infected humans. Nat Microbiol 4, 306-315 (2019), hereby incorporated by reference in its entirety). Compared to a 2013 H7N9 isolate, the, the neutralization IC₅₀ of 07-5F01 was reduced by more than 100-fold against A/mallard/Netherlands/12/2000 H7N7 compared to a 2013 H7N9 isolate (C. J. Henry Dunand, et al., Both Neutralizing and Non-Neutralizing Human H7N9 Influenza Vaccine-Induced Monoclonal Antibodies Confer Protection. Cell host & microbe 19, 800-813 (2016), incorporated by reference herein in its entirety), and H7.167 did not recognize the H7 from A/Netherlands/219/2003 H7N7 (N. J. Thornburg, et al., H7N9 influenza virus neutralizing antibodies that possess few somatic mutations. The Journal of clinical investigation 126, 1482-1494 (2016), incorporated by reference herein in its entirety), to which all four H7.HK mAbs from the present study bound tightly (see Example 17). Therefore, in some embodiments, the antibodies disclosed herein are advantageous over prior art antibodies against H7N9.

Unexpectedly, a suboptimal dose of H7.HK2 combined with the HA2-directed non-neutralizing mAb H7.HK4 resulted in mouse protection against a lethal challenge. Compared to HA1 (the head region of HA), HA2 (the stalk domain) is genetically more conserved. Hence, HA2-directed mAbs typically display broader recognition of HAs than HA1-directed mAbs. It was surprisingly found that H7.HK4, in addition to H7N9, also recognized the HAs from H10N8 and H15N8, to which both H7.HK1 and H7.HK2 had no reactivity (see Example 17). When converted to mouse IgG2a enabling Fc effector function in mice, H7.HK4 demonstrated measurable prophylactic protection at 5 mg/kg and augmented mouse protection of H7.HK2 (see Example 19), supporting the inclusion of HA2-directed antibodies in a mAb combination regimen against H7N9 in some embodiments. Unexpectedly, the data described herein reveal antibodies directed to a conserved lateral HA1 supersite that confer neutralization, and when combined with a HA2-directed non-neutralizing mAb, augment protection.

Disclosed herein are two isolated clonally related HA1-directed neutralizing mAbs, H7.HK1 and H7.HK2, that demonstrated prophylactic and therapeutic efficacies in a mouse lethal challenge model (see Example 19). Cryo-EM structures revealed a new β14-centered site of vulnerability targeted by H7.HK1 and H7.HK2, those being the first reported antibodies to bind to the H7 lateral patch. Recognition of this conserved epitope facilitates near full binding capacity and neutralization of H7.HK1 and H7.HK2 mAbs to the later 2016-2017 H7N9 isolates (see Example 17-18). In some embodiments, this unique epitope at the lateral patch of HA1 renders H7.HK1 and/or H7.HK2 favorable candidates for combination prophylaxis and therapy against H7N9, which may include multiple HA1-directed neutralizing mAbs targeting different epitopes and benefit from the inclusion of HA2-directed mAbs as well.

H7N9 has evolved over time and its HA gene has significantly changed in 2016-2017 compared to that of 2013. Consequently, most neutralizing mAbs isolated from individuals infected or vaccinated with the 2013 H7 HA lost reactivity to 2016-2017 isolates, requiring updated H7 immunogens for mAb and vaccine development. Huang K A, Rijal P, Jiang H, Wang B, Schimanski L, Dong T, Liu Y M, Chang P, Iqbal M, Wang M C, Chen Z, Song R, Huang C C, Yang J H, Qi J, Lin T Y, Li A, Powell T J, Jan J T, Ma C, Gao G F, Shi Y, Townsend A R. 2019. Structure-function analysis of neutralizing antibodies to H7N9 influenza from naturally infected humans. Nat Microbiol 4:306-315, each of which are hereby incorporated by reference in its entirety. It is demonstrated herein that three mutations appeared in 2016-2017 are located at the periphery of the H7.HK1 and H7.HK2 epitopes. Results described herein confirmed that the binding profiles of H7.HK1 and H7.HK2 are intact to both 2016 and 2017 HA1s as compared to 2013 HA1. It is demonstrated herein that H7.HK1 and H7.HK2 retained their neutralization titers against the H7N9 2017 pseudo virus. The previous RBS-directed mAb HNIgGA6 was shown to lose reactivity to V186G and L226Q mutations (See Chen Z, Wang J, Bao L, Guo L, Zhang W, Xue Y, Zhou H, Xiao Y, Wang J, Wu F, Deng Y, Qin C, Jin Q. 2015. Human monoclonal antibodies targeting the haemagglutinin glycoprotein can neutralize H7N9 influenza virus. Nat Commun 6:6714, incorporated by reference herein in its entirety) that are present in A/Netherlands/219/2003 H7N7 and A/Guangdong/17SF003/2016 H7N9, respectively. Evaluated by both antigen binding and pseudo virus neutralization, H7.HK2 is superior to the two best previous RBS-directed mAbs L4A-14 and H7.167 and matches the one best previous non-RBS mAb 07-5F01 against H7N9.

Described herein, from a 2013 H7N9 convalescent case occurring in Hong Kong, are isolated two clonally related HA1-directed neutralizing mAbs H7.HK1 and H7.HK2 that demonstrated prophylactic and therapeutic efficacies in a mouse lethal challenge model. Cryo-EM structures revealed a β14-centered site of vulnerability targeted by H7.HK1 and H7.HK2, those being the first reported antibodies to bind to the H7 lateral patch. Recognition of this conserved epitope facilitates near full binding and neutralization capacity of H7.HK1 and H7.HK2 to the later 2016-2017 H7N9 isolates (see Example 20). This unique epitope at the lateral patch of HA1 renders H7.HK1 and H7.HK2 favorable candidates for combination prophylaxis and therapy against H7N9, which may include multiple HA1-directed neutralizing mAbs targeting different epitopes and benefit from the inclusion of HA2-directed mAbs as well.

Unexpectedly, in some embodiments, antibodies containing N-linked glycan removal mutations can effectively bind to H7N9 HA1 and neutralizeH7N9 viruses (see Example 21). Also unexpectedly, in some embodiments, antibodies containing N-linked glycan removal mutations show enhanced binding to H7N9 HA1 and improved neutralization function against H7N9 at lower concentrations compared to their wild-type counterparts (i.e., antibodies without N-linked glycan removal mutations) (see Example 21).

### Antibodies

There are five classes of human antibodies (i.e., IgA, IgD, IgE, IgG, and IgM) and each have various isotypes (e.g., IgG1, IgG2, IgG3, IgG4, IgA1, and IgA2). In some embodiments, the antibodies disclosed herein belong to the IgG class. IgG can be further divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety.

The IgG immunoglobulin molecule consists of four polypeptide chains, two identical light (L) chains and two identical heavy (H) chains. The four chains are joined by disulfide bonds in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, C T, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. See Kabat et al., Sequences of Immunological Interest, Fifth Edition, National Institute of Health, Bethesda, MD (1991), incorporated by reference in its entirety herein. The constant domains are not involved directly in the binding of antibody to an antigen, but exhibit various effector functions, such as participation of the antibody in antibody-dependent cellular toxicity.

By way of example, CDRs may be defined using the nomenclature described by Kabat et al. (1991, NIH Publication 91-3242, National Technical Information Service, Springfield, Va.), incorporated by reference in its entirety herein. Specifically, residues 31-35 (CDR-H1), 50-65 (CDR-H2), and 95-102 (CDR-H3) in the heavy chain variable region and residues 24-34 (CDR-L1), 50-56 (CDR-L2), and 89-97 (CDR-L3) in the light chain variable region.

There are differences in the structures of the four IgG subclasses: IgG1, IgG2, IgG3, and IgG4. For example, there is variation in the hinge region and the N-terminal CH2 domains, as well as in the CH2 and CH3 domains that form the Fc tail. The four subclasses differ with respect to the number of inter-heavy chain disulfide bonds in the hinge region. Variations in the hinge region are responsible for differences in effector functions. For example, the hinge of IgG2 is shorter and more rigid than the other subclasses including IgG1. E.g., see Gestur Vidarsson, et al. (2014) hereby incorporated by reference in its entirety. Without intending to be bound by any theory, the Fc region of IgG2 plays an important role in the protective function of antibodies disclosed herein.

However, the antibodies disclosed herein are not limited to full-length antibodies. The antibodies of the various embodiments disclosed herein can include one or more of synthetic antibodies, monoclonal antibodies, oligoclonal or polyclonal antibodies, multiclonal antibodies, recombinantly produced antibodies, intrabodies, multispecific antibodies, bispecific antibodies, monovalent antibodies, multivalent antibodies, human antibodies, humanized antibodies, chimeric antibodies, CDR-grafted antibodies, primatized antibodies, Fab fragments, F(ab′) fragments, F(ab′) 2 fragments, Fv fragments, single-chain FvFcs (scFvFc), single-chain Fvs (scFv), Dabs, nanobodies, anti-idiotypic (anti-Id) antibodies, and any other immunologically-reactive/antigen-binding fragments thereof.

In some embodiments, the antibodies disclosed herein contain various modifications, substitutions, additions or deletions to the variable or binding regions of anti-H7N9 antibodies disclosed herein. In some embodiments, the antibodies disclosed herein may contain substitutions or modifications of the constant region (i.e., the Fc region). The antibodies disclosed herein may contain one or more additional amino acid residue substitutions, mutations and/or modifications, which result in a compound with preferred characteristics including, but not limited to: altered pharmacokinetics, increased serum half-life, increase binding affinity, reduced immunogenicity, increased production, altered Fc ligand binding, enhanced or reduced ADCC or CDC activity, altered glycosylation and/or disulfide bonds and modified binding specificity.

Amino Acid Sequences of Human Monoclonal Antibodies (mAb) to H7N9 HA

Underlined amino acids represent the respective complementarity determining regions (CDRs) and italicized and bolded amino acids represent the respective framework regions (FR). Glycosylation sites (which are part of the CDRs) are shown in double underline.

In some embodiments, the amino acid sequence of the variable heavy chain domain of an anti-H7N9 HA antibody comprises SEQ ID NO: 1.

(SEQ ID NO: 1)
QVQLQESGPGLVKPSETLSLTCSVSGGSINSYYWTWIRQPPGKGLEWVG
YIYHSGSTSYNPSLKSRITISVAPSKNHFSLELTSMTAADTAVYYCARL
GGHGDYGSDYWGQGTLVTVSS.

The corresponding variable light chain domain of the anti-H7N9 HA antibody comprises SEQ ID NO: 2.

(SEQ ID NO: 2)
DIVMTQSPVSLPVTPGEPASISCNSSQSLLHSNGYAHLDWYLQKPGQSP
KLMIYLGLNRAFGVPDRFSGSGSGTDFTLKISRVEAEDVGVYYCMQALQ
TPFTFGPGTRVDIK.

The antibody comprising SEQ ID NO: 1 and SEQ ID NO: 2 is represented by antibody “H7.HK1” in embodiments described and depicted in this disclosure.

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 1 or SEQ ID NO: 2. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 1 or SEQ ID NO: 2.

In some embodiments, the amino acid sequence of the variable heavy chain domain of an anti-H7N9 HA antibody comprises SEQ ID NO: 3.

(SEQ ID NO: 3)
QVQLQGSGPGLLRPSETLSLTCSVSGVSINSYYWSWVRQPPGKALEWIG
YIYYSGNTNYNPSLESRVTISVDRSKNQFSLKMTSVTAADTARYFCARQ
GIFGDYGSDYWGPGTLVTVSS.

The corresponding variable light chain domain of the anti-H7N9 HA antibody comprises SEQ ID NO: 4.

(SEQ ID NO: 4)
DIVMTQSPLSLPVTPGEPASISCRSNQSLQHSNGYVHLDWYRQKPGQSP
HLLIYLGFNRASGVPDRFSGGGSGTDFTLKISRVEAEDVGVYYCMQGLQ
TPFTFGPGTTVDLK.

The antibody comprising SEQ ID NO: 3 and SEQ ID NO: 4 is represented by antibody “H7.HK2” in embodiments described and depicted in this disclosure.

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 3 or SEQ ID NO: 4. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3 or SEQ ID NO: 4.

In some embodiments, the amino acid sequence of the variable heavy chain domain of an anti-H7N9 HA antibody comprises SEQ ID NO: 5.

(SEQ ID NO: 5)
QVQLVQSGSELKRPGASVKVSCRASGYTFTSYTINWVRQAPGQGLEWMG
WINTSTGDPTYAQGFTGRFVFSLDTSVSTAYLEISRLKAEDTAVYYCAR
AFGLTVVRGGIVGVWGQGTTVTVSS.

The corresponding variable light chain domain of the anti-H7N9 HA antibody comprises SEQ ID NO: 6.

(SEQ ID NO: 6)
DIQMTQSPSTLSASVGDRVTITCRASQSISSWLAWYQQKPGKAPKLLIY
KASSLESGVPSRFSGSGSGTEFTLTISSLQPDDFATYYCQQYNSYSQTF
GQGTKVEIK.

The antibody comprising SEQ ID NO: 5 and SEQ ID NO: 6 is represented by antibody “H7.HK3” in embodiments described and depicted in this disclosure.

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 5 or SEQ ID NO: 6. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 5 or SEQ ID NO: 6.

In some embodiments, the amino acid sequence of the variable heavy chain domain of an anti-H7N9 HA antibody comprises SEQ ID NO: 7.

(SEQ ID NO: 7)
QVQLQESGPGLVKPSETLSLTCTVSGGSVRSASYAWSWIRQPPGKGLEW
IGDIYYSGTTNYNPSLKSRVTLSVDTAKNRFSLRLRSVTAADTAVYHCA
RERYYYGSSGDFDYWGQGTLVTVSS.

The corresponding variable light chain domain of the anti-H7N9 HA antibody comprises SEQ ID NO: 8.

(SEQ ID NO: 8)
DIQMTQSPSSLSASVGDRVTITCRASQGIRNYLAWFQQKPGQAPKSLIF
AASSLHTGVPSRFSGSGSGTDFTLTISSLQPEDFATYYCQHYNSYPPTF
GQGTKLEIK.

The antibody comprising SEQ ID NO: 7 and SEQ ID NO: 8 is represented by antibody “H7.HK4” in embodiments described and depicted in this disclosure.

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 7 or SEQ ID NO: 8. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 7 or SEQ ID NO: 8.

Nucleic Acid Sequences of Human Monoclonal Antibodies (mAb) to H7N9 HA

Underlined nucleic acids represent the respective complementarity determining regions (CDRs) and italicized and bolded nucleic acids represent the respective framework regions (FR). Glycosylation sites (which are part of the CDRs) are shown in double underline.

In some embodiments, the nucleic acid sequence of the variable heavy chain domain of an anti-H7N9 HA antibody comprises SEQ ID NO: 9.

(SEQ ID NO: 9)
CAGGTGCAGCTGCAGGAGTCGGGCCCCGGACTGGTGAAGCCTTCGGAGA
CCCTGTCCCTCACCTGCAGTGTCTCTGGTGGCTCCATCAATAGTTACTA
CTGGACCTGGATTCGGCAGCCCCCGGGGAAGGGACTGGAGTGGGTTGGC
TATATTTATCACAGTGGAAGCACCAGCTACAACCCCTCCCTCAAGAGTC
GAATCACCATTTCCGTAGCCCCGTCCAAGAACCACTTCTCCCTGGAGCT
GACCTCTATGACCGCTGCAGACACGGCCGTCTATTACTGTGCGAGACTG
GGGGGCCACGGTGACTACGGTTCCGACTACTGGGGCCAGGGAACCCTGG
TCACCGTCTCCTCA.

The corresponding variable light chain domain of the anti-H7N9 HA antibody comprises SEQ ID NO: 10.

(SEQ ID NO: 10)
GATATTGTGATGACTCAGTCTCCAGTCTCCCTGCCCGTCACTCCTGGAG
AGCCGGCCTCCATCTCCTGCAACTCTAGTCAGAGCCTCCTGCATAGCAA
TGGATACGCCCATTTGGATTGGTACCTGCAGAAGCCAGGGCAGTCTCCA
AAGCTCATGATCTACCTGGGTCTTAATCGGGCCTTCGGGGTCCCTGACA
GGTTCAGTGGCAGTGGATCAGGCACAGATTTTACACTGAAAATCAGCAG
AGTGGAGGCTGAGGATGTTGGCGTTTATTACTGCATGCAAGCTCTACAA
ACTCCATTCACTTTCGGCCCTGGGACCAGAGTGGATATCAAA.

The antibody comprising SEQ ID NO: 9 and SEQ ID NO: 10 is represented by antibody “H7.HK1” in embodiments described and depicted in this disclosure.

In some embodiments, the variable heavy or light chain domains of the anti-H7N9 HA antibody comprise a nucleic acid sequence 80, 81, 82, 83, 84, 85, 86, 87, 88, 89, 90, 91, 92, 93, 94, 95, 96, 97, 98 or 99% identical to SEQ ID NO: 9 or SEQ ID NO: 10. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 9 or SEQ ID NO: 10.

In some embodiments, the nucleic acid sequence of the variable heavy chain domain of an anti-H7N9 HA antibody comprises SEQ ID NO: 11.

(SEQ ID NO: 11)
CAGGTGCAGCTGCAAGGGTCGGGCCCAGGACTGCTGAGGCCTTCGGAGA
CCCTGTCCCTCACCTGCTCTGTCTCCGGTGTCTCCATCAATAGTTACTA
TTGGAGTTGGGTCCGGCAGCCCCCGGGGAAGGCACTTGAGTGGATTGGC
TATATCTATTATAGTGGCAACACCAACTACAATCCCTCCCTCGAGAGTC
GAGTCACCATATCAGTGGACAGGTCCAAGAACCAGTTCTCCCTGAAGAT
GACCTCTGTGACCGCTGCGGATACGGCCAGATATTTCTGTGCGAGACAG
GGGATCTTCGGTGACTACGGCTCCGACTACTGGGGCCCGGGAACCCTGG
TCACCGTCTCCTCA .

The corresponding variable light chain domain of the anti-H7N9 HA antibody comprises SEQ ID NO: 12.

(SEQ ID NO: 12)
GATATTGTGATGACTCAGTCTCCACTCTCCCTGCCCGTCACCCCTGGAG
AGCCGGCCTCCATCTCCTGTAGGTCTAATCAGAGCCTCCAGCATAGTAA
TGGATACGTCCATTTGGATTGGTACAGGCAGAAGCCAGGGCAGTCTCCA
CACCTCCTGATCTACTTGGGCTTTAATCGGGCCTCCGGGGTCCCTGACA
GGTTCAGTGGCGGTGGATCAGGCACAGATTTTACACTGAAAATCAGCAG
AGTGGAGGCTGAGGATGTTGGAGTTTATTATTGCATGCAAGGTCTACAA
ACTCCATTCACTTTCGGCCCTGGGACCACAGTGGATCTCAAA.

The antibody comprising SEQ ID NO: 11 and SEQ ID NO: 12 is represented by antibody “H7.HK2” in embodiments described and depicted in this disclosure.

In some embodiments, the variable heavy or light chain domains of the anti-H7N9 HA antibody comprise a nucleic acid sequence 80, 81, 82, 83, 84, 85, 86, 87, 88, 89, 90, 91, 92, 93, 94, 95, 96, 97, 98 or 99% identical to SEQ ID NO: 11 or SEQ ID NO: 12. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 11 or SEQ ID NO: 12.

In some embodiments, the nucleic acid sequence of the variable heavy chain domain of an anti-H7N9 HA antibody comprises SEQ ID NO: 13.

(SEQ ID NO: 13)
CAAGTGCAGCTGGTGCAATCTGGGTCTGAGTTGAAGAGGCCTGGGGCCT
CAGTGAAGGTTTCCTGTAGGGCTTCTGGGTACACCTTCACTAGTTATAC
TATCAACTGGGTGCGACAGGCCCCTGGACAAGGGCTTGAGTGGATGGGA
TGGATCAACACCAGCACTGGGGACCCAACATATGCCCAGGGCTTCACAG
GACGGTTTGTCTTCTCCTTGGACACCTCTGTCAGCACGGCATATCTGGA
GATCAGCAGGCTAAAGGCTGAAGACACTGCCGTGTATTACTGTGCGAGA
GCCTTCGGCCTTACTGTGGTTCGGGGAGGTATTGTCGGCGTCTGGGGCC
AAGGGACCACGGTCACCGTCTCCTCA.

The corresponding variable light chain domain of the anti-H7N9 HA antibody comprises SEQ ID NO: 14.

(SEQ ID NO: 14)
GACATCCAGATGACCCAGTCTCCTTCCACCCTGTCTGCATCTGTAGGAG
ACAGAGTCACCATCACTTGCCGGGCCAGTCAGAGTATTAGTAGCTGGTT
GGCCTGGTATCAGCAGAAACCAGGGAAAGCCCCTAAGCTCCTGATCTAT
AAGGCGTCTAGTTTAGAAAGTGGGGTCCCATCAAGGTTCAGCGGCAGTG
GATCTGGGACAGAATTCACTCTCACCATCAGCAGCCTGCAGCCTGATGA
TTTTGCAACTTATTACTGCCAACAGTATAATAGTTATTCTCAGACGTTC
GGCCAAGGGACCAAGGTGGAAATCAAA.

The antibody comprising SEQ ID NO: 13 and SEQ ID NO: 14 is represented by antibody “H7.HK3” in embodiments described and depicted in this disclosure.

In some embodiments, the variable heavy or light chain domains of the anti-H7N9 HA antibody comprise a nucleic acid sequence 80, 81, 82, 83, 84, 85, 86, 87, 88, 89, 90, 91, 92, 93, 94, 95, 96, 97, 98 or 99% identical to SEQ ID NO: 13 or SEQ ID NO: 14. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 13 or SEQ ID NO: 14.

In some embodiments, the nucleic acid sequence of the variable heavy chain domain of an anti-H7N9 HA antibody comprises SEQ ID NO: 15.

(SEQ ID NO: 15)
CAGGTGCAGCTGCAGGAGTCGGGCCCAGGACTGGTGAAGCCTTCGGAGA
CCCTGTCCCTCACTTGCACTGTGTCTGGTGGCTCCGTCAGAAGTGCTAG
TTACGCCTGGAGCTGGATCCGGCAGCCCCCTGGGAAAGGACTGGAGTGG
ATTGGCGATATCTATTACAGTGGGACCACCAATTACAACCCGTCCCTCA
AGAGTCGAGTCACCCTATCGGTCGACACGGCCAAGAACCGGTTCTCCCT
GAGGCTGAGGTCTGTGACCGCTGCGGACACGGCCGTGTATCACTGTGCG
AGAGAGAGGTATTACTATGGTAGTAGTGGTGACTTTGACTACTGGGGCC
AGGGAACCCTGGTCACCGTCTCCTCA.

The corresponding variable light chain domain of the anti-H7N9 HA antibody comprises SEQ ID NO: 16.

(SEQ ID NO: 16)
GACATCCAGATGACCCAGTCTCCATCCTCACTGTCTGCATCTGTAGGCG
ACAGAGTCACCATCACTTGTCGGGCAAGTCAGGGCATTAGAAATTATTT
AGCCTGGTTTCAGCAGAAACCAGGGCAAGCCCCTAAGTCCCTGATCTTT
GCTGCATCCAGTTTGCACACTGGGGTCCCATCGAGGTTCAGCGGCAGTG
GATCTGGGACAGATTTCACTCTCACCATCAGCAGCCTGCAGCCTGAGGA
TTTTGCAACATATTACTGCCAACACTATAATAGTTACCCTCCCACTTTT
GGCCAGGGGACCAAGCTGGAGATCAAA.

The antibody comprising SEQ ID NO: 15 and SEQ ID NO: 16 is represented by antibody “H7.HK4” in embodiments described and depicted in this disclosure.

In some embodiments, the variable heavy or light chain domains of the anti-H7N9 HA antibody comprise a nucleic acid sequence 80, 81, 82, 83, 84, 85, 86, 87, 88, 89, 90, 91, 92, 93, 94, 95, 96, 97, 98 or 99% identical to SEQ ID NO: 15 or SEQ ID NO: 16. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain of the anti-H7N9 antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 15 or SEQ ID NO: 16.

### Optimized Anti-H7N9 Antibodies

In some embodiments, the amino acid sequence of the anti-H7N9 antibody comprises amino acid substitutions that optimize the function of the anti-H7N9 antibody. In some embodiments, the amino acid sequence of the anti-H7N9 antibody comprises amino acid substitutions that increase the affinity of the anti-H7N9 antibody to an HA protein (e.g., HA protein of H7N9). In some embodiments, the variable heavy chain domain or the variable light chain domain of an anti-H7N9 antibody comprises amino acid substitutions relative to SEQ ID NOs: 1-16.

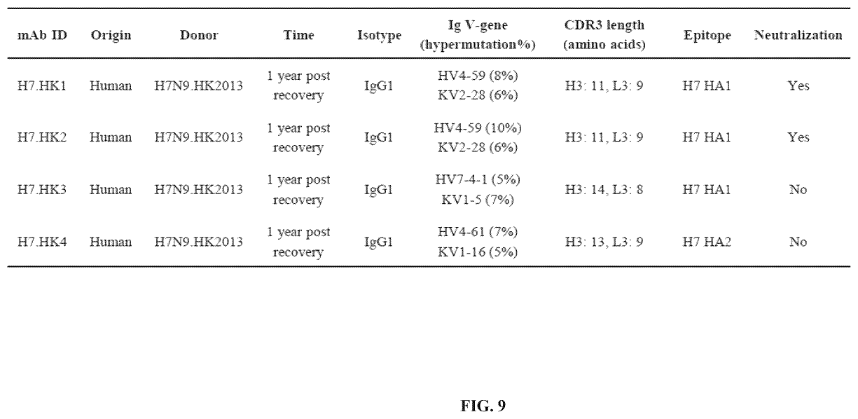

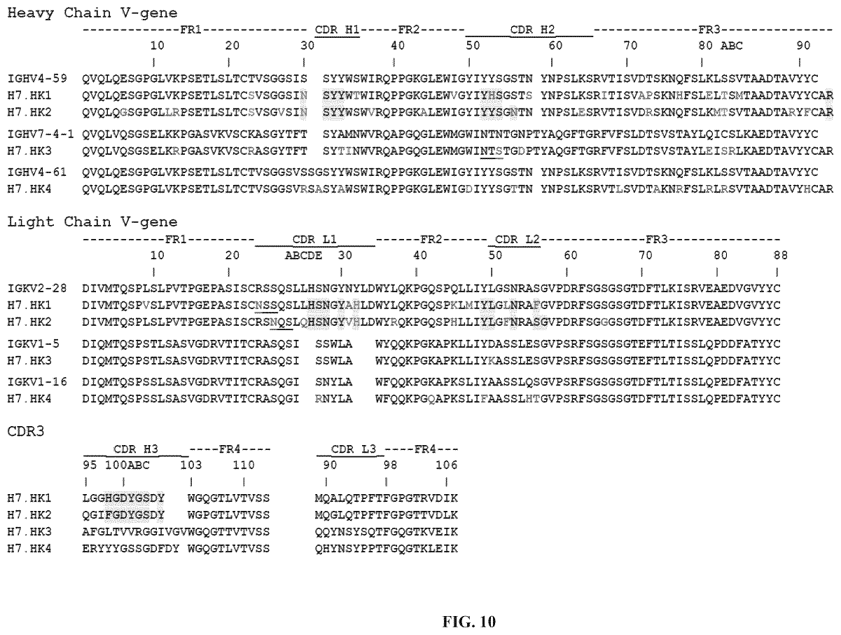

In some embodiments, the anti-H7N9 antibody comprises one or more amino acid substitutions at one or more N-linked glycosylation sites (e.g., see glycosylation sites double underlined in the sequences disclosed herein; see also FIG. 10). In some embodiments, the one or more amino acid substitutions at the one or more N-linked glycosylation sites improves the production of the anti-H7N9 antibody. In some embodiments, the one or more amino acid substitutions at the one or more N-linked glycosylation sites is in the light chain CDR1 of the antibody. In some embodiments, the one or more amino acid substitutions at the one or more N-linked glycosylation sites comprises substituting S in the “NSS” amino acid portion of SEQ ID NO: 2 to A (i.e., NSS is substituted with NSA). Thus, in some embodiments, the variable light chain domain of the anti-H7N9 HA antibody comprises SEQ ID NO: 17.

(SEQ ID NO: 17)
DIVMTQSPVSLPVTPGEPASISCNSAQSLLHSNGYAHLDWYLQKPGQSP
KLMIYLGLNRAFGVPDRFSGSGSGTDFTLKISRVEAEDVGVYYCMQALQ
TPFTFGPGTRVDIK.

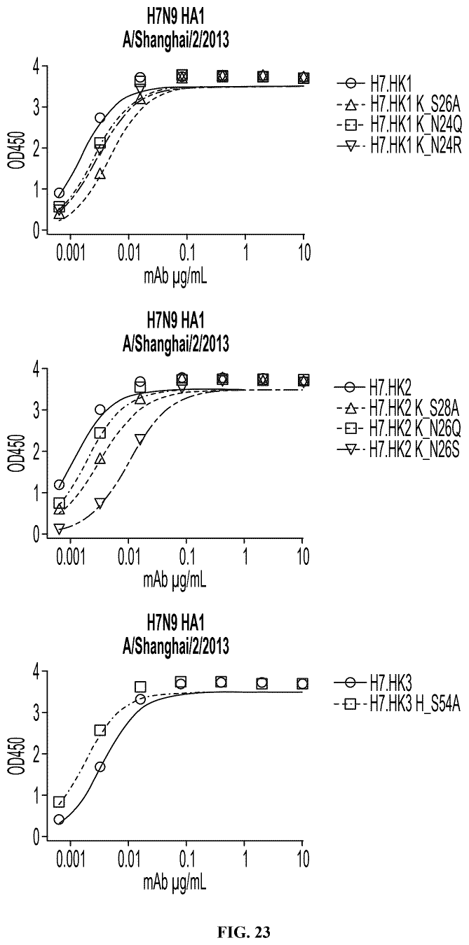

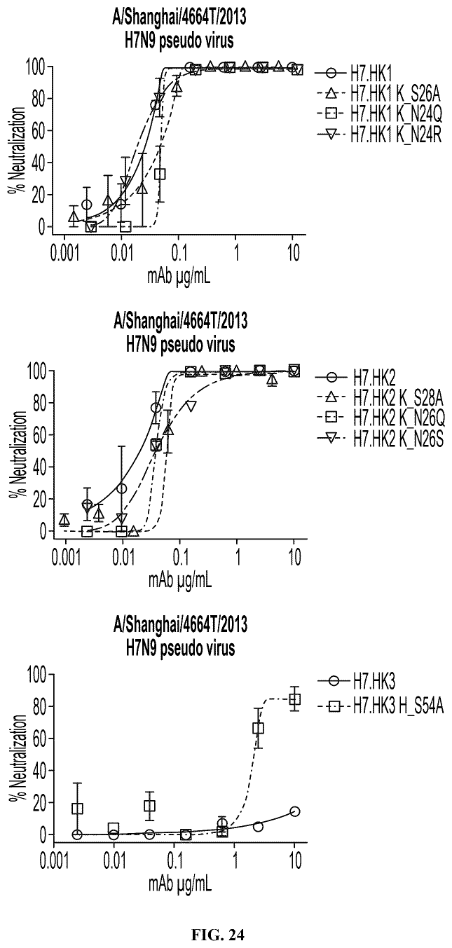

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 1 (heavy chain) and SEQ ID NO: 17 (light chain) is represented by antibody “H7.HK1 K_S26A” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 17. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 17.

In some embodiments, the one or more amino acid substitutions at the one or more N-linked glycosylation sites comprises substituting S in the “NQS” amino acid portion of SEQ ID NO: 4 to A (i.e., NQS is substituted with NQA). Thus, in some embodiments, the variable light chain domain of the anti-H7N9 HA antibody comprises SEQ ID NO: 18.

(SEQ ID NO: 18)
DIVMTQSPLSLPVTPGEPASISCRSNQALQHSNGYVHLDWYRQKPGQSP
HLLIYLGFNRASGVPDRFSGGGSGTDFTLKISRVEAEDVGVYYCMQGLQ
TPFTFGPGTTVDLK.

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 3 (heavy chain) and SEQ ID NO: 18 (light chain) is represented by antibody “H7.HK2 K_S28A” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 18. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 18.

In some embodiments, the one or more amino acid substitutions at the one or more N-linked glycosylation sites comprises substituting N in the “NSS” amino acid portion of SEQ ID NO: 2 to Q (i.e., NSS is substituted with QSS). Thus, in some embodiments, the variable light chain domain of the anti-H7N9 HA antibody comprises SEQ ID NO: 19.

(SEQ ID NO: 19)
DIVMTQSPVSLPVTPGEPASISCQSSQSLLHSNGYAHLDWYLQKPGQSP
KLMIYLGLNRAFGVPDRFSGSGSGTDFTLKISRVEAEDVGVYYMQALQT
PFTFGPGTRVDIK.

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 1 (heavy chain) and SEQ ID NO: 19 (light chain) is represented by antibody “H7.HK1 K_N24Q” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 19. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 19.

In some embodiments, the one or more amino acid substitutions at the one or more N-linked glycosylation sites comprises substituting N in the “NQS” amino acid portion of SEQ ID NO: 4 to Q (i.e., NQS is substituted with QQS). Thus, in some embodiments, the variable light chain domain of the anti-H7N9 HA antibody comprises SEQ ID NO: 20.

(SEQ ID NO: 20)
DIVMTQSPLSLPVTPGEPASISCRSQQSLQHSNGYVHLDWYRQKPGQSP
HLLIYLGFNRASGVPDRFSGGGSGTDFTLKISRVEAEDVGVYYCMQGLQ
TPFTFGPGTTVDLK.

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 3 (heavy chain) and SEQ ID NO: 20 (light chain) is represented by antibody “H7.HK2 K_N26Q” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 20. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 20.

In some embodiments, the one or more amino acid substitutions at the one or more N-linked glycosylation sites comprises substituting N in the “NSS” amino acid portion of SEQ ID NO: 2 to R (i.e., NSS is substituted with RSS) to revert to a germline sequence. Thus, in some embodiments, the variable light chain domain of the anti-H7N9 HA antibody comprises SEQ ID NO: 21.

(SEQ ID NO: 21)
DIVMTQSPVSLPVTPGEPASISCRSSQSLLHSNGYAHLDWYLQKPGQSP
KLMIYLGLNRAFGVPDRFSGSGSGTDFTLKISRVEAEDVGVYYCMQALQ
TPFTFGPGTRVDIK.

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 1 (heavy chain) SEQ ID NO: 21 (light chain) is represented by antibody “H7.HK1 K_N24R” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 21. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 21.

In some embodiments, the one or more amino acid substitutions at the one or more N-linked glycosylation sites comprises substituting N in the “NQS” amino acid portion of SEQ ID NO: 4 to S (i.e., NQS is substituted with SQS) to revert to a germline sequence. Thus, in some embodiments, the variable light chain domain of the anti-H7N9 HA antibody comprises SEQ ID NO: 22.

(SEQ ID NO: 22)
DIVMTQSPLSLPVTPGEPASISCRSSQSLQHSNGYVHLDWYRQKPGQSP
HLLIYLGFNRASGVPDRFSGGGSGTDFTLKISRVEAEDVGVYYCMQGLQ
TPFTFGPGTTVDLK.

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 3 (heavy chain) and SEQ ID NO: 22 (light chain) is represented by antibody “H7.HK2 K_N26S” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 22. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 22.

In some embodiments, the one or more amino acid substitutions at the one or more N-linked glycosylation sites comprises substituting Serine in the “NTS” amino acid portion of SEQ ID NO: 5 to Alanine (i.e., NTS is substituted with NTA). Thus, in some embodiments, the variable heavy chain domain of the anti-H7N9 HA antibody comprises SEQ ID NO: 23.

(SEQ ID NO: 23)
QVQLVQSGSELKRPGASVKVSCRASGYTFTSYTINWVRQAPGQGLEWMG
WINTATGDPTYAQGFTGRFVFSLDTSVSTAYLEISRLKAEDTAVYYCAR
AFGLTVVRGGIVGVWGQGTTVTVSS.

Underlined amino acids represent the respective complementarity determining regions (CDRs) and italicized and bolded nucleic acids represent the respective framework regions (FR). Glycosylation sites (which are part of the CDRs) are shown in double underline. The antibody comprising SEQ ID NO: 23 (heavy chain) and SEQ ID NO: 6 (light chain) is represented by antibody “H7.HK3 H_S54A” in embodiments described and depicted in this disclosure (e.g., see Example 21 and FIGS. 23 and 24).

In some embodiments, the variable heavy or light chain domains of the anti-H7N9 HA antibody comprise an amino acid sequence 80, 81, 82, 83, 84, 85, 86, 87, 88, 89, 90, 91, 92, 93, 94, 95, 96, 97, 98 or 99% identical to SEQ ID NO: 23. In some embodiments, the CDRs of the variable heavy chain domain and the CDRs of the variable light chain domain of the anti-H7N9 antibody comprise the CDR sequences underlined above, while the framework regions (FRs) of the variable heavy chain domain and the FRs of the variable light chain domain the anti-H7N9 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 23.

In some embodiments, the variable heavy chain domain of any one of the anti-H7N9 antibodies disclosed herein can be coupled with a variable heavy chain domain of any one of the other anti-H7N9 antibodies disclosed herein. For example, in some embodiments, the anti-H7N9 antibody comprises the variable heavy chain domain of H7.HK1 and the variable light chain domain of H7.HK2. In some embodiments, the anti-H7N9 antibody comprises the variable heavy chain domain of H7.HK2 and the variable light chain domain of H7.HK1. In some embodiments, the variable heavy chain domain of the anti-H7N9 antibody comprises SEQ ID NO: 1, and the variable light chain domain of the anti-H7N9 antibody comprises SEQ ID NO: 4. In some embodiments, the variable heavy chain domain of the anti-H7N9 antibody comprises SEQ ID NO: 3, and the variable light chain domain of the anti-H7N9 antibody comprises SEQ ID NO: 2.

### Structure of Anti-H7N9 Antibodies

In some embodiments, the molecular three-dimensional structure of an anti-H7N9 antibody can be predicted based on X-ray crystallography, and/or cryo-EM, and/or using structure prediction algorithms (e.g., machine learning algorithms) known in the art, such as AlphaFold or RaptorX. In some embodiments, the structure prediction algorithm is a computational method that is used to predict three-dimensional (3D) antibody structures based on a given nucleic acid or amino acid sequence. In some embodiments, the structure prediction algorithm predicts the 3D coordinates of all heavy atoms for a given antibody using a nucleic acid or amino acid sequence and/or aligned sequences of homologues as inputs. In some embodiments, the structure of the anti-H7N9 antibody is predicted using a combination of methods, e.g., using a combination of AlphaFold (or any other structure prediction algorithm known in the art) and X-ray crystallography or cryo-EM. In some embodiments, the structure prediction is improved by combining the use of AlphaFold (or any other structure prediction algorithm known in the art) and X-ray crystallography or cryo-EM. In some embodiments, the structure of the anti-H7N9 antibody is predicted by using a computational structure prediction algorithm (e.g., AlphaFold or RaptorX) and the structure prediction of the anti-H7N9 antibody is then refined by using X-ray crystallography or cryo-EM. In some embodiments, the anti-H7N9 antibody comprises a three-dimensional structure that is similar to the three-dimensional structure of the anti-H7N9 antibody that comprises a variable heavy domain comprising SEQ ID NO: 1, 3, 5, or 7 and a variable light chain domain comprising SEQ ID NO: 2, 4, 6, or 8.

In some embodiments, the structure prediction algorithm can be used to model the structure of a first anti-H7N9 antibody (e.g., a reference anti-H7N9 antibody; e.g., an anti-H7N9 antibody comprising any of SEQ ID NO. 1-8) and compare a predicted structure of second antibody against the predicted structure of the first antibody such that the second antibody can be categorized in the same class as the first antibody based on its structural similarity to the first antibody. In some embodiments, a metric of structural similarity between two antibodies can be obtained based on the output of a structure prediction algorithm known in the art. In some embodiments, the metric of structural similarity between two antibodies is based on a similarity distance.

In some embodiments, the structure of the anti-H7N9 antibody allows the anti-H7N9 antibody to bind to the β14-centered surface of H7 HA1, partially overlapping with the antigenic site D of the globular head of HA of H7N9. In some embodiments, disclosed herein is a new class of anti-H7N9 antibodies which comprise structural similarity to one another wherein the new class of anti-H7N9 antibodies are capable of binding to the β14-centered surface of H7 HA1, partially overlapping with the antigenic site D of the globular head of HA of H7N9. In some embodiments, the anti-H7N9 antibody is a means for binding a hemagglutinin (HA) protein of an H7N9 virus.

In some embodiments, the anti-H7N9 antibody cross-competes for binding to the HA of H7N9 with one or more of the anti-H7N9 antibodies disclosed herein (e.g., an anti-H7N9 antibody comprising at least one of SEQ ID NO: 1, 2, 3, 4, 5, 6, 7, 8, 17, 18, 19, 20, 21, 22, or 23). In some embodiments, the anti-H7N9 antibody that cross-competes for binding with one or more of the anti-H7N9 antibodies disclosed herein (e.g., an anti-H7N9 antibody comprising at least one of SEQ ID NO: 1, 2, 3, 4, 5, 6, 7, 8, 17, 18, 19, 20, 21, 22, or 23) is structurally similar to one or more of the anti-H7N9 antibodies disclosed herein (e.g., an anti-H7N9 antibody comprising at least one of SEQ ID NO: 1, 2, 3, 4, 5, 6, 7, 8, 17, 18, 19, 20, 21, 22, or 23). For example, in some embodiments, the anti-H7N9 antibody competes with H7.HK1, H7.HK2, H7.HK3, and/or H7.HK4. In some embodiments, the anti-H7N9 antibody competes with H7.HK1, H7.HK2, H7.HK3, and/or H7.HK4 and is structurally similar to H7.HK1, H7.HK2, H7.HK3, and/or H7.HK4.

### Non-Limiting Embodiments of the Subject Matter

In one aspect, described herein is a vector comprising at least one polynucleotide sequence encoding an antibody or fragment thereof, wherein the antibody or fragment thereof comprises a variable heavy chain domain and a variable light chain domain, wherein the variable heavy chain domain of the antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23 and the variable light chain domain of the antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the variable heavy chain domain of the antibody or fragment thereof comprises the amino acid sequence of SEQ ID NO: 1, and the variable light chain domain of the antibody or fragment thereof comprises the amino acid sequence of SEQ ID NO: 17. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 19. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 21. In some embodiments, the variable heavy chain domain of the antibody or fragment thereof comprises the amino acid sequence of SEQ ID NO: 3, and the variable light chain domain of the antibody or fragment thereof comprises the amino acid sequence of SEQ ID NO: 18. In some embodiments, the variable heavy chain domain of the antibody or fragment thereof is the amino acid sequence of SEQ ID NO: 3, and the variable light chain domain of the antibody or fragment thereof is the amino acid sequence of SEQ ID NO: 20. In some embodiments, the variable heavy chain domain of the antibody or fragment thereof comprises the amino acid sequence of SEQ ID NO: 3, and the variable light chain domain of the antibody or fragment thereof comprises the amino acid sequence of SEQ ID NO: 22. In some embodiments, the variable heavy chain domain of the antibody or fragment thereof is the amino acid sequence of SEQ ID NO: 23, and the variable light chain domain of the antibody or fragment thereof is the amino acid sequence of SEQ ID NO: 6. In some embodiments, the antibody or fragment thereof is configured to bind to a hemagglutinin (HA) protein of an H7N9 influenza virus. In some embodiments, the antibody or fragment thereof is configured to neutralize the H7N9 influenza virus. In some embodiments, the antibody or fragment thereof is configured to bind to a globular head domain (HA1) of a hemagglutinin (HA) protein of an H7N9 influenza virus. In some embodiments, the antibody or fragment thereof is configured to bind to an overlapping region of an antigenic site D of the globular head domain (HA1). In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding. In some embodiments, the antibody or fragment thereof is configured to bind to a stalk region (HA2) of a hemagglutinin (HA) protein of an H7N9 influenza virus. In some embodiments, described herein is a host cell comprising a vector described herein.

In another aspect, described herein is a method of making an antibody or fragment thereof comprising: culturing a host cell under conditions suitable for an expression of a vector, wherein the vector comprises at least one polynucleotide sequence encoding the antibody or fragment thereof, wherein the antibody or fragment thereof comprises a variable heavy chain domain and a variable light chain domain, wherein the variable heavy chain domain of the antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22; and recovering the antibody or fragment thereof. In some embodiments, the variable heavy chain domain of the antibody or fragment thereof comprises the amino acid sequence of SEQ ID NO: 1, and the variable light chain domain of the antibody or fragment thereof comprises the amino acid sequence of SEQ ID NO: 17. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 19. In some embodiments, the variable heavy chain domain of the antibody or fragment thereof is the amino acid sequence of SEQ ID NO: 1, and the variable light chain domain of the antibody or fragment thereof is the amino acid sequence of SEQ ID NO: 21. In some embodiments, the variable heavy chain domain of the antibody or fragment thereof comprises the amino acid sequence of SEQ ID NO: 3, and the variable light chain domain of the antibody or fragment thereof comprises the amino acid sequence of SEQ ID NO: 18. In some embodiments, the variable heavy chain domain of the antibody or fragment thereof is the amino acid sequence of SEQ ID NO: 3, and the variable light chain domain of the antibody or fragment thereof is the amino acid sequence of SEQ ID NO: 20. In some embodiments, the variable heavy chain domain of the antibody or fragment thereof is the amino acid sequence of SEQ ID NO: 3, and the variable light chain domain of the antibody or fragment thereof is the amino acid sequence of SEQ ID NO: 22. In some embodiments, the variable heavy chain domain of the antibody or fragment thereof comprises the amino acid sequence of SEQ ID NO: 23, and the variable light chain domain of the antibody or fragment thereof comprises the amino acid sequence of SEQ ID NO: 6. In some embodiments, the antibody or fragment thereof is configured to bind to a hemagglutinin (HA) protein of an H7N9 influenza virus. In some embodiments, the antibody or fragment thereof is configured to neutralize the H7N9 virus. In some embodiments, the antibody or fragment thereof is configured to bind to a globular head domain (HA1) of a hemagglutinin (HA) protein of an H7N9 virus. In some embodiments, the antibody or fragment thereof is configured to bind to a portion overlapping with an antigenic site D of the globular head domain (HA1). In some embodiments, the antibody or fragment thereof is configured to bind to a 14-centered surface of a lateral patch of the globular head domain (HA1) of the H7N9 influenza virus. In some embodiments, the antibody or fragment thereof is configured to disrupt a loop of the HA protein required for sialic acid binding. In some embodiments, the antibody or fragment thereof is configured to bind to a stalk region (HA2) of a hemagglutinin (HA) protein of an H7N9 influenza virus.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the synthetic antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the variable heavy chain domain of the synthetic antibody or fragment thereof comprises the amino acid sequence of SEQ ID NO: 1, and the variable light chain domain of the synthetic antibody or fragment thereof comprises the amino acid sequence of SEQ ID NO: 17. In some embodiments, the variable heavy chain domain of the synthetic antibody or fragment thereof is the amino acid sequence of SEQ ID NO: 1, and the variable light chain domain of the synthetic antibody or fragment thereof is the amino acid sequence of SEQ ID NO: 19. In some embodiments, the variable heavy chain domain of the synthetic antibody or fragment thereof is the amino acid sequence of SEQ ID NO: 1, and the variable light chain domain of the synthetic antibody or fragment thereof is the amino acid sequence of SEQ ID NO: 21. In some embodiments, the variable heavy chain domain of the synthetic antibody or fragment thereof comprises the amino acid sequence of SEQ ID NO: 3, and the variable light chain domain of the synthetic antibody or fragment thereof comprises the amino acid sequence of SEQ ID NO: 18. In some embodiments, the variable heavy chain domain of the synthetic antibody or fragment thereof is the amino acid sequence of SEQ ID NO: 3, and the variable light chain domain of the synthetic antibody or fragment thereof is the amino acid sequence of SEQ ID NO: 20. In some embodiments, the variable heavy chain domain of the synthetic antibody or fragment thereof is the amino acid sequence of SEQ ID NO: 3, and the variable light chain domain of the synthetic antibody or fragment thereof is the amino acid sequence of SEQ ID NO: 22. In some embodiments, the variable heavy chain domain of the synthetic antibody or fragment thereof comprises the amino acid sequence of SEQ ID NO: 23, and the variable light chain domain of the synthetic antibody or fragment thereof comprises the amino acid sequence of SEQ ID NO: 6. In some embodiments, the antibody or fragment thereof is configured to bind to a hemagglutinin (HA) protein of an H7N9 influenza virus. In some embodiments, the antibody or fragment thereof is configured to bind to a portion overlapping with an antigenic site D of the globular head domain (HA1). In some embodiments, the antibody or fragment thereof is configured to bind to a 14-centered surface of a lateral patch of the globular head domain (HA1) of the H7N9 influenza virus. In some embodiments, the antibody or fragment thereof is configured to disrupt a loop of the HA protein required for sialic acid binding. In some embodiments, the antibody or fragment thereof is configured to bind to a stalk region (HA2) of a hemagglutinin (HA) protein of an H7N9 virus.

In another aspect, described herein is a method of preventing or treating an infection in a subject caused by an H7N9 influenza virus, the method comprising administering to the subject an effective amount of a pharmaceutical composition, wherein the pharmaceutical composition comprises: a synthetic antibody or fragment thereof comprising a variable heavy chain domain and a variable light chain domain, wherein the variable heavy chain domain of the synthetic antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the synthetic antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the variable heavy chain domain of the first antibody or fragment thereof comprises the amino acid sequence of SEQ ID NO: 1, and the variable light chain domain of the first antibody or fragment thereof comprises the amino acid sequence of SEQ ID NO: 17. In some embodiments, the variable heavy chain domain of the first antibody or fragment thereof is the amino acid sequence of SEQ ID NO: 1, and the variable light chain domain of the first antibody or fragment thereof is the amino acid sequence of SEQ ID NO: 19. In some embodiments, the variable heavy chain domain of the first antibody or fragment thereof is the amino acid sequence of SEQ ID NO: 1, and the variable light chain domain of the first antibody or fragment thereof is the amino acid sequence of SEQ ID NO: 21. In some embodiments, the variable heavy chain domain of the first antibody or fragment thereof comprises the amino acid sequence of SEQ ID NO: 3, and the variable light chain domain of the first antibody or fragment thereof comprises the amino acid sequence of SEQ ID NO: 18. In some embodiments, the variable heavy chain domain of the first antibody or fragment thereof is the amino acid sequence of SEQ ID NO: 3, and the variable light chain domain of the first antibody or fragment thereof is the amino acid sequence of SEQ ID NO: 20. In some embodiments, the variable heavy chain domain of the first antibody or fragment thereof is the amino acid sequence of SEQ ID NO: 3, and the variable light chain domain of the first antibody or fragment thereof is the amino acid sequence of SEQ ID NO: 22. In some embodiments, the variable heavy chain domain of the first antibody or fragment thereof comprises the amino acid sequence of SEQ ID NO: 23, and the variable light chain domain of the first antibody or fragment thereof comprises the amino acid sequence of SEQ ID NO: 6. In some embodiments, the method comprises administering to the subject the effective amount of the pharmaceutical composition to prevent the infection in the subject caused by an H7N9 influenza virus. In some embodiments, the effective amount of the pharmaceutical composition is in the range of about 0.1 mg/kg body weight to about 0.5 mg/kg body weight per dose. In some embodiments, at least one of (i) the first antibody or fragment thereof or (ii) second antibody or fragment thereof is configured to bind to a hemagglutinin (HA) protein of an H7N9 influenza virus. In some embodiments, at least one of (i) the first antibody or fragment thereof or (ii) second antibody or fragment thereof is configured to neutralize the H7N9 virus. In some embodiments, the first antibody or fragment thereof is configured to bind to a globular head domain (HA1) of a hemagglutinin (HA) protein of an H7N9 virus. In some embodiments, the first antibody or fragment thereof is configured to bind to a portion overlapping with an antigenic site D of the globular head domain (HA1). In some embodiments, the first antibody or fragment thereof is configured to bind to a β14-centered surface of a lateral patch of the globular head domain (HA1) of the H7N9 influenza virus. In some embodiments, the first antibody or fragment thereof is configured to disrupt a 220-loop of the HA protein required for sialic acid binding. In some embodiments, the second antibody or fragment thereof is configured to bind to a stalk region (HA2) of a hemagglutinin (HA) protein of an H7N9 influenza virus.

In another aspect, described herein is a means for binding a hemagglutinin (HA) protein of an H7N9 virus. In some embodiments, the means comprises a variable heavy chain domain of an antibody or fragment thereof comprising an amino acid sequence selected from the group consisting of SEQ ID NO: 1, SEQ ID NO: 3, SEQ ID NO: 5, SEQ ID NO: 7, and SEQ ID NO: 23, and a variable light chain domain of the antibody or fragment thereof comprising the amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the means comprises a variable heavy chain domain of an antibody or fragment thereof comprising the amino acid sequence of SEQ ID NO: 1, and a variable light chain domain of the antibody or fragment thereof comprising the amino acid sequence of SEQ ID NO: 17. In some embodiments, the means are a variable heavy chain domain of an antibody or fragment thereof consisting of the amino acid sequence of SEQ ID NO: 1, and a variable light chain domain of the antibody or fragment thereof consisting of the amino acid sequence of SEQ ID NO: 19. In some embodiments, the means are a variable heavy chain domain of an antibody or fragment thereof consisting of the amino acid sequence of SEQ ID NO: 1, and a variable light chain domain of the antibody or fragment thereof consisting of the amino acid sequence of SEQ ID NO: 21. In some embodiments, the means comprises a variable heavy chain domain of an antibody or fragment thereof comprising the amino acid sequence of SEQ ID NO: 3, and a variable light chain domain of the antibody or fragment thereof comprising the amino acid sequence of SEQ ID NO: 18. In some embodiments, the means consisting of a variable heavy chain domain of an antibody or fragment thereof comprising the amino acid sequence of SEQ ID NO: 3, and a variable light chain domain of the antibody or fragment thereof consisting of the amino acid sequence of SEQ ID NO: 20. In some embodiments, the means consisting of a variable heavy chain domain of an antibody or fragment thereof comprising the amino acid sequence of SEQ ID NO: 3, and a variable light chain domain of the antibody or fragment thereof consisting of the amino acid sequence of SEQ ID NO: 22. In some embodiments, the means comprises a variable heavy chain domain of an antibody or fragment thereof comprising the amino acid sequence of SEQ ID NO: 23, and a variable light chain domain of the antibody or fragment thereof comprising the amino acid sequence of SEQ ID NO: 6. In some embodiments, the means is configured to bind to a portion overlapping with an antigenic site D of a globular head domain (HA1) of the HA protein of the H7N9 virus. In some embodiments, the means is configured to bind to a β14-centered surface of a lateral patch of the globular head domain (HA1) of the H7N9 influenza virus. In some embodiments, the means is configured to disrupt a 220-loop of the HA protein required for sialic acid binding. In some embodiments, the means is configured to bind to a stalk region (HA2) of the HA protein of an H7N9 virus.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 1, SEQ ID NO: 3, SEQ ID NO: 5, SEQ ID NO: 7, or SEQ ID NO: 23, and the variable light chain domain of the synthetic antibody or fragment thereof comprises an amino acid sequence that is at least 80% identical to SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, or SEQ ID NO: 22. In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 17. In some embodiments, the variable heavy chain domain of the synthetic antibody or fragment thereof is the amino acid sequence that is at least 80% identical to SEQ ID NO: 1, and the variable light chain domain of the synthetic antibody or fragment thereof is the amino acid sequence that is at least 80% identical to SEQ ID NO: 19. In some embodiments, the variable heavy chain domain of the synthetic antibody or fragment thereof is the amino acid sequence that is at least 80% identical to SEQ ID NO: 1, and the variable light chain domain of the synthetic antibody or fragment thereof is the amino acid sequence that is at least 80% identical to SEQ ID NO: 21. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 18. In some embodiments, the variable heavy chain domain of the synthetic antibody or fragment thereof is the amino acid sequence that is at least 80% identical to SEQ ID NO: 3, and the variable light chain domain of the synthetic antibody or fragment thereof is the amino acid sequence that is at least 80% identical to SEQ ID NO: 20. In some embodiments, the variable heavy chain domain of the synthetic antibody or fragment thereof is the amino acid sequence that is at least 80% identical to SEQ ID NO: 3, and the variable light chain domain of the synthetic antibody or fragment thereof is the amino acid sequence that is at least 80% identical to SEQ ID NO: 22. In some embodiments, the variable heavy chain domain of the synthetic antibody or fragment thereof comprises the amino acid sequence that is at least 80% identical to SEQ ID NO: 23, and the variable light chain domain of the synthetic antibody or fragment thereof comprises the amino acid sequence that is at least 80% identical to SEQ ID NO: 6. In some embodiments, the synthetic antibody or fragment thereof is configured to bind to a hemagglutinin (HA) protein of an H7N9 influenza virus. In some embodiments, the synthetic antibody or fragment thereof is configured to bind to a portion overlapping with an antigenic site D of the globular head domain (HA1). In some embodiments, the synthetic antibody or fragment thereof is configured to bind to a β14-centered surface of a lateral patch of the globular head domain (HA1) of the H7N9 influenza virus. In some embodiments, the synthetic antibody or fragment thereof is configured to disrupt a 220-loop of the HA protein required for sialic acid binding. In some embodiments, the synthetic antibody or fragment thereof is configured to bind to a stalk region (HA2) of a hemagglutinin (HA) protein of an H7N9 virus. In some embodiments, a structure of the synthetic antibody or fragment thereof is substantially similar to a structure of a reference antibody or fragment thereof, wherein the reference antibody or structure thereof comprises a variable heavy chain domain and a variable light chain domain, wherein the variable heavy chain domain of the reference antibody or fragment thereof comprises an amino acid sequence selected from the group consisting of SEQ ID NO: 1, SEQ ID NO: 3, SEQ ID NO: 5, SEQ ID NO: 7, and SEQ ID NO: 23, and the variable light chain domain of the reference antibody or fragment thereof comprises an amino acid sequence selected from the group consisting of SEQ ID NO: 2, SEQ ID NO: 4, SEQ ID NO: 6, SEQ ID NO: 8, SEQ ID NO: 17, SEQ ID NO: 18, SEQ ID NO: 19, SEQ ID NO: 20, SEQ ID NO: 21, and SEQ ID NO: 22. In some embodiments, the structure of the reference antibody or fragment thereof is determined by a structure prediction method. In some embodiments, the structure of the synthetic antibody or fragment thereof is determined by a structure prediction method. In some embodiments, the structure prediction method is AlphaFold or RaptorX. In some embodiments, the structure of the reference antibody or fragment thereof and the structure of the synthetic antibody or fragment thereof are further determined by X-ray crystallography or cryo-EM.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 1, SEQ ID NO: 3, SEQ ID NO: 5, SEQ ID NO: 7, and the variable light chain domain of the synthetic antibody or fragment thereof comprises an amino acid sequence that is at least 80% identical to SEQ ID NO: 17, SEQ ID NO: 18, SEQ ID NO: 19, SEQ ID NO: 20, SEQ ID NO: 21, or SEQ ID NO: 22.

In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 17. In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 19. In some embodiments, the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 21. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 18. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 20. In some embodiments, the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 22. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding. In some embodiments, the synthetic antibody is configured to neutralize an H7N9 influenza virus.

In another aspect, described herein is a synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 23 and the variable light chain domain of the synthetic antibody or fragment thereof comprises an amino acid sequence that is at least 80% identical to SEQ ID NO: 2, SEQ ID NO: 4, SEQ ID NO: 6, or SEQ ID NO: 8.

In some embodiments, the variable light chain domain of the synthetic antibody or fragment thereof comprises an amino acid sequence that is at least 80% identical to SEQ ID NO: 6. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding.

In another aspect, described herein is a synthetic antibody or fragment thereof comprising a variable heavy chain domain and a variable light chain domain, wherein the variable heavy chain domain of the synthetic antibody or fragment thereof comprises the complementarity-determining regions (CDRs) of SEQ ID NO: 1, SEQ ID NO: 3, SEQ ID NO: 5, or SEQ ID NO: 7 and the variable light chain domain of the synthetic antibody or fragment thereof comprises the CDRs of SEQ ID NO: 17, SEQ ID NO: 18, SEQ ID NO: 19, SEQ ID NO: 20, SEQ ID NO: 21, or SEQ ID NO: 22.

In some embodiments, the variable heavy chain domain of the synthetic antibody or fragment thereof comprises the CDRs of SEQ ID NO: 1, and the variable light chain domain of the synthetic antibody or fragment thereof comprises the CDRs of SEQ ID NO: 17. In some embodiments, the variable heavy chain domain of the synthetic antibody or fragment thereof comprises the CDRs of SEQ ID NO: 1, and the variable light chain domain of the synthetic antibody or fragment thereof comprises the CDRs of SEQ ID NO: 19. In some embodiments, the variable heavy chain domain of the synthetic antibody or fragment thereof comprises the CDRs of SEQ ID NO: 1, and the variable light chain domain of the synthetic antibody or fragment thereof comprises the CDRs of SEQ ID NO: 21. In some embodiments, the variable heavy chain domain of the synthetic antibody or fragment thereof comprises the CDRs of SEQ ID NO: 3, and the variable light chain domain of the synthetic antibody or fragment thereof comprises the CDRs of SEQ ID NO: 18. In some embodiments, the variable heavy chain domain of the synthetic antibody or fragment thereof comprises the CDRs of SEQ ID NO: 3, and the variable light chain domain of the synthetic antibody or fragment thereof comprises the CDRs of SEQ ID NO: 20. In some embodiments, the variable heavy chain domain of the synthetic antibody or fragment thereof comprises the CDRs of SEQ ID NO: 3, and the variable light chain domain of the synthetic antibody or fragment thereof comprises the CDRs of SEQ ID NO: 22. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding. In some embodiments, the synthetic antibody is configured to neutralize an H7N9 influenza virus.

In another aspect, described herein is a synthetic antibody or fragment thereof comprising a variable heavy chain domain and a variable light chain domain, wherein the variable heavy chain domain of the synthetic antibody or fragment thereof comprises the CDRs of SEQ ID NO: 23 and the variable light chain domain of the synthetic antibody or fragment thereof comprises the CDRs of SEQ ID NO: 2, SEQ ID NO: 4, SEQ ID NO: 6, or SEQ ID NO: 8.

In some embodiments, the variable light chain domain of the synthetic antibody or fragment thereof comprises the CDRs of SEQ ID NO: 6. In some embodiments, the antibody or fragment thereof is configured to bind to a hemagglutinin (HA) protein of an H7N9 influenza virus. In some embodiments, the antibody or fragment thereof is configured to bind to a region overlapping with an antigenic site D of the globular head domain (HA1) of the H7N9 influenza virus. In some embodiments, the antibody or fragment thereof is configured to bind to a β14-centered surface of a lateral patch of the globular head domain (HA1) of the H7N9 influenza virus. In some embodiments, the antibody or fragment thereof is configured to disrupt a 220-loop of the HA protein required for sialic acid binding.

In another aspect, described herein is a polynucleotide encoding a synthetic antibody described herein.

In another aspect, described herein is a method of preventing or treating an infection in a subject caused by an H7N9 influenza virus, the method comprising administering to the subject an effective amount of a pharmaceutical composition, wherein the pharmaceutical composition comprises a synthetic antibody described herein.

In another aspect, described herein is a method of preventing or treating an infection in a subject caused by an H7N9 influenza virus, the method comprising administering to the subject an effective amount of a pharmaceutical composition, wherein the pharmaceutical composition comprises a polynucleotide described herein.

### Compositions

In some embodiments, a prophylactic or therapeutic composition of this disclosure comprises one or more antibodies (or one or more polynucleotides encoding one or more antibodies) and is administered in a pharmaceutical composition that includes a pharmaceutically acceptable carrier. In some embodiments, the prophylactic or therapeutic composition is comprised of one or more antibodies (or one or more polynucleotides encoding one or more antibodies) comprising SEQ ID NOs 1, 2, 3, 4, 5, 6, 7, 8, 17, 18, 19, 20, 21, 22, and/or 23 (e.g., antibody “H7.HK1” comprising SEQ ID NOs: 1 and 2, “H7.HK2” comprising SEQ ID NOs: 3 and 4, “H7.HK3” comprising SEQ ID NOs: 5 and 6, and/or “H7.HK4” comprising SEQ ID NOs: 7 and 8). In some embodiments, the prophylactic or therapeutic composition is comprised of one or more antibodies (or one or more polynucleotides encoding one or more antibodies) comprising N-linked glycan removal mutations for mAbs H7.HK1, H7.HK2, and H7.HK3, for example, “H7.HK1 K_S26A” (SEQ ID NO: 1 and SEQ ID NO: 17), “H7.HK1 K_N24Q” (SEQ ID NO: 1 and SEQ ID NO: 19), “H7.HK1 K_N24R” (SEQ ID NO: 1 and SEQ ID NO: 21), “H7.HK2 K_S28A” (SEQ ID NO: 3 and SEQ ID NO: 18), “H7.HK2 K_N26Q” (SEQ ID NO: 3 and SEQ ID NO: 20), “H7.HK2 K_N26S” (SEQ ID NO: 3 and SEQ ID NO: 22), and/or “H7.HK3 H_S54A” (SEQ ID NO: 23 and SEQ ID NO: 6). In some embodiments, the pharmaceutical composition is in the form of a spray, aerosol, gel, solution, emulsion, nanoparticle (e.g., lipid nanoparticle), or suspension.

The composition is preferably administered to a subject with a pharmaceutically acceptable carrier. Typically, in some embodiments, an appropriate amount of a pharmaceutically acceptable salt is used in the formulation, which in some embodiments can render the formulation isotonic.

In certain embodiments, the one or more antibodies (or one or more polynucleotides encoding one or more antibodies) are provided as a composition comprising any one of the antibodies described herein (“H7.HK1” comprising SEQ ID NOs: 1 and 2, “H7.HK2” comprising SEQ ID NOs: 3 and 4, “H7.HK3” comprising SEQ ID NOs: 5 and 6, and/or “H7.HK4” comprising SEQ ID NOs: 7 and 8) and a pharmaceutically acceptable carrier. In certain embodiments, the composition further comprises an adjuvant. In certain embodiments, the antibodies are conjugated with other molecules to increase their effectiveness as is known by those practiced in the art.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of antibodies being administered.

The phrase pharmaceutically acceptable carrier as used herein means a pharmaceutically acceptable material, composition, or vehicle, such as a liquid or solid filler, diluent, excipient, solvent or encapsulating material, involved in carrying or transporting the subject pharmaceutical agent from one organ, or portion of the body, to another organ, or portion of the body. Each carrier is acceptable in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of an antibody composition, such as a buffer, a reducing agent, a bulk protein, amino acids (such as e.g., glycine or praline) or a carbohydrate. Typical carbohydrates useful in formulating compositions include but are not limited to sucrose, mannitol, lactose, trehalose, or glucose.

Surfactants may also be used to prevent soluble and insoluble aggregation and/or precipitation of antibodies included in the composition. Suitable surfactants include but are not limited to sorbitan trioleate, soya lecithin, and oleic acid. In certain cases, solution aerosols are preferred using solvents such as ethanol. Thus, formulations including antibodies can also include a surfactant that can reduce or prevent surface-induced aggregation of antibodies by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. In some embodiments, surfactants used with the present disclosure are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called stabilizers or preservatives may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing antibodies at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., antibodies) as an active ingredient.

A liquid composition herein can be used as such with a delivery device, or they can be used for the preparation of pharmaceutically acceptable formulations comprising antibodies that are prepared for example by the method of spray drying. The methods of spray freeze-drying proteins for pharmaceutical administration disclosed in Maa et al., Curr. Pharm. Biotechnol., 2001, 1, 283-302, are incorporated by reference herein. In another embodiment, the liquid solutions herein are freeze spray dried and the spray-dried product is collected as a dispersible peptide-containing powder that is therapeutically effective when administered to an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures (e.g., antibodies can be used in combination treatment with another treatment such as antivirals or with a vaccine, and/or another treatment). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The current invention provides for dosage forms comprising peptides suitable for treating cancer or other diseases. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999), which is hereby incorporated by reference in its entirety.

Generally, a dosage form used in the acute treatment of a disease may contain larger amounts of one or more of the active ingredients it comprises than a dosage form used in the chronic treatment of the same disease. In addition, the prophylactically and therapeutically effective dosage form may vary among different conditions. For example, a therapeutically effective dosage form may contain one or more antibodies that have an appropriate therapeutic action when intending to treat infection caused by influenza virus (e.g., H7N9 influenza). On the other hand, a different effective dosage may contain one or more antibodies that have an appropriate prophylactic action when intending to prevent infection caused by influenza virus (e.g., H7N9 influenza). These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, each of which is incorporated herein by reference in its entirety.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

Compositions can be formulated with appropriate carriers and adjuvants using techniques to yield compositions suitable for prophylaxis or treatment. The compositions can include an adjuvant, such as, for example but not limited to, alum, poly IC, MF-59, squalene-based adjuvants, or liposomal based adjuvants suitable for prophylaxis or treatment.

In some embodiments, the antibodies described herein are encoded by nucleic acids which are prepared in a mRNA-LNP or a DNA-LNP formulation for administration to a subject.

### Antibody Production

The antibodies disclosed herein can be produced by any method known in the art. In some embodiments, the antibodies disclosed herein are produced by culturing a cell transfected or transformed with a vector comprising nucleic acid sequences encoding an antibody described herein and isolating the antibody.

In some embodiments, mAbs are synthesized by the hybridoma culture method which results in mAbs that are not contaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques known in the art, including, for example, the hybridoma method (e.g., Kohler and Milstein., Nature, 256:495-97 (1975); Hongo et al., Hybridoma, 14 (3): 253-260 (1995), Harlow et al, Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N. Y., 1981)), recombinant DNA methods, phage-display technologies (see, e.g., Clackson et al., Nature, 352:624-628 (1991); Marks et al., J. Mol Biol. 222:581-597 (1992); Sidhu et al., J. Mol Biol. 338 (2): 299-310 (2004); Lee et al., J. Mol Biol. 340 (5): 1073-1093 (2004); Fellouse, Proc. Natl. Acad. ScL USA 101 (34): 12467-12472 (2004); and Lee et al., J. Immunol. Methods 284 (1-2): 119-132 (2004), and technologies for producing human or humanlike antibodies in animals that have parts or all of the human immunoglobulin loci or genes encoding human immunoglobulin sequences (see, e.g., Lonberg et al., Nature 368:856-859 (1994); Morrison, Nature 368:812-813 (1994); Fishwild et al., Nature Biotechnol 14:845-851 (1996); Neuberger, Nature Biotechnol. 14:826 (1996); and Lonberg and Huszar, Intern. Rev. Immunol. 13:65-93 (1995), all of which are incorporated by reference.

In some embodiments, expression of an antibody comprises expression vector(s) containing a polynucleotide that encodes the anti-H7N9 antibody. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Particular embodiments provide replicable vectors comprising a nucleotide sequence encoding an anti-H7N9 antibody disclosed herein operably linked to a promoter. In preferred embodiments, such vectors may include a nucleotide sequence encoding the heavy chain of an antibody molecule (or fragment thereof), a nucleotide sequence encoding the light chain of an antibody (or fragment thereof), or both the heavy and light chain.

The polynucleotide encoding the antibody may be modified, for example, by substituting the coding sequence for human heavy- and light-chain constant domains in place of the homologous murine sequences (U.S. Pat. No. 4,816,567; Morrison, et al., Proc. Natl Acad. ScL USA, 81:6851 (1984), each of which is incorporated by reference herein in its entirety), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. The monoclonal antibodies described herein may be monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies disclosed herein in order to produce the antibody of interest. Expression vectors containing such a nucleic acid sequence, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign antibody expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56 (2): 221-234) or can be determined by the skilled artisan (see, for example, Animal Cell Culture: A Practical Approach 2nd Ed., Rickwood, D. and Hames, B. D., eds. (Oxford University Press: New York, 1992), each of which is incorporated by reference herein in its entirety). Cell culturing conditions can vary according to the type of host cell selected. Commercially available media can be utilized.

Monoclonal antibodies disclosed herein (e.g., comprising one of more of SEQ ID NOs: 1, 2, 3, 4, 5, 6, 7, and 8) can be purified from any human or non-human cell which expresses the antibody, including those which have been transfected with expression constructs that express the antibody. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Methods of Treatment

In one embodiment, the subject matter disclosed herein relates to a preventive medical treatment started after following diagnosis of H7N9 infection to prevent the disease from worsening or curing the disease. In one embodiment, the subject matter disclosed herein relates to prophylaxis of subjects who are believed to be at risk for moderate or severe disease associated with H7N9 infection or have previously been diagnosed with another disease. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method known in the art.

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; (g) administration through implantation; and administration via electroporation.

In some embodiments, one or more antibodies disclosed herein are prepared in a cocktail of DNA-encoding mAbs or mRNA-encoding mAbs and delivered by electroporation to a subject for in vivo expression of the encoded mAbs.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7 (2): 27.)

### Methods of Prevention

In some embodiments, the compositions prepared using methods of the invention can be used as a vaccine to promote an immune response against future H7N9 infection. In some embodiments, the antibodies are neutralizing antibodies.

In some embodiments, the antibodies (or polynucleotides encoding antibodies) prepared using methods of the invention can be combined with additional vaccine components.

### Dosage

A prophylactically effective or therapeutically effective amount is typically dependent on the weight of the subject being treated, the subject's physical condition, the extensiveness of the condition to be treated, and the age of the subject being treated. In general, anti-H7N9 antibodies, or polynucleotides encoding one or more antibodies, disclosed herein may be administered in an amount in the range of about 10 ng/kg body weight to about 100 mg/kg body weight per dose. In some embodiments, antibodies may be administered in an amount in the range of about 10 μg/kg body weight to about 30 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 1 mg/kg body weight to about 5 mg/kg body weight per dose. In some embodiments, antibodies may be administered in an amount in the range of about 1 mg/kg body weight to about 20 mg/kg body weight per dose. In some embodiments, antibodies may be administered in an amount in the range of about 5 mg/kg body weight to about 15 mg/kg body weight per dose. In some embodiments, antibodies may be administered in an amount of about 10 mg/kg body weight per dose. In some embodiments, antibodies may be administered in an amount in the range of about 10 mg/kg body weight per dose to 50 mg/kg body weight. In some embodiments, antibodies may be administered in an amount in the range of about 0.1 mg/kg body weight to about 0.5 mg/kg body weight per dose. In some embodiments, antibodies may be administered in a dose of at least about 100 μg/kg body weight, at least about 250 μg/kg body weight, at least about 500 μg/kg body weight, at least about 750 μg/kg body weight, at least about 3 mg/kg body weight, at least about 5 mg/kg body weight, at least about 10 mg/kg body weight, at least about 15 mg/kg body weight, or at least about 20 mg/kg body weight.

In some methods, the dosage is adjusted to achieve a plasma antibody concentration of about 1-1000 μg/mL or about 25-300 μg/mL. In some embodiments, the dosage is adjusted to achieve a plasma antibody concentration of about 0.001 μg/mL to about 10 μg/mL. In some embodiments, the dosage is adjusted to achieve a plasma antibody concentration of about 0.01 μg/mL to about 1 μg/mL. In some embodiments, the dosage is adjusted to achieve a plasma antibody concentration of about 0.01 μg/mL to about 0.1 μg/mL.

### EXAMPLES

### Example 1: Study Design

This study was initiated by a confirmed 2013 H7N9 infection case in Hong Kong with the virus isolated and plasma neutralizing antibodies evident (K. K. To, et al., Unique reassortant of influenza A (H7N9) virus associated with severe disease emerging in Hong Kong. The Journal of infection 69, 60-68 (2014), incorporated by reference herein in its entirety). The goal of the study was to isolate and functionally characterize human mAbs for antiviral activities against H7N9. The patient PBMCs were collected one year post recovery, and an H7 HA probe was devised to sort the H7-reactive memory B cells and recover human mAbs. ELISA was used to confirm the mAb binding specificity to the H7 HA antigen, and neutralization assays were used to assess the mAbs for H7N9 neutralization activity. Cryo-EM was carried out to structurally determine the mAb epitopes and mechanism of neutralization. The mAb antiviral prophylactic and therapeutic effectiveness was assessed in a mouse lethal challenge model. From the isolated mAbs, not only was potent H7N9 neutralization demonstrated, but cryo-EM analysis was also used to delineate a conserved lateral site-of-vulnerability in the HA head, a finding of vaccine relevance.

### Example 2: Collection of Human Specimens

A blood specimen was collected from the H7N9_HK2013 patient about one year after recovery from a hospitalized severe H7N9 infection. Written informed consent was obtained from the patient. Each specimen is unique and cannot be replaced once processed. The study was approved by the Institutional Review Board (IRB) of the University of Hong Kong and the Hospital Authority (Reference number: UW-13-265).

### Example 3: Plasmids, Viruses, Antibodies, and Cells

Expression plasmids encoding the H7 hemagglutinin (HA) and N9 neuraminidase based on A/Shanghai/4664T/2013 H7N9 strain were obtained from Dr. Jianqing Xu (see Qiu C, et al. Safe pseudovirus-based assay for neutralization antibodies against influenza A (H7N9) virus. Emerg Infect Dis 19:1685-7, which is hereby incorporated by reference in its entirety). Codon-optimized gene encoding the H7 hemagglutinin of A/Hong Kong/125/2017 H7N9 was synthesized (Twist Bioscience) and cloned into pcDNA3.1 (Invitrogen). HIV-1 pNL4-3 Aenv.Luc.R-E-backbone was obtained through the NIH HIV Reagent Program. These plasmids were used to co-transfect 293T cells to generate the H7N9 pseudo viruses. All live replicating influenza A viruses used in this study were isolated from patients and include A/Hong Kong/470129/2013 H7N9 (K. K. To, et al., Unique reassortant of influenza A (H7N9) virus associated with severe disease emerging in Hong Kong. The Journal of infection 69, 60-68 (2014), incorporated by reference herein in its entirety), A/Zhejiang/DTID-ZJU01/2013 H7N9 (Y. Chen, et al., Human infections with the emerging avian influenza A H7N9 virus from wet market poultry: clinical analysis and characterization of viral genome. Lancet 381, 1916-1925 (2013), incorporated by reference herein in its entirety), A/Anhui/1/2013 H7N9 (obtained from the China Center for Disease Control and Prevention), A/Vietnam/1194/2004 H5N1, A/Hong Kong/459094/2010 H5N1, A/Hong Kong/1073/1999 H9N2, A/Hong Kong/415742/2009 H1N1, and A/Hong Kong/400500/2015 H3N2. DNA sequences encoding the variable regions of previous H7N9 mAbs L4A-14 (PDB: 6114), H7.167 (PDB: 5V2A), and 07-5F01 (GenBank KU987563 and KU987564) were synthesized (Twist Bioscience) and cloned into the corresponding human gamma, kappa, and lambda chain expression vectors described (see Tiller T, et al. Efficient generation of monoclonal antibodies from single human B cells by single cell RT-PCR and expression vector cloning. J Immunol Methods 329, 112-124 (2008); Yang Z Y, et al. Rational design of envelope identifies broadly neutralizing human monoclonal antibodies to HIV-1. Science 329, 856-861 (2010); M. Jia, H. et al., Development of Broadly Neutralizing Antibodies and Their Mapping by Monomeric gp120 in Human Immunodeficiency Virus Type 1-Infected Humans and Simian-Human Immunodeficiency Virus SHIVSF162P3NInfected Macaques. Journal of virology 90, 4017-4031 (2016), each of which is hereby incorporated by reference in its entirety). Antibody point mutations (e.g., see Example 21 and FIGS. 23 and 24) were generated by TagMaster Site-Directed Mutagenesis Kit (GM Biosciences, Frederick, MD). Full IgG1 was expressed by co-transfecting Expi293F cells with equal amounts of paired heavy and light chain plasmids and purified using recombinant Protein A agarose (Thermo Fisher). The non-H7N9 placebo mAb used in this study, AD358_nl, has been described (M. Jia, H. et al., Development of Broadly Neutralizing Antibodies and Their Mapping by Monomeric gp120 in Human Immunodeficiency Virus Type 1-Infected Humans and Simian-Human Immunodeficiency Virus SHIVSF162P3N Infected Macaques. Journal of virology 90, 4017-4031 (2016), incorporated by reference herein in its entirety) and is specific to HIV-1 gp120. Human embryonic kidney 293 cell line, of which the sex is female, is the parental cell for 293T and Expi293F cell lines. 293T was obtained from ATCC (Cat. No. CRL-11268, Clone 17) and maintained as adherent cells in complete DMEM medium at 37° C. 293T is highly transfectable and contains SV40 T-antigen. Expi293F was obtained from ThermoFisher (Cat. No. A14527) and adapted to suspension culture in Expi293 Expression Medium at 37° C. The Madin-Darby Canine Kidney (MDCK) cell line, of which the sex is female, was obtained from ATCC (Cat. No. CCL-34) and maintained as adherent cells in complete DMEM medium at 37° C.

### Example 4: Single B Cell Sorting by Fluorescence Activated Cell Sorter (FACS)

A soluble recombinant HA antigen based on A/Shanghai/2/2013 H7N9 (Immune Technologies, New York, NY) was biotinylated via EZ-Link (Thermo Fisher), followed by streptavidin mediated conjugation of phycoerythrin (PE) (Invitrogen). PBMCs were stained with an antibody cocktail to CD3-PE-CF594 (BD Biosciences, San Jose, CA), CD19-PE-Cy7 (BioLegend, San Diego, CA), CD20-APC-Cy7 (BioLegend), IgG-FITC (BD Biosciences), and IgM-V450 (BD Biosciences). All staining antibodies were purchased from vendors providing Quality Certificates and further validated on healthy human blood donor PBMCs purchased from New York Blood Center. In addition, live/dead yellow stain (Invitrogen) was used to exclude dead cells. After washing, H7-PE⁺ B cells were sorted using a multi-laser MoFlo sorter (Beckman Coulter, Jersey City, NJ). Fluorescence compensation was performed with anti-mouse Ig kappa CompBeads (BD Biosciences) stained with each antibody in a separate tube. Individual B cells were sorted into a 96-well PCR plate, each well containing 20 μL lysis buffer, composed of 0.5 μL RNaseOut (Invitrogen), 5 μL 5× first-strand buffer, 1.25 μL 0.1M DTT, and 0.0625 μL Igepal (Sigma, St. Louis, MO). The PCR plate with sorted cells was frozen on dry-ice and then stored at −80° C. The total cell sample passing through the sorter was analyzed with FlowJo 10.0 (TreeStar, Cupertino, CA).

### Example 5: Single B Cell RT-PCR, Sequencing, and Cloning

From each sorted cell, the variable regions of IgG heavy and light chains were amplified by RT-PCR and cloned into expression vectors as previously described (M. Jia, et al., Development of Broadly Neutralizing Antibodies and Their Mapping by Monomeric gp120 in Human Immunodeficiency Virus Type 1-Infected Humans and Simian-Human Immunodeficiency Virus SHIV_{SF162P3N}-Infected Macaques. Journal of virology 90, 4017-4031 (2016), incorporated by reference herein in its entirety). Briefly, frozen plates with single B cell RNA were thawed at room temperature, and RT was carried out by adding into each well 3 μL random hexamers at 150 ng/μL (Gene Link, Hawthorne, NY), 2 μL dNTP (each at 10 mM), and 1 μL SuperScript II (Invitrogen), followed by incubation at 42° C. for 2 hours. After RT, 25 μL water was added to each well to dilute cDNA, and the cDNA plates were stored at −20° C. for later use. The variable regions of heavy, kappa, and lambda chains were amplified independently by nested PCR in 50 μL, using 5 μL cDNA as template, with HotStarTaq Plus DNA polymerase (Qiagen) and primer mixes as described (see Tiller T, et al. Efficient generation of monoclonal antibodies from single human B cells by single cell RT-PCR and expression vector cloning. J Immunol Methods 329, 112-124 (2008); Scheid J F, et al. 2011. Sequence and structural convergence of broad and potent HIV antibodies that mimic CD4 binding. Science 333, 1633-1637 (2011): 1633-7; each of which is hereby incorporated by reference in its entirety). Cycler parameters were 94° C. for 5 min, 50 cycles of 94° C. for 30 sec, 52-55° C. for 30 sec, and 72° C. for 1 min, followed by 72° C. for 10 min. The PCR amplicons were subjected to direct Sanger sequencing, and the antibody sequences were analyzed using IMGT/V-QUEST. Selected PCR sequences that gave productive gamma, kappa, and lambda chain rearrangements were re-amplified with custom primers containing unique restriction digest sites and cloned into the corresponding human gamma, kappa, and lambda chain expression vectors as described (M. Jia, et al., Development of Broadly Neutralizing Antibodies and Their Mapping by Monomeric gp120 in Human Immunodeficiency Virus Type 1-Infected Humans and Simian-Human Immunodeficiency Virus SHIV_{SF162P3N}-Infected Macaques. Journal of virology 90, 4017-4031 (2016); T. Tiller, et al., Efficient generation of monoclonal antibodies from single human B cells by single cell RT-PCR and expression vector cloning. J Immunol Methods 329, 112-124 (2008); and X. Wu, et al., Rational design of envelope identifies broadly neutralizing human monoclonal antibodies to HIV-1. Science 329, 856-861 (2010), each of which is incorporated by reference herein in its entirety). Full IgG1 was expressed by co-transfecting Expi293F cells (ThermoFisher) with equal amounts of paired heavy and light chain plasmids and purified using recombinant Protein A agarose (ThermoFisher).

### Example 6: ELISA and Competition ELISA

The H7N9 ΔTM HA and HA1 based on A/Shanghai/2/2013, HAIs based on A/Guangdong/17SF003/2016, A/Hong Kong/125/2017, and the H7N7 ΔTM HA based on A/Netherlands/219/2003 were purchased from Immune Technologies, New York, NY. Other non-H7 ΔTM HA proteins were purchased from Sino Biological, Chesterbrook, PA. ELISA plates were coated with HA or HA1 antigens at 2 μg/mL in phosphate buffered saline (PBS) overnight at 4° C. For Endo H treatment, the required amount of antigen was diluted in 10× buffer and mixed with 1 μL Endo H (New England BioLabs, Ipswich, MA) for 1 hour at 37° C.; an equal amount of antigen (untreated) was processed under identical condition without Endo H. Both treated and untreated antigens were then diluted in PBS to coat ELISA plates. Coated plates were blocked with 1% BSA (bovine serum albumin) in PBS, followed by incubation with serially diluted mAbs for 1 hour at 37° C. Horseradish peroxidase (HRP)-conjugated goat anti-human IgG Fc (Jackson ImmunoResearch, West Grove, PA) was added at 1:10,000 for 1 hour at 37° C. All ELISA incubation volumes were 100 μL/well except that 200 μL/well was used for blocking. Plates were washed between steps with 0.1% Tween 20 in PBS and developed with 3,3′,5,5′-tetramethylbenzidine (TMB) (Novex, Life Technologies), with 1 M H₂SO₄ as terminator and read at 450 nm. For competitive ELISA,plates were coated with 2 μg/mL of H7N9 ΔTM HA. After blocking, serial dilutions of competing mAbs were added in 50 μL of blocking buffer, followed by addition of 50 μL of biotin labeled H7.HK2 at 100 ng/mL. After incubation at 37° C. for 1 h, the plates were washed and then incubated with 250 ng/mL of streptavidin-HRP (Jackson ImmunoResearch, West Grove, PA) at ambient temperature for 30 minutes before development with TMB as described above. Similar results were independently reproduced at least once.

### Example 7: SDS-PAGE and Western Blot

Cleaved HA protein from A/Shanghai/1/2013 H7N9 (HA1+HA2, cleavage) (Sino Biological, Beijing, China) was added at 1 μg with 4×SDS loading buffer with reduced reagent and heated at 70° C. for 10 m. The protein was separated on NuPAGE 4-12% Bis-Tris gel with MOPS running buffer (Invitrogen) and transferred to PVDF membrane semi-dry with the Bio-Rad trans-blot turbo transfer system. The membrane was blocked in 2% skim milk in PBS-T, followed by incubation with mAb H7.HK2 or H7.HK4 as primary antibody at 1 μg/mL in blocking buffer at 4° C. overnight. HRP-conjugated goat anti-human IgG Fc (Jackson ImmunoResearch, West Grove, PA) was used as secondary antibody at 1:10,000 in blocking buffer at room temperature for 1 h. The immunoreactive band was detected with ECL reagent (ThermoFisher). Similar results were independently reproduced at least once.

### Example 8: H7N9 Neutralization Assays

H7N9 neutralization was first measured with a single-round infection of MDCK cells using pseudo viruses expressing the H7 gene from A/Shanghai/4664T/2013 H7N9 or A/Hong Kong/125/2017 H7N9, and the N9 gene from A/Shanghai/4664T/2013 H7N9, pseudotyped with the HIV-1 NL4-3-LucAenv backbone (see Qiu C, Huang Y, Zhang A, Tian D, Wan Y, Zhang X, Zhang W, Zhang Z, Yuan Z, Hu Y, Zhang X, Xu J. 2013. Safe pseudovirus-based assay for neutralization antibodies against influenza A (H7N9) virus. Emerg Infect Dis 19:1685-7, which is incorporated by reference herein in its entirety). In 96-well plate, 70 μL of antibody-virus mixture were incubated at 37° C. for 1 h in triplicate wells before transferring to pre-seeded MDCK cells, followed by the addition of 35 μL of DEAE-dextran at a final concentration of 10 μg/mL. To keep assay conditions constant, sham medium was used in place of antibody in control wells. Infection levels were determined 2 days later with Bright-Glo luciferase assay system (Promega, Madison, WI). Neutralization curves were fitted by a 5-parameter nonlinear regression built in Prism 9.5.1 (GraphPad Software, La Jolla, CA). The 50% inhibitory titers (IC₅₀s) were reported as the antibody concentrations required to inhibit infection by 50%. H7N9 neutralization was next measured using live replicating influenza viruses to infect MDCK cells as described (K. K. To, et al., High titer and avidity of nonneutralizing antibodies against influenza vaccine antigen are associated with severe influenza. Clinical and vaccine immunology: CVI 19, 1012-1018 (2012), incorporated by reference herein in its entirety). Briefly, serially diluted mAbs were incubated with 100 TCID50 (50% tissue culture infective dose) of an influenza virus at 37° C. for 2 hours, and 100 μL virus-mAb mixture was added to MDCK cells. After 1 hour incubation, the virus-mAb mixture was removed, and minimum-essential medium with 2 μg/mL L-1-tosylamide-2-phenylethylchloromethyl ketone-treated trypsin (TPCK-trypsin) was added to each well. The plates were then incubated for 72 hours, and cytopathic effects were recorded. The mAb concentration that protected 50% of 5 replicate wells from cytopathology was reported as IC₅₀. Similar results were independently reproduced at least once.

### Example 9: H7 HA Production

Soluble, disulfide-stabilized, fully cleaved H7 HA trimers were produced by transient co-transfection of plasmids encoding H7 HA (H7 SH13 DS2 6R) and Furin of Expi293F cells (Life Technologies) using Turbo293 transfection reagent (Speed biosystem). After 5 days at 37° C., culture supernatants were harvested by centrifugation and concentrated 5-fold by Tangential Flow Filtration. The recombinant HA trimer was captured by Ni-NTA (Sigma-Aldrich) through a C-terminal 6×His-tag. The imidazole eluant was combined 1:1 (v/v) with saturated ammonium sulfate, centrifuged at 4° C., and pellet removed. The supernatant was dialyzed against 500 mM NaCl, 50 mM Tris pH 8, and purified by size exclusion chromatography on a Superdex 200 Increase 10/300 GL column (Cytiva, Marlborough, MA).

### Example 10: Human mAb Fab Preparation

Human mAb Fab fragments were produced by digestion of the full IgG antibodies with immobilized Papain (ThermoFisher) equilibrated with 25 mM phosphate, 150 mM NaCl, pH 10, and 2 mM EDTA buffer for 3 hours. The resulting Fabs were purified from the cleaved Fc domain by affinity chromatography using protein A. Fab purity was analyzed by SDS-PAGE. All Fabs were buffer-exchanged into 25 mM phosphate, 150 mM NaCl, pH 7.0 prior to cryo-EM experiments.

### Example 11: Cryo-EM Sample Preparation, Data Collection, and Structure Determination

To determine the structures of H7.HK1 and H7.HK2 with H7 HA trimer, trimer was mixed with the antibody Fab at 1 to 1.2 molar ratio at a final total protein concentration of ˜1 mg/mL and adjusted to a final concentration of 0.005% (w/v) n-Dodecyl β-D-maltoside (DDM) to prevent preferred orientation and aggregation during vitrification. Cryo-EM grids were prepared by applying 3 μL of sample to a freshly glow discharged carbon-coated copper grid (CF 1.2/1.3 300 mesh). The sample was vitrified in liquid ethane using a Vitrobot Mark IV with a wait time of 30s, a blot time of 3s, and a blot force of 0. Cryo-EM data were collected on a Titan Krios operating at 300 keV, equipped with a K3 detector (Gatan) operating in counting mode. Data were acquired using Leginon (A. Cheng, C. Negro, et al., Legninon: New features and applications. Protein Sci 30, 136-150 (2021), incorporated by reference herein in its entirety). The dose was fractionated over 50 raw frames. For all structures, the movie frames were aligned and dose-weighted (see S. Q. Zheng, et al., MotionCor2: anisotropic correction of beam-induced motion for improved cryo-electron microscopy. Nat Methods 14, 331-332 (2017), incorporated by reference herein in its entirety) using cryoSPARC 3.4 (A. Punjani, et al., cryoSPARC: algorithms for rapid unsupervised cryo-EM structure determination. Nat Methods 14, 290-296 (2017), incorporated by reference herein in its entirety); the CTF estimation, particle picking, 2D classifications, ab initio model generation, heterogeneous refinements, homogeneous 3D refinements and non-uniform refinement calculations were carried out using cryoSPARC 3.4 (A. Punjani, et al., cryoSPARC: algorithms for rapid unsupervised cryo-EM structure determination. Nat Methods 14, 290-296 (2017), incorporated by reference herein in its entirety).

### Example 12: Atomic Model Building and Refinement

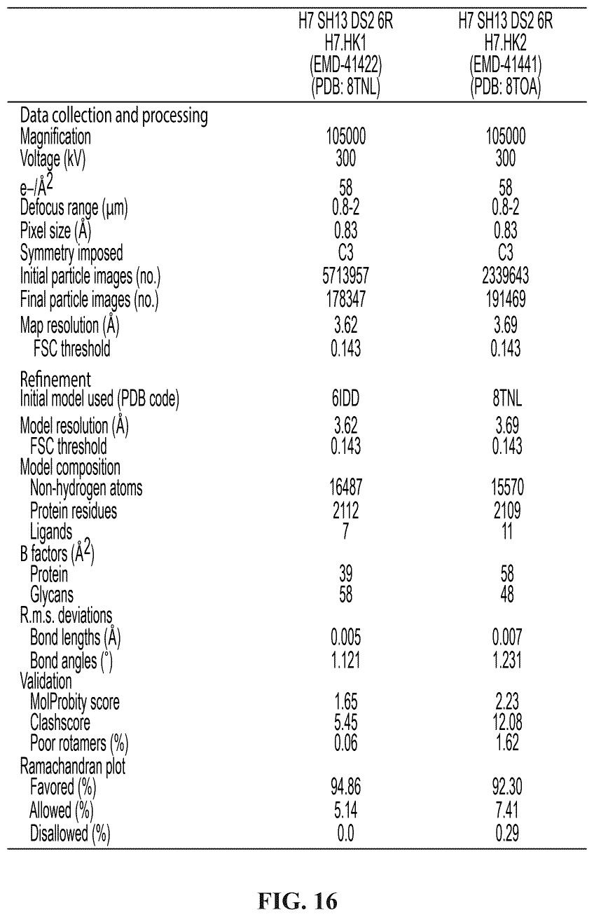

For structural determination, a model of the antibody Fab was generated using SAbPred (J. Dunbar, K. Krawczyk, et al., SAbPred: a structure-based antibody prediction server. Nucleic Acids Res 44, W474-478 (2016), incorporated by reference herein in its entirety). The Fab model and the crystal structure of an H7 HA mutant (PDB 6IDD) (Y. Xu, et al., Avian-to-Human Receptor-Binding Adaptation of Avian H7N9 Influenza Virus Hemagglutinin. Cell Rep 29, 2217-2228 e2215 (2019), incorporated by reference herein in its entirety) was docked into the cryo-EM density map using UCSF Chimera (E. F. Pettersen, et al., UCSF Chimera—a visualization system for exploratory research and analysis. J Comput Chem 25, 1605-1612 (2004), incorporated by reference herein in its entirety) to build an initial model of the complex. The model was then manually rebuilt to the best fit into the density using Coot (A. Casanal, et al., Current developments in Coot for macromolecular model building of Electron Cryo-microscopy and Crystallographic Data. Protein Sci 29, 1069-1078 (2020), incorporated by reference herein in its entirety) and refined using Phenix (P. D. Adams, et al., Recent developments in the PHENIX software for automated crystallographic structure determination. J Synchrotron Radiat 11, 53-55 (2004), incorporated by reference herein in its entirety). Interface calculations were performed using PISA (E. Krissinel, & K. Henrick, Inference of macromolecular assemblies from crystalline state. J Mol Biol 372, 774-797 (2007), incorporated by reference herein in its entirety). Structures were analyzed and figures were generated using PyMOL (http://www.pymol.org) and UCSF Chimera (E. F. Pettersen, et al., UCSF Chimera—a visualization system for exploratory research and analysis. J Comput Chem 25, 1605-1612 (2004), incorporated by reference herein in its entirety). Final model statistics are summarized FIG. 16.

### Example 13: H7 HA Sequence Analysis

Searching the Global Initiative on Sharing All Influenza Data (GISAID) with H7NX returned a total of N=1,511 H7 HA sequences, with N=2 H7N2, N=2 H7N3, N=2 H7N4, N=54 H7N7, and N=1,451 H7N9. After removing N=28 duplicate or defective sequences, N=1,483 H7 amino acid sequences were aligned using Clustal (see Thompson J D, et al. CLUSTAL W: improving the sensitivity of progressive multiple sequence alignment through sequence weighting, position-specific gap penalties and weight matrix choice. Nucleic Acids Res 22:4673-80 (1994), which is incorporated by reference herein in its entirety), and the sequence conservation analysis was performed using AL2CO (see Pei J, et al. 2001. AL2CO: calculation of positional conservation in a protein sequence alignment. Bioinformatics 17:700-12 (2001), which is incorporated by reference herein in its entirety).

### Example 14: Mouse Prophylactic and Therapeutic Studies

The mouse prophylactic and therapeutic studies were approved by the Committee on the Use of Live Animals in Teaching and Research (CULATR) of the University of Hong Kong (Reference number: 4011-16) and conducted in biosafety level 3 animal facilities. BALB/c mice were imported from Harlan UK Ltd, UK, and those of 6-8 weeks of age were obtained from the Laboratory Animal Unit of the University of Hong Kong. Mice were housed at temperatures between 22 to 25° C. with dark/light cycles and given access to standard pellet feed and water ad libitum. For prophylactic study, one day before virus inoculation, each mouse was administered with 100 μL of mAb at 1 mg/mL intraperitoneally. For therapeutic study, infected mice were administered with 100 μL of mAb at 1 mg/mL intraperitoneally at day 1 post viral challenge. Mice in the control groups were administered with either PBS or with a non-H7N9 mAb. On the day of virus infection, each mouse was inoculated with 10 LD50 (40 μL) of H7N9/AH1 virus through intranasal route. Virus inoculation was performed under ketamine (100 mg/kg) and xylazine (10 mg/kg) anesthesia. The mice were monitored for 14 days with disease severity score and body weight recorded daily. Disease severity were scored as follow: Score 0, apparently healthy; Score 1 (mild disease symptom) with ruffled fur but still active; Score 2 (medium disease symptom) with ruffled fur, reduced activity and no weight gain; Score 3 (severe disease symptoms) with ruffled fur, hunched posture, labored breathing and weight loss; Score 4 (moribund) being very inactive, showing difficulty moving around and accessing to food and water, and weight loss. The predefined humane endpoints were either a weight loss of ≥20% or a disease severity score of 4. Mice were euthanized if the humane endpoints were reached. Each study group included 5-15 randomly allocated female mice for calculating survival rates and statistically significant differences based on the viral challenge model previously established with only female mice (see To K K, et al. Recombinant influenza A virus hemagglutinin HA2 subunit protects mice against influenza A (H7N9) virus infection. Archives of virology Arch Virol 160, 777-786 (2015); Zhao H, et al. A novel peptide with potent and broad-spectrum antiviral activities against multiple respiratory viruses. Sci Rep 6:22008 (2016); Zhao H, et al. Novel residues in the PA protein of avian influenza H7N7 virus affect virulence in mammalian hosts. Virology 498:1-8 (2016), each of which is incorporated by reference herein in its entirety). The investigators were blinded to animal group allocation during data collection.

### Example 15: Statistical Analysis

GraphPad Prism 9.5.1 was used to plot the ELISA data using sigmoidal dose-response with variable slope for curve fitting and neutralization data using 5-parameter nonlinear regression for curve fitting. All quantitative data are presented as mean±standard error (SEM). GraphPad Prism 9.5.1 was also used to plot the mouse survival curves. Two-sided unpaired student's t-test in GraphPad Prism 9.5.1 and Microsoft 365 Excel version 2404 was used for comparisons between mouse groups, and a P value of less than 0.05 was considered statistically significant. P values of 0.05-0.10 were considered statistical trends.

### Example 16: Isolation and Characterization of Human H7N9 mAbs In Vitro and Comparison of H7.HK2 to Previous H7 HA1-Directed Neutralizing mAbs

From a H7N9 convalescent case, four human monoclonal antibodies (mAbs)-namely, H7.HK1, H7.HK2, H7.HK3, and H7.HK4 that specifically bind to the hemagglutinin (HA) of the H7N9 influenza virus were isolated. These mAbs, defined by specific nucleic acid and amino acid sequences encoding the mAb variable regions, were isolated from individual B cells that specifically bound to the H7 HA. PBMCs were obtained from the 2013 H7N9 convalescent case in Hong Kong (H7N9_HK2013) with virus isolated as A/Hong Kong/470129/2013 H7N9 (K. K. To, et al., Unique reassortant of influenza A (H7N9) virus associated with severe disease emerging in Hong Kong. The Journal of infection 69, 60-68 (2014), incorporated by reference herein in its entirety). The course of infection lasted for about one month and the patient treatment required extracorporeal membrane oxygenation (ECMO) and i.v. zanamivir (K. K. To, et al., Unique reassortant of influenza A (H7N9) virus associated with severe disease emerging in Hong Kong. The Journal of infection 69, 60-68 (2014), incorporated by reference herein in its entirety).

On Nov. 17, 2013, a 36-year-old female was exposed to live poultry. On Nov. 23, 2013, the patient presented with a fever, cough, sputum, and sore throat. On Nov. 27, 2013, the patient was hospitalized with a fever of 40° C. A chest X-ray demonstrated right lower zone consolidation and right pleural effusion. On Nov. 29, 2013, the patient was intubated and started oseltamivir. On Nov. 30, 2013, the patient received ECMO. On Dec. 2, 2013, a H7N9 infection was confirmed via RT-PCR of an endotracheal aspirate sample. All 8 gene segments were sequenced, and the virus was isolated as A/Hong Kong/470129/2013. The patient was administered zanamivir i.v. On Dec. 6, 2013, the ECMO was stopped, and the patient recovered on Dec. 17, 2013. During the hospital stay, there were indicators for deranged liver and kidney functions, but these indicators returned to normal.

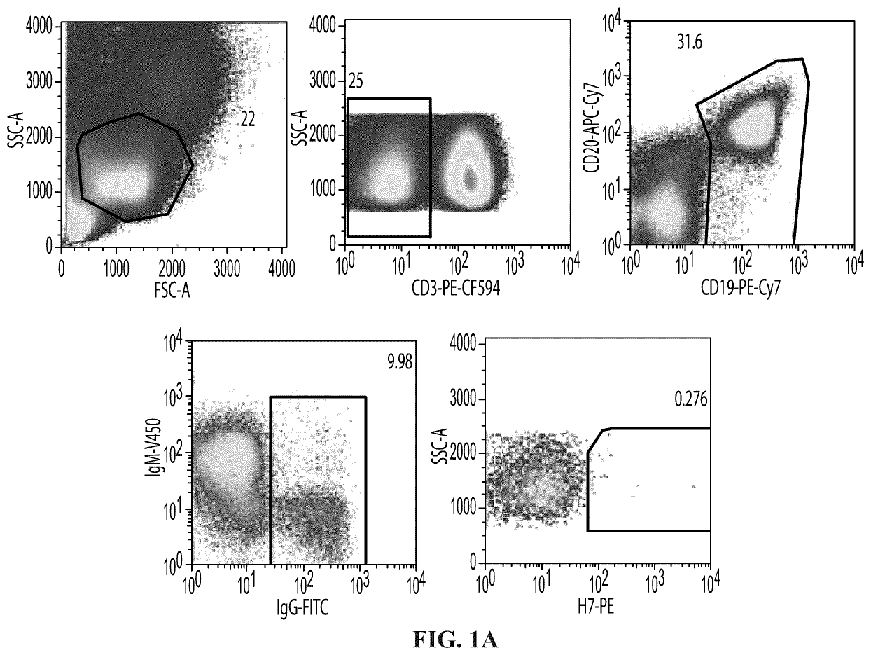

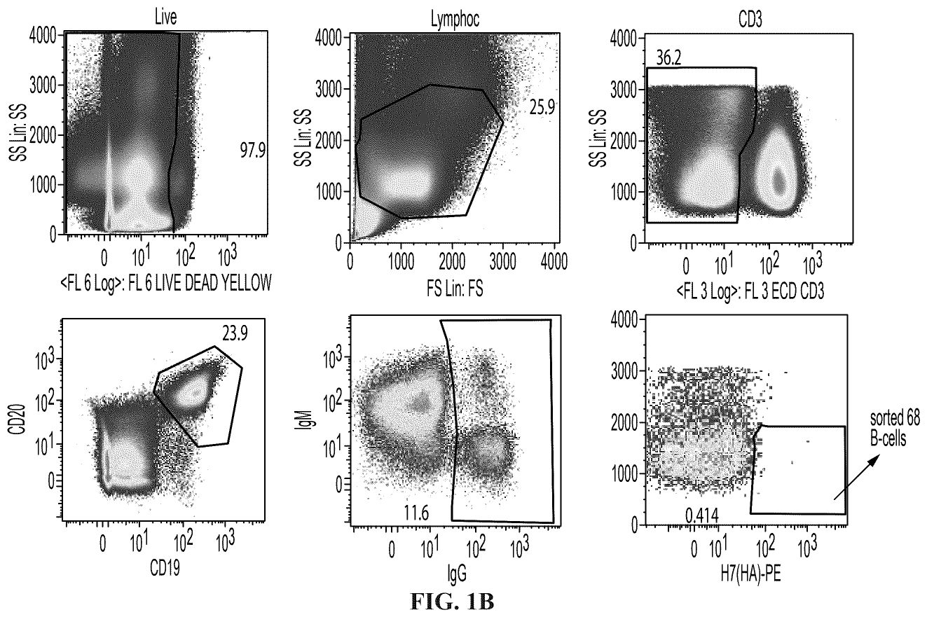

The PBMC sample used to isolate mAbs was collected one year post recovery. The methods included sorting HA-specific individual B-cells from a PBMC sample of the HK_H7N9 case, RT-PCR amplifying the heavy and light chain variable regions, cloning and expression paired heavy and light chain variable regions and purifying and characterizing the expressed mAbs. For H7-specific mAb isolation, a soluble recombinant H7 HA antigen based on A/Shanghai2/2013 H7N9 was used for biotinylation, followed by streptavidin-PE conjugation. FIG. 1A-B show FACS depicting the staining and selection for H7N9 HA-specific B cells from the H7N9.HK donor PBMCs 1 year post recovery (SSC-A, side scatter area; FSC-A, forward scatter area). As shown in FIG. 1A-B, this H7-PE bait was used to stain 5 million PBMCs from the H7N9.HK2013 donor and to sort a total of 68 IgG+ B cells (defined as CD3-CD19+CD20+IgG+) that are H7-PE+. Most of the sorted cells were at the borderline for H7-PE staining, but a few stained brightly for H7-PE.

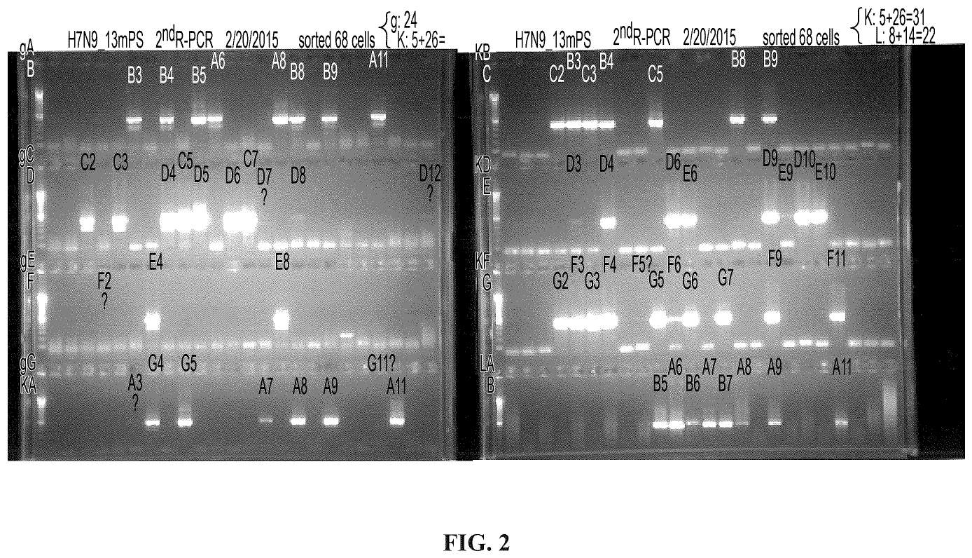

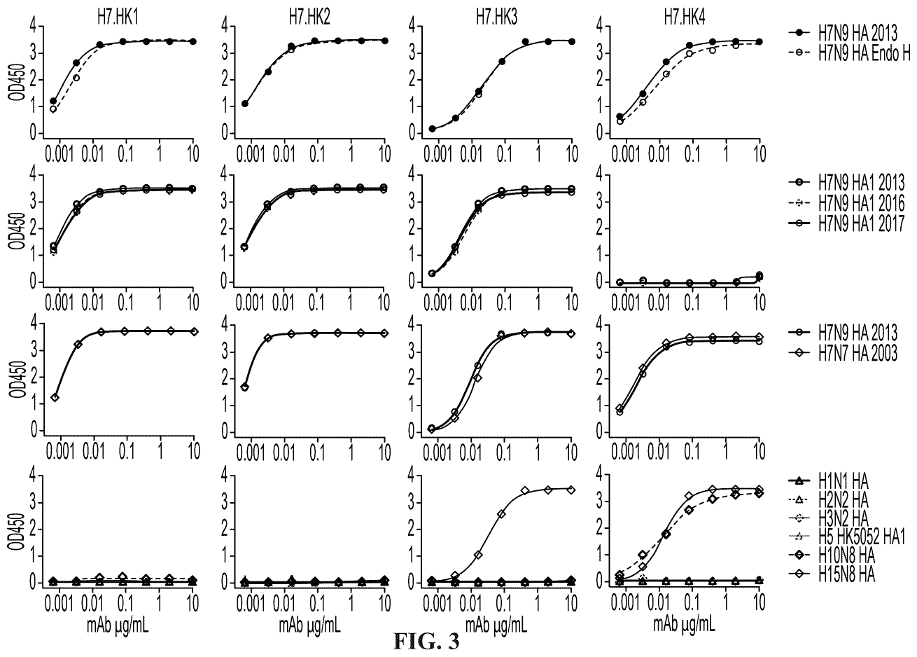

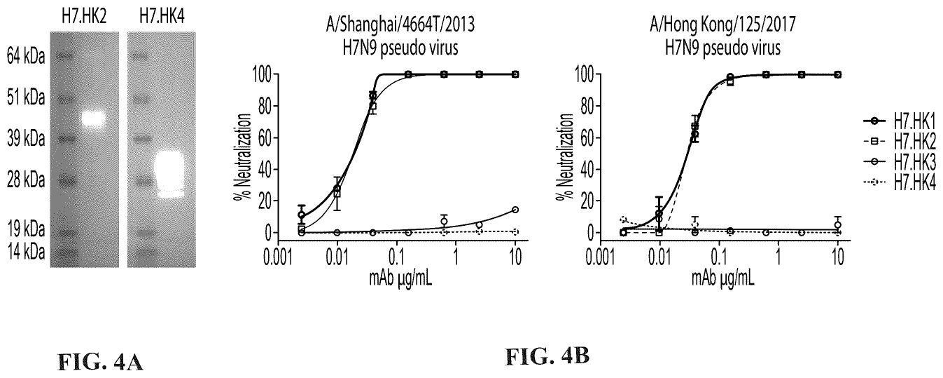

From the sorted B cells, single B cell RT-PCR was performed and four H7-specific mAbs were recovered: H7.HK1, H7.HK2, H7.HK3, and H7.HK4. FIG. 2 shows single B-cell RT-PCR results. The left DNA gel shows PCR amplicons of IgG heavy chain variable region. The right DNA gel shows PCR amplicons of kappa chain variable region. FIG. 3 shows ELISA binding curves of the indicated mAbs to soluble recombinant H7N9 HA and H7N7 HA, with or without Endo H treatment (upper panels), to the matching H7N9 HA1 from 2013, HA1s from 2016 and 2017, H7N9 HA from 2013, and H7N7 HA from 2003 (middle panels), and to 6 other non-H7 HA or HA1 proteins (lower panels). As shown in FIG. 3 (upper panels), ELISA results revealed that these mAbs bound tightly to the H7N9 HA recombinant antigen used for H7-PE and to a recombinant H7N7 HA antigen based on strain A/Netherlands/219/2003 H7N7. FIG. 3 (middle panels) shows that after switching the ELISA coating antigen to only the globular head domain HA1 of the matching H7N9 HA, the binding curves of H7.HK1, H7.HK2, and H7.HK3 (for 2013, 2016, and 2017) were fully maintained, indicating that these mAbs bind to the globular head domain HA1; meanwhile, H7.HK4 lost binding to H7N9 HA1, indicating that its binding epitope is likely located in the stalk region HA2. As shown in FIG. 3, upper and middle panels, pre-treating the H7N9 HA or HA1 with Endoglycosidase H (Endo H) had no effect on the mAb binding profiles, indicating that these mAbs do not rely on glycans to bind the H7 antigen. To assess cross-reactivity, these mAbs were tested for binding to 6 other non-H7 HA or HA1 recombinant proteins. As shown in FIG. 3, lower panels, H7.HK1 and H7.HK2 did not react with any non-H7 HA or HA1 tested, while H7.HK3 cross-reacted with H15N8 HA, and H7.HK4 cross-reacted with H10N8 and H15N8 HAs, which sequence-wise are the closest to H7 in influenza group 2 HAs (S. J. Gamblin, & J. J. Skehel, Influenza hemagglutinin and neuraminidase membrane glycoproteins. J Biol Chem 285, 28403-28409 (2010), incorporated by reference herein in its entirety). FIG. 4A shows a Western blot of a cleaved HA based on A/Shanghai/1/2013 H7N9 which confirmed that H7.HK2 binds to HA1 and H7.HK4 binds to HA2.

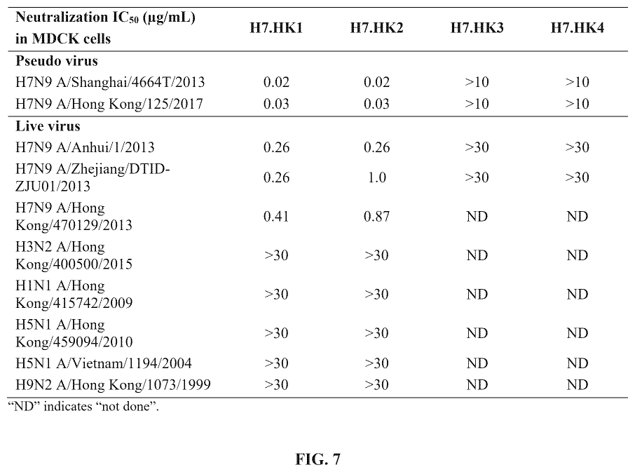

Two of the mAbs-namely, H7.HK1 and H7.HK2, demonstrated potent allosterically neutralizing activity against H7N9 infection in vitro. FIG. 4B and FIG. 7 shows neutralization IC50s of the H7.HK mAbs against the indicated H7N9 pseudo virus or other live replicating influenza isolates in Madin-Darby Canine Kidney (MDCK) cells (“ND” indicates “not done”). As shown in FIG. 4B and FIG. 7, the H7N9 pseudo virus particles were generated and the mAb neutralizing activity was tested by a luciferase readout from single round infection of the pseudo virus in MDCK cells using expression plasmids separately encoding H7 and N9 based on H7N9 strain A/Shanghai/4664T/2013 H7N9 to pseudo virus with HIV-1 NL4-3Aenv.Luc backbone (C. Qiu, Y. et al., Safe pseudo virus-based assay for neutralization antibodies against influenza A(H7N9) virus. Emerging infectious diseases 19, 1685-1687 (2013), incorporated by reference herein in its entirety). H7.HK1 and H7.HK2 each potently neutralized the H7N9 pseudo virus with an IC₅₀ of 20 ng/ml, while the other two mAbs, H7.HK3 and H7.HK4, did not neutralize the pseudo virus at up to 10 μg/mL (FIG. 4B and FIG. 7, left). Similarly, pseudo virus was generated using the H7 from A/Hong Kong/125/2017 H7N9. Both H7.HK1 and H7.HK2 retained their neutralization titers against the H7N9 2017 pseudo virus with an IC₅₀ of 30 ng/mL, while the other two mAbs H7.HK3 and H7.HK4 did not neutralize (FIGS. 4B and 7). As shown in FIG. 7, the mAb neutralizing activity was further assessed against three live replicating H7N9 viruses, A/Anhui/1/2013, A/Zhejiang/DTID-ZJU01/2013, and the donor's autologous isolate A/Hong Kong/470129/2013, for multiple rounds of infection in MDCK cells. Scored by the presence of cytopathic effect (CPE), mAbs H7.HK1 and H7.HK2 neutralized all three H7N9 isolates with IC50s ranging 0.26-1.0 μg/mL; however, they did not neutralize any non-H7N9 influenza isolates tested, indicating that these mAbs are specific to H7N9 strains (FIG. 7). The other two mAbs H7.HK3 and H7.HK4 did not neutralize any of the tested H7N9 viruses and therefore were not tested against non-H7N9 viruses.

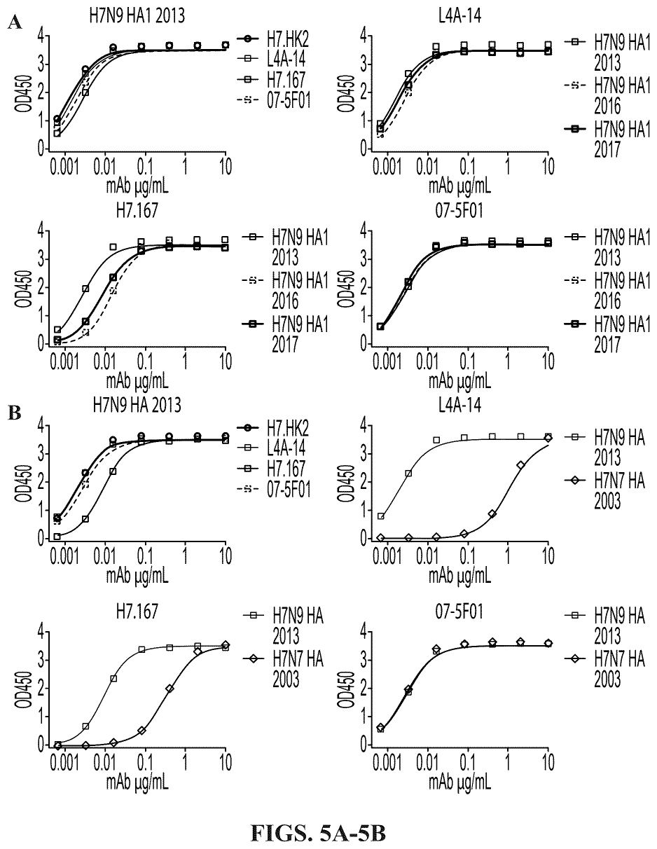

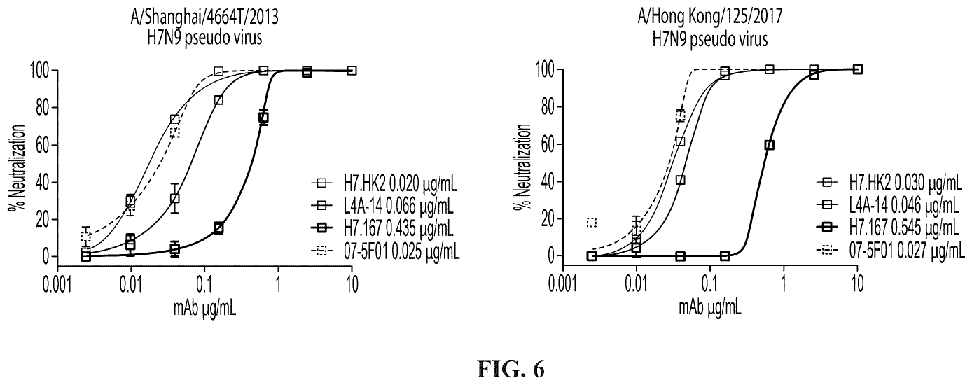

Three previous mAbs representing the best from each corresponding study were compared to H7.HK2 for ELISA binding to H7 antigens and neutralization of H7N9 pseudo viruses. Cloned from plasmablasts of an acute infection in 2013-2014 (Huang K A, et al, Structure-function analysis of neutralizing antibodies to H7N9 influenza from naturally infected humans. Nature Microbiology, which is hereby incorporated by reference in its entirety.) mAb L4A-14 is directed at the receptor-binding site (RBS) and bound 2013 H7N9 HA1 and HA similarly to H7.HK2, retained full binding to 2016 and 2017 HA1s (FIG. 5A) but lost vast majority of binding to H7N7 HA (FIG. 5B). Derived from EBV transformed B cells after vaccination, another RBS-directed mAb H7.167 bound less well than H7.HK2 to 2013 H7N9 HA1 and HA and lost substantial binding to 2016 and 2017 HA1s and to H7N7 HA (see Thornburg N J, et al. H7N9 influenza virus neutralizing antibodies that possess few somatic mutations. J Clin Invest 126:1482-94 (2016), incorporated by reference herein in its entirety). Cloned from plasmablasts after vaccination (see Henry Dunand C J, et al. Both Neutralizing and Non-Neutralizing Human H7N9 Influenza Vaccine-Induced Monoclonal Antibodies Confer Protection. Cell Host Microbe 19:800-13 (2016), incorporated by reference herein in its entirety), the non-RBS mAb 07-5F01 bound 2013, 2016, and 2017 H7N9 HA1s similarly to H7.HK2 and fully retained reactivity to H7N7 HA (see Henry Dunand C J, Leon P E, Huang M, Choi A, Chromikova V, Ho I Y, Tan G S, Cruz J, Hirsh A, Zheng N Y, Mullarkey C E, Ennis F A, Terajima M, Treanor J J, Topham D J, Subbarao K, Palese P, Krammer F, Wilson P C. 2016. Both Neutralizing and Non-Neutralizing Human H7N9 Influenza Vaccine-Induced Monoclonal Antibodies Confer Protection. Cell Host Microbe 19:800-13, which is hereby incorporated by reference in its entirety). FIG. 6 shows pseudo virus neutralization data which indicates the RBS-directed mAbs H7.167 and L4A-14 neutralized the 2013 H7N9 weaker than H7.HK2. L4A-14 neutralized the 2017 strain slightly better than 2013 H7N9, but H7.167's activity was further reduced by the 2017 virus. The non-RBS mAb 07-5F01 neutralized both 2013 and 2017 H7N9 viruses with comparable potency to H7.HK2. Hence, evaluated by H7 antigen binding and pseudo virus neutralization, H7.HK2 is superior to the two best previous RBS-directed mAbs L4A-14 and H7.167 and matches the one best previous non-RBS mAb 07-5F01 against H7N9.

FIG. 6 shows neutralization curves of H7.HK mAbs against the H7N9 pseudo virus in MDCK cells. Data are shown as mean±SEM. The neutralization IC₅₀s of H7.HK1 and H7.HK2 using the pseudo virus were about 10-fold more potent than those using the live replicating viruses, suggesting that the more sensitive pseudo virus neutralization assay is useful for the initial screening of neutralizing mAbs, which could then be confirmed with live replicating viruses. Similar differences in IC₅₀ values have also been reported for other HA-specific mAbs previously tested by both pseudo viruses and live replicating viruses (D. Corti, et al., A neutralizing antibody selected from plasma cells that binds to group 1 and group 2 influenza A hemagglutinins. Science 333, 850-856 (2011), incorporated by reference herein in its entirety). FIG. 8 shows neutralization of the H7.HK mAbs against H7N9/AH1 in MDCK cells. In FIG. 8 the neutralization was assessed with live replicating viruses; “+” indicates infectivity identified by the presence of cytopathic effect (CPE). Columns represent 5 replicates.

### Example 17: Sequence Analysis of the Isolated Human H7N9 mAbs

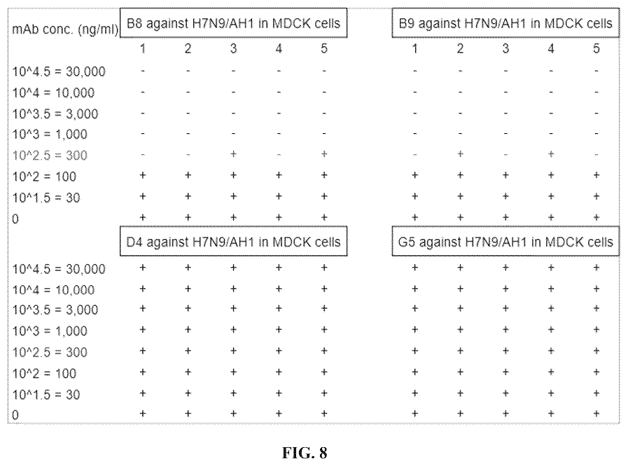

FIGS. 9 and 10 show a summary of the genetic composition, epitope, and neutralization function of the isolated H7N9 mAbs. As shown in FIG. 9, sequence analysis revealed that all four H7.HK mAbs are IgG1. FIG. 9 also shows H7.HK1 and H7.HK2 are IgG1 clonal variants using IGHV4-59 for heavy chain with 8-10% somatic hypermutation (SHM) and a CDR3 of 11 amino acids according to the Kabat and Chothia definition (C. Chothia, & A. M. Lesk, Canonical structures for the hypervariable regions of immunoglobulins. J Mol Biol 196, 901-917 (1987); C. Chothia, A. et al., Conformations of immunoglobulin hypervariable regions. Nature 342, 877-883 (1989); and B. Al-Lazikani, et al., Standard conformations for the canonical structures of immunoglobulins. J Mol Biol 273, 927-948 (1997), each of which are incorporated by reference herein in its entirety), and IGKV2-28 for light chain with 6% SHM and a CDR3 of 9 amino acids. FIG. 10 shows protein sequences of the heavy and light chain variable region of H7N9 mAbs aligned to the putative germline V-genes, with amino acid substitutions highlighted in red, and in magenta for substitutions shared between the clonally related mAbs H7.HK1 and H7.HK2. Spaces are added to maintain alignments; framework regions (FR) and complementarity-determining regions (CDRs) are indicated based on the Chothia nomenclature. Highlighted in yellow are the mAb residues (paratopes of H7.HK1 and H7.HK2) contacting the H7 antigen. The N-linked glycosylation sites on the light chain CDR1s of H7.HK1 and H7.HK2 are underlined. FIG. 10 shows that H7.HK1 and H7.HK2 sequences are different from each other, sharing only 3 out of 13-15 amino acid SHMs in the heavy chain V-gene region and only 1 out of 8 amino acid SHMs in the light chain V-gene region. H7.HK3 uses IGHV7-4-1 for heavy chain with 7% SHM and a CDR3 of 14 amino acids, and IGKV1-5 for light chain with 5% SHM and a CDR3 of 8 amino acids. H7.HK4 uses IGHV4-61 for heavy chain with 7% SHM and a CDR3 of 13 amino acids, and IGKV1-16 for light chain with 5% SHM and a CDR3 of 9 amino acids (FIG. 9).

### Example 18: Structural Analysis of H7.HK1 and H7.HK2 Demonstrates an Unique Mode of Neutralization

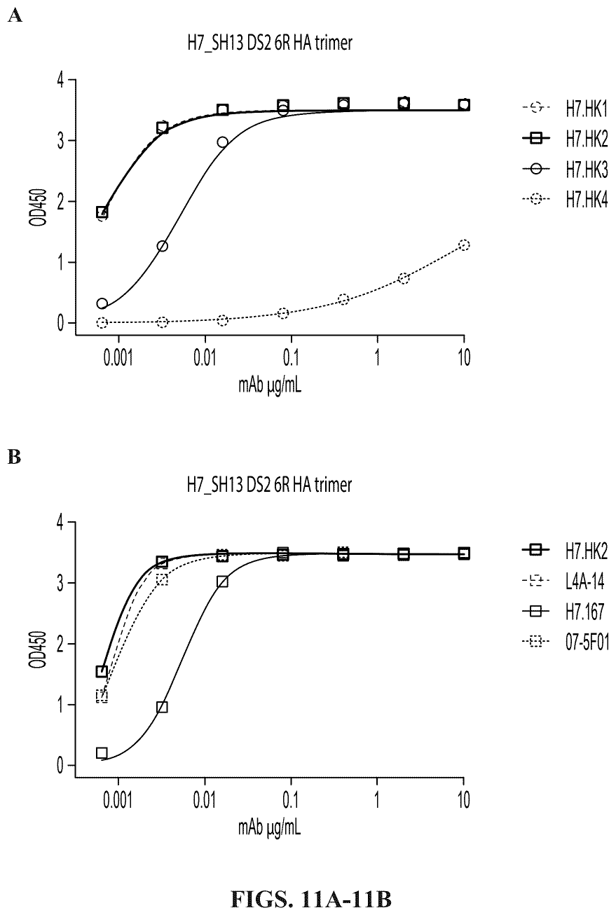

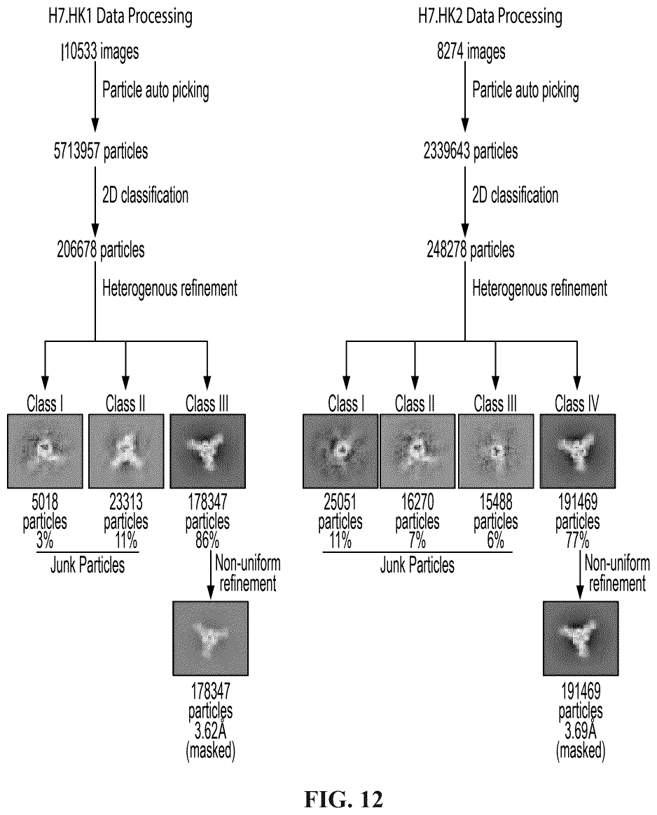

Cryo-EM structures of the antigen-binding fragments (Fabs) complexed with H7 HA revealed that H7.HK1 and H7.HK2 bind to the β14-centered surface of H7 HA1, partially overlapping with the antigenic site D of the globular head of HA and disrupt the 220-loop that makes hydrophobic contact with sialic acid on the adjacent protomer, thus effectively blocking the viral entry. For structural analysis, the antibody Fabs were generated and the H7 HA trimer was expressed by transient transfection of Expi293F cells. A soluble, disulfide-stabilized, and fully cleaved H7 HA trimer was expressed via transient transfection of Expi293F cells. FIG. 11A shows that H7.HK1 and H7.HK2 bound the H7 HA trimer tightly, H7.HK3 bound less well, and H7.HK4 did not bind at all. The three previous neutralizing mAbs all bound the H7 HA trimer tightly, with H7.167 showing weaker binding (FIG. 11B). The antibody fragments were generated for antigen binding (Fabs) of H7.HK1 and H7.HK2 to bind the H7 HA trimer. As shown in FIG. 12, the grids containing Fab: HA complexes were frozen and cryo-EM structures of each Fab bound to each H7 HA trimer were determined (FIG. 12).

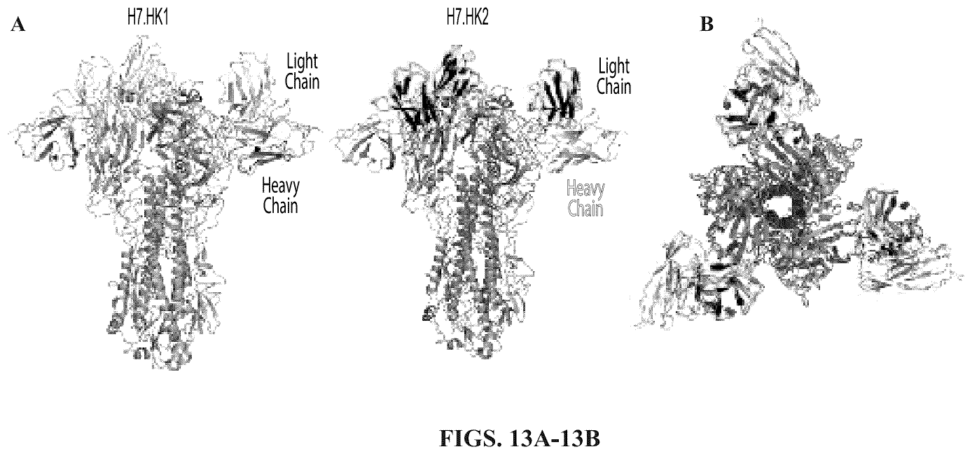

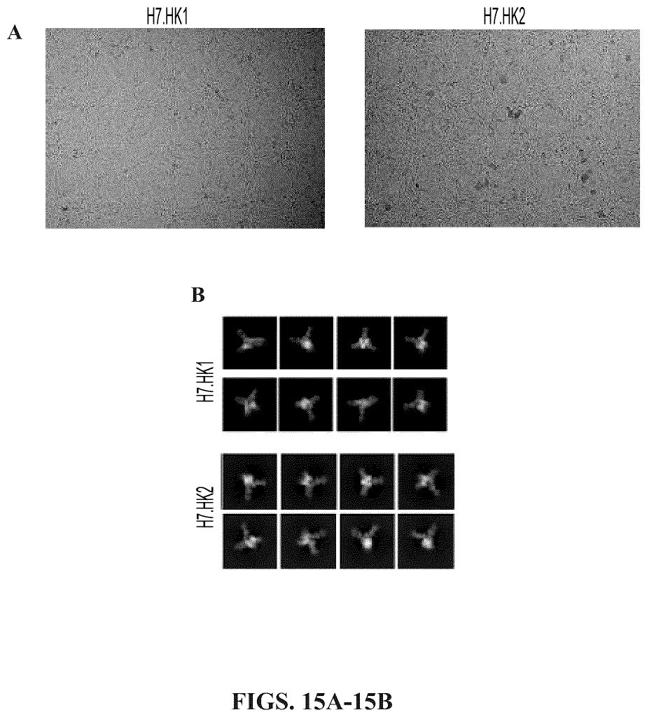

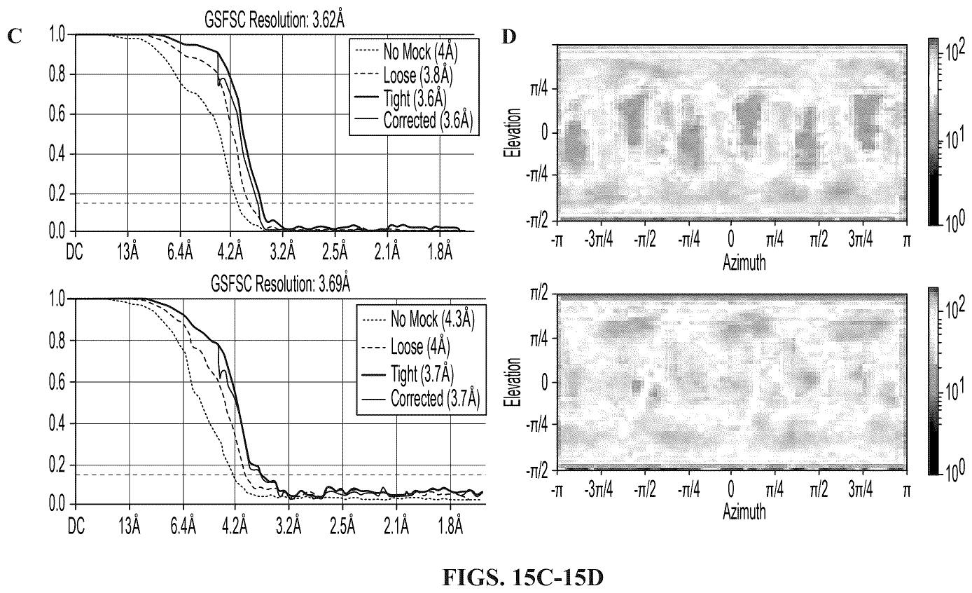

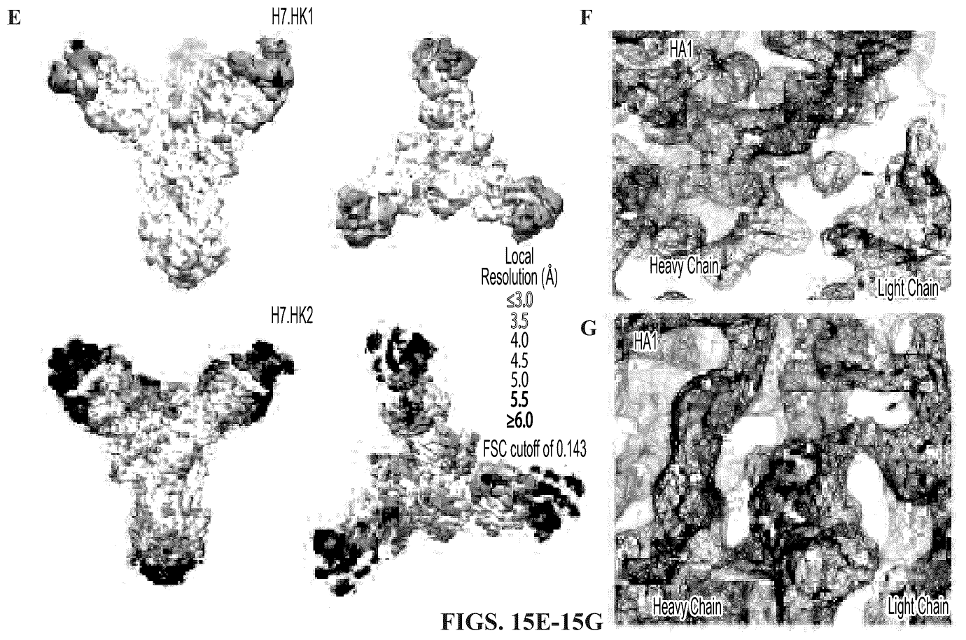

FIGS. 13-15 show cryo-EM details of H7.HK1 and H7.HK2 in complex with H7 SH13 DS2 6R HA trimer. FIG. 15A depicts representative micrograph of H7.HK1 (left) and H7.HK2 (right). FIG. 15B depicts representative 2D class averages of H7.HK1 and H7.HK2 and demonstrates that H7.HK1 and H7.HK2 are highly superimposable. FIG. 15C shows that the gold-standard Fourier Shell Correlation (FSC) resulted in a resolution of 3.62 Å for the overall map of H7.HK1 and 3.69 Å for the overall map of H7.HK2. Non-uniform refinement with C3 symmetry was used for both reconstructions. FIG. 15D depicts a heatmap showing the orientations of all particles used in the final refinement. FIG. 15E depicts the local resolution of the final overall map, contoured at 0.0989 for both structures. Resolution estimation was generated through cryoSPARC using an FSC cutoff of 0.5. FIG. 15F depicts representative density for the interface of H7.HK1 heavy chain, light chain, and H7 HA. FIG. 15G depicts representative density for the interface of H7.HK2 heavy chain, light chain, and H7 HA.

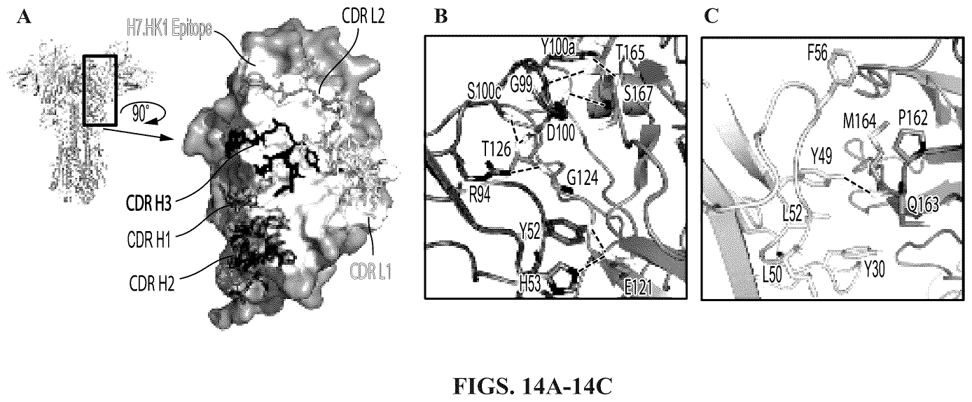

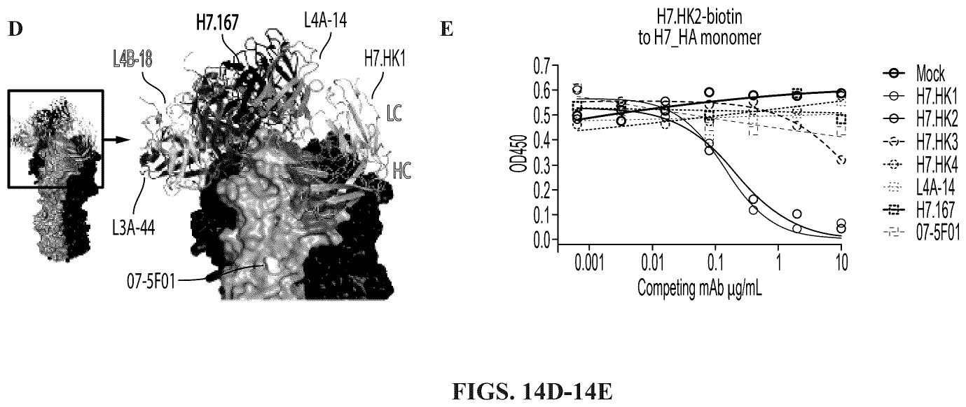

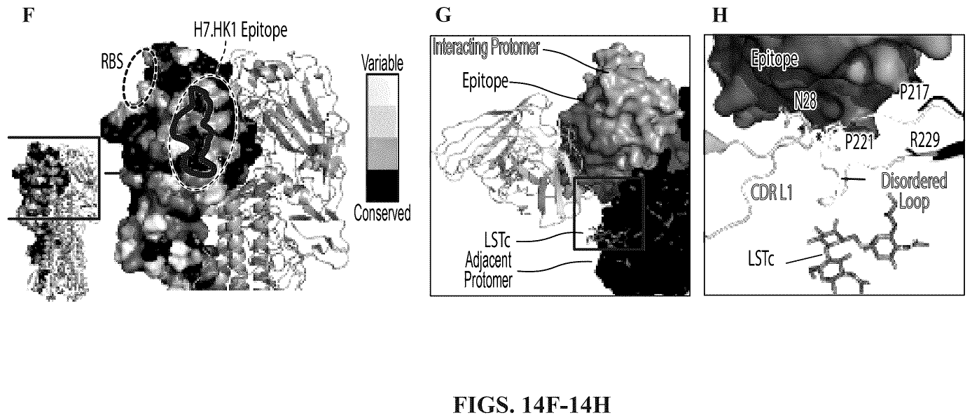

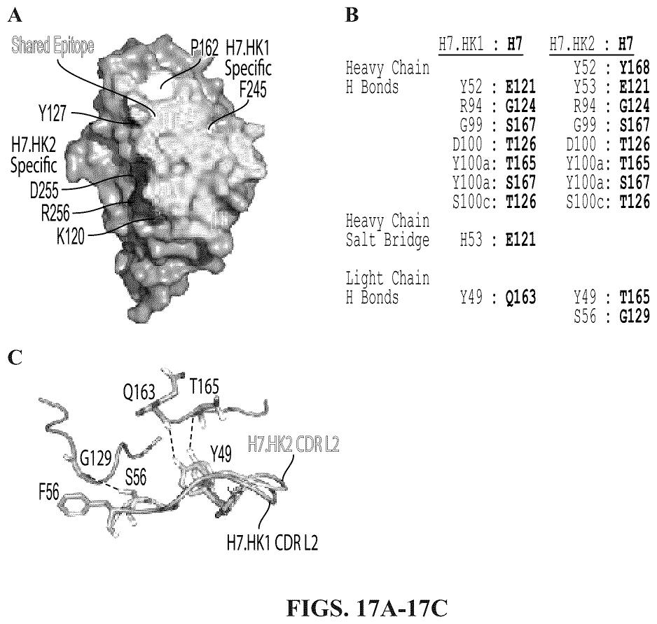

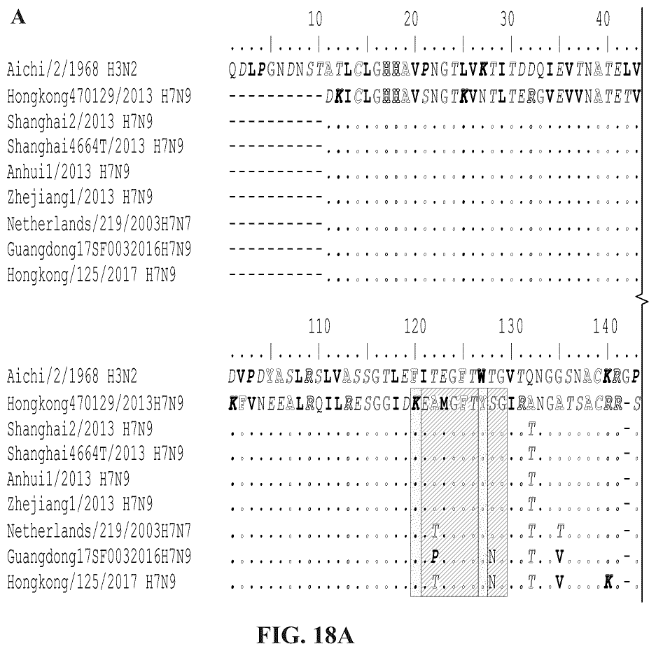

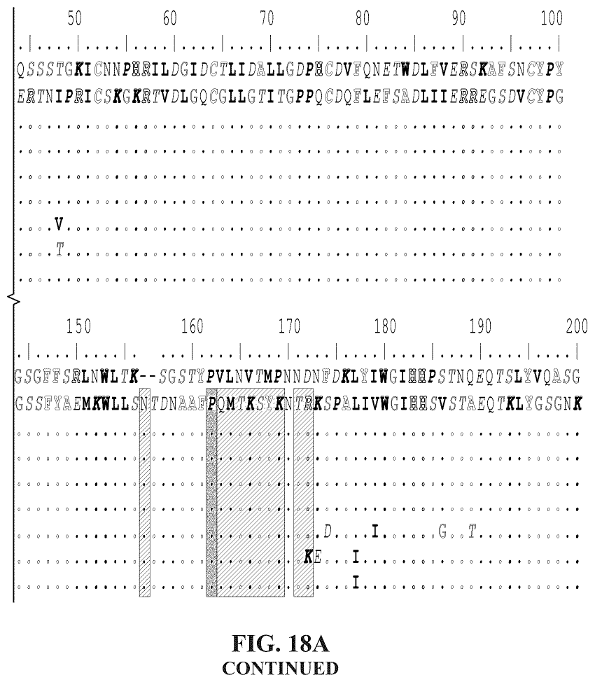

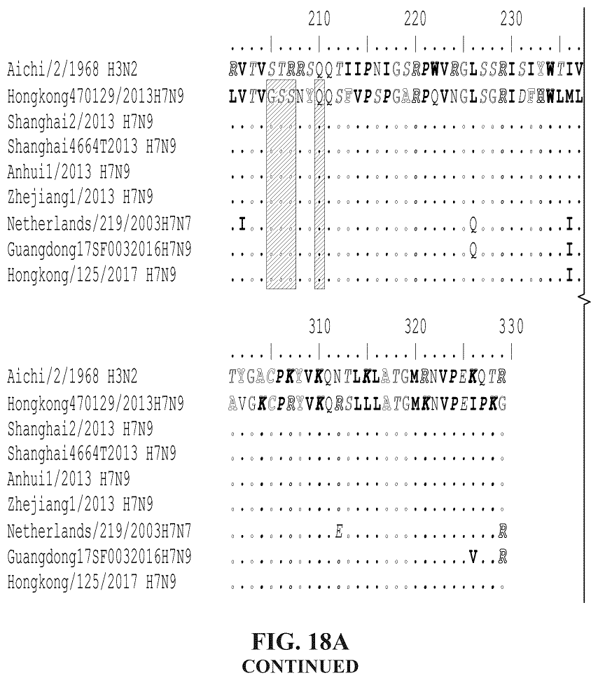

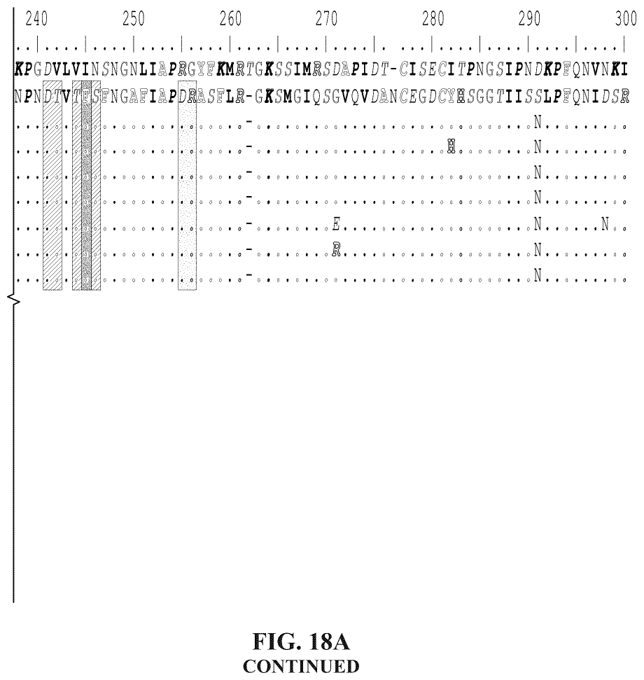

FIGS. 13A-B, 14A-G, and FIGS. 15A-D show additional structural analysis of H7.HK1 and H7.HK2 in complex with H7 HA trimer. FIG. 13A shows cryo-EM structures of H7.HK1 and H7.HK2 bound to H7 in the head region. As shown in FIG. 13A, a resolution of 3.62 Å for H7.HK1 and 3.69 Å for H7.HK2 was achieved. FIG. 13B shows a top view of alignment of H7.HK land H7.HK2 structures. As shown in FIGS. 13B and 14A, these complex structures demonstrate that H7.HK1 and H7.HK2 mAbs are completely superimposable (FIG. 13A) and their interactions with H7 are centered at β14 and extended to the surfaces of β10 and −19 (FIG. 14A). This β14-targeting surface partially overlaps with antigenic site D towards the A and B sites (see also FIG. 14A). FIG. 14A shows surface presentation of the H7.HK1 epitope (orange) on H7 HA1, with interacting CDRs. As shown in FIGS. 17A-C, analysis of the binding epitope demonstrates that most interactions are driven by the heavy chain and consist of seven hydrogen bonds (Y52: E121, R94: G124, G99: S167, D100: T126, Y100a: T165, Y100a: S167, S100c: T126) and one salt bridge (H53: E121) (FIG. 15B). FIG. 14C shows that the light chain is less involved in binding, making only one hydrogen bond (Y49: Q163) and weak hydrophobic interactions, and the interactions are stabilized by hydrophobic residues on the periphery of the light chain interface. The light chain of both H7.HK1 and H7.HK2 are glycosylated in CDR L1; this glycan plays no role in binding, but there is good density to support its presence. FIG. 17A shows that the epitope of H7.HK2 is similar to that of H7.HK1, only differing in slight contacts on the periphery. FIG. 17B indicates nearly all hydrogen bonds are conserved between the two. However, the substitution of F56S in CDR L2 of H7.HK2 results in an additional hydrogen bond with HA G129 (FIG. 17C). FIG. 17C shows that this substitution also shifts the orientation of H7.HK2 CDR L2 slightly so that Y49 interacts with T165 for H7.HK2 instead of Q163 for H7.HK1. As H53 is substituted with tyrosine in the heavy chain of H7.HK2, it does not make the H53: E121 salt bridge.

FIG. 14D shows the comparison of the binding site of H7.HK1 to that of four other H7-reactive antibodies with published structures, L4A-14, L4B-18, L3A-44 (PDB: 6II4, 6II8, 6I19; K. A. Huang, et al., Structure-function analysis of neutralizing antibodies to H7N9 influenza from naturally infected humans. Nat Microbiol 4, 306-315 (2019), incorporated by reference herein in its entirety), and H7.167 (PDB: 5V2A; N. J. Thornburg, et al., H7N9 influenza virus neutralizing antibodies that possess few somatic mutations. The Journal of clinical investigation 126, 1482-1494 (2016), incorporated by reference herein in its entirety). The binding site of H7.HK1 is also distant from that of 07-5F01 (PMID: 27281570), which was mapped to an escape mutation R65K (corresponding to R57K here by H3 numbering) of HA1. This analysis demonstrates that the binding site of H7.HK1 is almost completely distinct from that of these previously published antibodies, which compete for the receptor binding site (RBD) (FIG. 14D). Direct competition ELISA applying biotin labeled H7.HK2 to bind H7 HA did not detect any effective competition by H7.HK3, H7.HK4, L4A-14, H7.167, and 07-5F01 (FIG. 14D), confirming the unique location of H7.HK1 and H7.HK2 epitopes. Further analysis of N=1,483 H7 HA1 amino acid sequences from the Global Initiative on Sharing All Influenza Data (GISAID) revealed a conserved lateral patch (FIG. 14F). Strikingly, the lateral patch epitope of H7 (β14-centered) was distal to the receptor binding site of the protomer with which it interacted and is closer to the receptor binding site on the adjacent protomer.

FIG. 14G shows modeling of the binding site of human receptor analogue Sialylneolacto-N-tetraose c (LSTc) (red) based on a previous crystal structure (PDB: 4BSE) onto H7 from the H7.HK1 complex, demonstrating that H7.HK1 does not compete with sialic acid on the adjacent protomer. To analyze the mechanism of neutralization, the human receptor analogue Sialylneolacto-N-tetraose c (LSTc) was modeled into the receptor binding site of H7 in the H7.HK1 complex. As shown in FIG. 14G, interestingly, there were no steric clashes between H7.HK1 and sialic acid on the adjacent promoter, and no mAb interaction with the sialic acid binding site. FIGS. 14G and 14H show alignment of H7.HK1 complex with a previous crystal structure of H7 (PDB: 4BSE) and shows that the 220-loop (G218-G228) required for sialic acid binding is disordered in the complex structure and would clash with the H7.HK1 light chain. However, as shown in FIG. 14H, the 220-loop that makes hydrophobic contact with sialic acid presents no density in the structure of H7.HK1 or H7.HK2 bound to HA, suggesting that the antibody binding causes this loop to become disordered. All previously examined H7 structures (FIG. 14D), as well as an additional cryo-EM structure in which Fab 1D12 is bound to the stem region of H7 HA (PDB: 6WXL) (3) have consistent electron density accounting for this loop. Alignments of the H7.HK1 complex structure with the crystal structure of H7 HA bound to LSTc demonstrate where this loop would be and that the light chain of H7.HK1 would clash with this loop, suggesting that the binding of H7.HK1 and H7.HK2 cause this 220-loop to become disordered and thus preventing its binding to sialic acid. The HA1 trimer interface mAb FluA-20 interacts with the non-RBS side of 220-loop on the protomer it interacts with (see Bangaru S, Lang S, Schotsaert M, Vanderven H A, Zhu X, Kose N, Bombardi R, Finn J A, Kent S J, Gilchuk P, Gilchuk I, Turner H L, Garcia-Sastre A, Li S, Ward A B, Wilson I A, Crowe J E, Jr. 2019. A Site of Vulnerability on the Influenza Virus Hemagglutinin Head Domain Trimer Interface. Cell 177:1136-1152 e18, which is incorporated by reference herein in its entirety). This mechanism of neutralization is distinct from those reported previously, which all directly compete with sialic acid for binding to HA on the protomer they interact with (K. A. Huang, P. et al., Structure-function analysis of neutralizing antibodies to H7N9 influenza from naturally infected humans. Nat Microbiol 4, 306-315 (2019); N. J. Thornburg, et al., H7N9 influenza virus neutralizing antibodies that possess few somatic mutations. The Journal of clinical investigation 126, 1482-1494 (2016); and J. Wang, et al., Characterization of Two Human Monoclonal Antibodies Neutralizing Influenza A H7N9 Viruses. Journal of virology 89, 9115-9118 (2015), each of which is incorporated by reference herein in its entirety).

FIGS. 17A-17C shows a comparison of H7.HK1 and H7.HK2 binding to H7. FIG. 17A depicts the difference in epitope of H7.HK1 and H7.HK2, showing that the majority of surface contacts are conserved (shown in orange). H7.HK1 specific surfaces are shown in magenta, H7.HK2 specific surfaces are shown in cyan. The light chain of both H7.HK1 and H7.HK2 are glycosylated in the CDR L1 region; this glycan plays no role in binding, but there is good density to support its presence. As shown in FIG. 17A, the epitope of H7.HK2 is similar to that of H7.HK1, only differing in slight contacts made with residues on the periphery. FIG. 17B shows hydrogen bonds and salt bridges formed by H7.HK1 and H7.HK2 with H7. Additionally, as shown in FIG. 17B, nearly all hydrogen bonds are conserved between the two antibodies. FIG. 17C depicts the differences in CDR L2 binding to H7 by H7.HK1 and H7.HK2 as a result of F56S substitution in H7.HK2. The substitution of F56S in CDR L2 of H7.HK2 forms an additional hydrogen bond with HA G129. As shown in FIG. 17C, the residue S56 also shifts the orientation of CDR L2 slightly so that Y49 interacts with T165 for H7.HK2 instead of Q163 for H7.HK1. Finally, as H53 is substituted with tyrosine in the heavy chain of H7.HK2, it does not make the H53: E121 salt bridge (FIG. 17B).

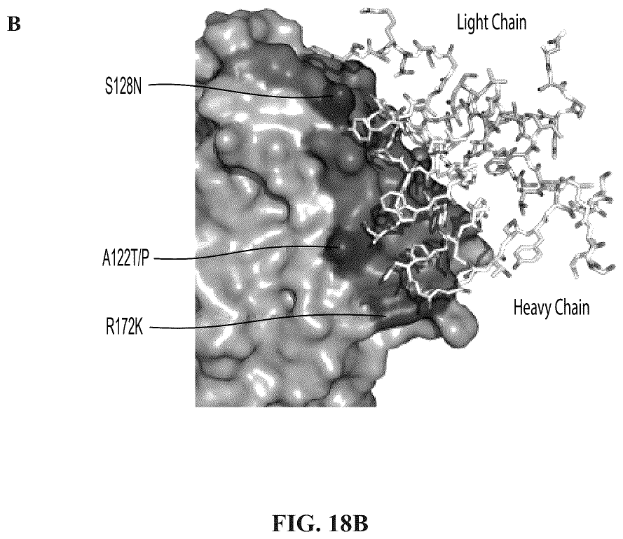

Since the H7N9 HA gene has significantly evolved and changed in 2016-2017 compared to that of 2013, the locations of mutated residues in the epitopes of H7.HK1 and H7.HK2 that consist of 32 contacting residues in HA1 were examined for both mAbs. FIGS. 18A-18B show antigenic drift of H7 HA1 in 2016-2017. FIG. 18A depicts H7 HA1 protein sequences from the indicated viral isolates aligned to the 2013 Hong Kong H7N9 autologous isolate at top, with identical amino acids shown in dots. Highlighted in yellow are the H7 residues (epitope) forming contacts with both mAbs H7.HK1 and H7.HK2. H7.HK1 specific epitopes are in magenta; H7.HK2 specific epitopes are in cyan. FIG. 18B depicts surface presentation of the H7 HA1 domain highlighting the epitopes (orange) of mAbs H7.HK1 and H7.HK2, with three mutations in red that appeared in the 2016-2017 viral isolates of H7N9. FIG. 18A and FIG. 18B show three mutations in the binding site of H7.HK1 and H7.HK2-namely, A122T/P, S128N, and R172K, appeared in 2016-2017 compared to the 2013 H7N9, and all three mutations are located at one side edge of the epitopes (see FIG. 18B). Any given H7 antigen can include one or more of the 3 mutations. For example, as shown in FIG. 18A, Guangdong 2016 H7 has all 3 mutations (A122P, S128N, and R172K), and Hongkong 2017 H7 has only 2 mutations (A122T and S128N). This analysis is in consistency with the intact binding profiles of H7.HK1 and H7.HK2 to both 2016 and 2017 HA1s aligned to the 2013 HA1 (see FIG. 3, middle panels) and H7.HK2's full retention of neutralization against the H7N9 2017 pseudo virus (FIG. 4B).

### Example 19: Prophylactic and Therapeutic Effectiveness of Human H7N9 mAbs in Mice Challenged with H7N9 Virus

Prophylaxis with passively administered mAb H7.HK1 or H7 HK2 conferred protection or benefit against H7N9 challenge in mice. The HA2-directed mAb H7.HK4 lacked neutralizing activity but protected mice from H7N9 challenge when engineered to mouse IgG2a with Fc effector function. Used in combination with H7.HK2 at a suboptimal dose, H7.HK4 augmented the mouse protection.

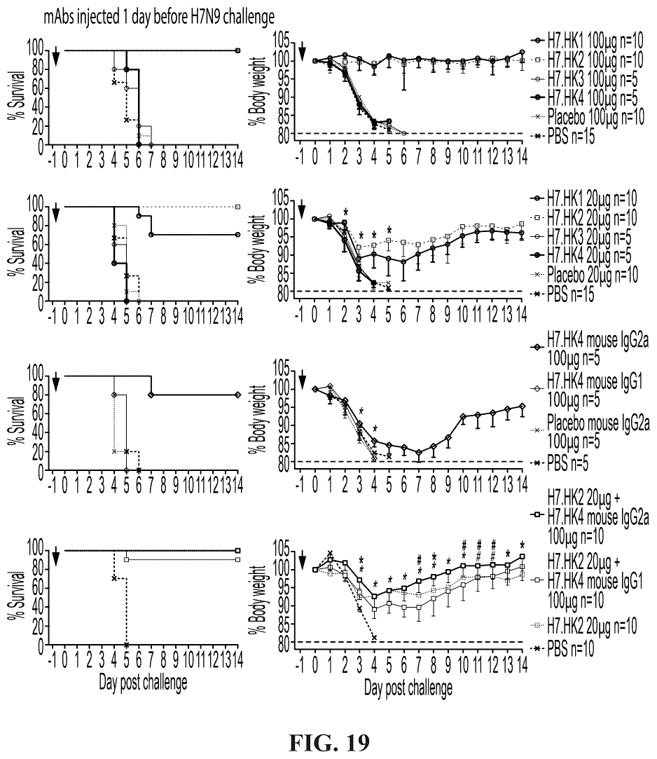

The prophylactic and therapeutic effectiveness of the isolated H7N9 mAbs was tested in a mouse lethal challenge model. To assess mAb prophylactic effect, BALB/c mice (n=5-10 per group from 1-2 experiments) were injected intraperitoneally (i.p.) with either 100 μg or 20 μg of human H7N9 mAbs one day before intranasal (i.n.) challenge of 10-fold 50% lethal dose (10 LD₅₀) of A/Anhui/1/2013 H7N9 virus. FIG. 19 shows the % survival (less than 20% weight loss) and % body weight of the survived mice plotted over time. Given 100 μg per mouse (equivalent to 5 mg/kg), the neutralizing mAbs H7.HK1 and H7.HK2 each fully protected mice without apparent weight loss (FIG. 19, top panels). Given 20 μg per mouse (equivalent to 1 mg/kg), H7.HK2 still fully protected mice from death (defined as >20% weight loss), but with up to 8% average weight loss. H7.HK1 protected 7 out of 10 mice from death, with up to 12% average weight loss for mice that survived (FIG. 19, upper middle panels). By day 2 post challenge, the weight preservation was significantly better in mice receiving 20 μg of H7.HK1 or H7.HK2 than mice receiving the placebo mAb or PBS (FIG. 19, upper middle panels). Mice receiving the non-neutralizing mAbs H7.HK3 or H7.HK4 (100 μg or 20 μg) were not protected and showed no difference from the placebo mAb and PBS controls (FIG. 19, top and upper middle panels).

Without intending to be bound by any theory, in some embodiments, the anti-HA2 mAbs show Fc-mediated function against influenza. The anti-HA2 non-neutralizing mAb H7.HK4 was converted to mouse IgG2a, which mediates strong Fc effector function in mice, and tested for prophylaxis in the mouse challenge model, along with the mouse IgG1 form, which lacks Fc effector function in mice. As shown in FIG. 19 (lower middle panels), given 100 μg per mouse, H7.HK4 mouse IgG2a but not mouse IgG1 protected 4 out of 5 mice from death, with up to 17% average weight loss for mice that survived. By day 3 post challenge, the weight preservation was significantly better in mice receiving H7.HK4 mouse IgG2a than mice receiving H7.HK4 mouse IgG1 or the placebo mouse IgG2a (FIG. 19, lower middle panels). As shown in FIG. 19, (lower middle and upper middle panels), though survived, mice receiving 100 μg H7.HK4 mouse IgG2a lost more weight than those receiving 20 μg neutralizing mAbs H7.HK1 or H7.HK2, indicating less prophylaxis efficiency for H7.HK4 than H7.HK1 and H7.HK2. Since these two mAbs (H7.HK2 and H7.HK4) bind to different sites on the HA and protect through different mechanisms, the combination of a suboptimal dose of 20 μg H7.HK2 (as human IgG1) with 100 μg H7.HK4 mouse IgG2a was tested in the mouse challenge model, using 20 μg H7.HK2 (as human IgG1) with 100 μg H7.HK4 mouse IgG1 as control. As shown in FIG. 19 (bottom panels), compared to this control group, which protected 9 out of 10 mice from death and lost on average up to 11% body weight for mice that survived, the combination of 20 μg of H7.HK2 with 100 μg H7.HK4 mouse IgG2a fully protected mice from death, with only up to 7% average weight loss, and the weight difference was statistically significant between these two groups since day 3 post challenge, indicating a beneficial role of H7.HK4 in the mAb combination regimen. Overlaying the survival and body weight data of the 20 μg H7.HK2 alone group from the previous experiment (FIG. 19), H7.HK2 in combination with H7.HK4 mouse IgG2a did not significantly improve the body weight trough from day 4-6 post challenge as both groups fully protected mice from death with up to 7-8% weight loss; the mAb combination demonstrated a statistical trend and then significance for improved recovery of weight loss starting on day 7 post challenge (FIG. 19).

The experiments demonstrated that the HA2-directed mAb H7.HK4 lacked neutralizing activity but protected mice from a viral challenge when engineered to mouse IgG2a with Fc effector function. Used in combination with H7.HK2 at a suboptimal dose, H7.HK4 augmented the mouse protection. Our data demonstrated a new mechanism of antibody neutralization and an augmented protection against H7N9 when HA1-directed neutralizing mAbs and HA2-directed non-neutralizing mAbs were combined.

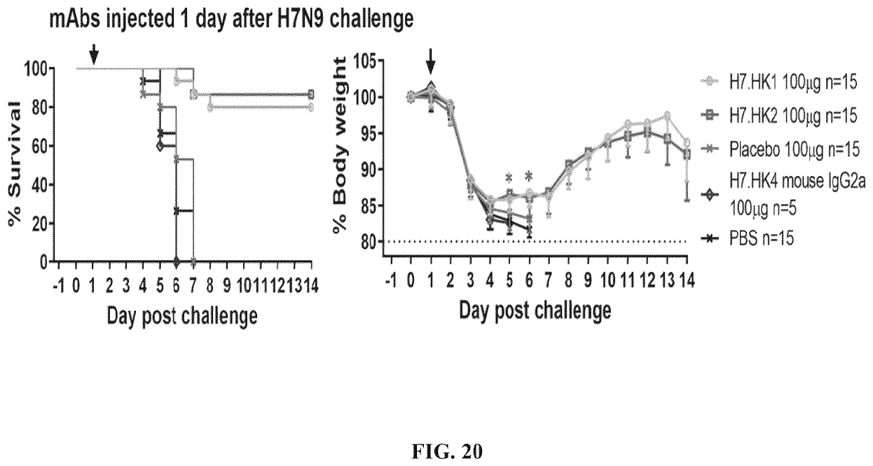

To assess whether the neutralizing mAbs H7.HK1 and H7.HK2 as well as the non-neutralizing mAb H7.HK4 as mouse IgG2a are effective as therapeutics, mice were i.n. challenged (n=5-10 per group from 1-2 experiments) with 10 LD₅₀ of A/Anhui/1/2013 H7N9 virus, not challenged for one day, and then on day 1 post challenge mice were injected i.p. with 100 μg H7.HK1 or H7.HK2 as human IgG1, or H7.HK4 as mouse IgG2a. As shown in FIG. 20, mice were i.p. injected 100 μg of the indicated mAbs one day after viral challenge and the % survival and % body weight of the survived mice were plotted over time. Arrows in FIG. 20 indicate the time when mAbs were administered. Control groups of a non-H7 placebo mAb and PBS were included. Data for each group were combined from 1-2 experiments. Twelve and 13 out of 15 mice receiving 100 μg H7.HK1 or H7.HK2 one day after viral challenge initially lost weight similarly to the placebo and PBS controls but then started to recover on day 5 after challenge. Therefore, the neutralizing mAbs H7.HK1 and H7.HK2 showed both prophylactic and therapeutic efficacies in the mouse challenge model. As shown in FIG. 20, none of the 5 mice receiving 100 μg H7.HK4 mouse IgG2a one day after challenge survived, indicating that the non-neutralizing mAb H7.HK4 as mouse IgG2a demonstrated measurable prophylactic effect but not therapeutic efficacy.

### Example 20: Comparison of H7.HK1/2 to Previously Published Lateral Patch Antibodies

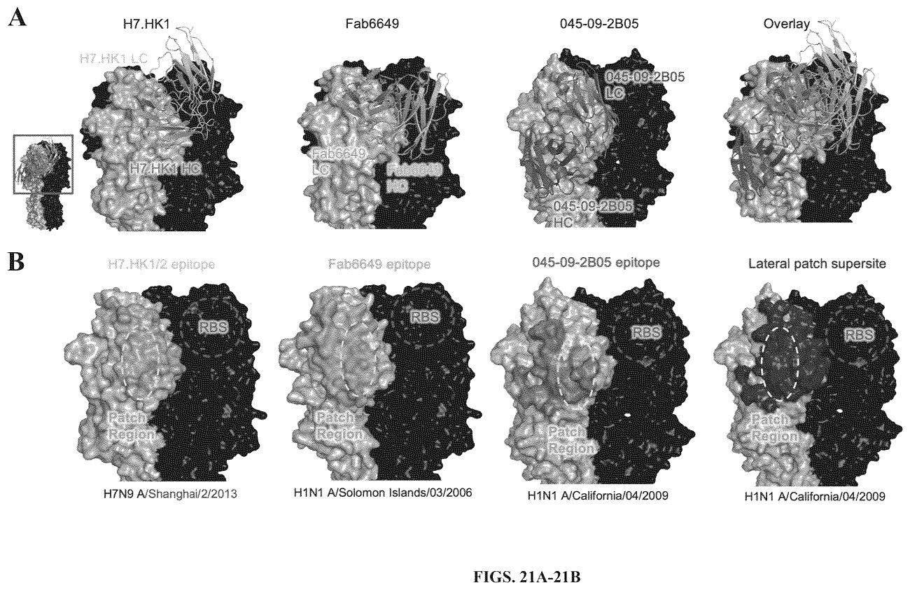

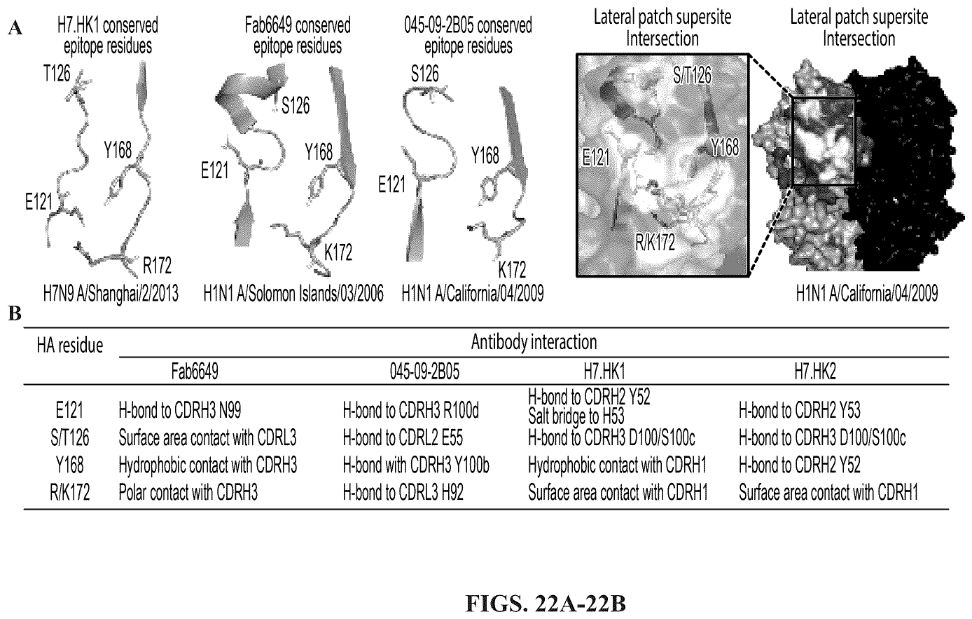

There have been two other published structures of lateral patch-binding antibodies on the HA head, 045-09-2B05 (PDB: 7MEM) and Fab6649 (PDB: 5W6G)—both of which bind H1 (see Raymond D D, Bajic G, Ferdman J, Suphaphiphat P, Settembre E C, Moody M A, Schmidt A G, Harrison S C. 2018. Conserved epitope on influenza-virus hemagglutinin head defined by a vaccine-induced antibody. Proc Natl Acad Sci USA 115:168-173; Guthmiller J J, Han J, Li L, Freyn A W, Liu S T H, Stovicek O, Stamper C T, Dugan H L, Tepora M E, Utset H A, Bitar D J, Hamel N J, Changrob S, Zheng N Y, Huang M, Krammer F, Nachbagauer R, Palese P, Ward A B, Wilson P C. 2021, each of which is hereby incorporated by reference in its entirety). First exposure to the pandemic H1N1 virus induced broadly neutralizing antibodies targeting hemagglutinin head epitopes (See Sci Transl Med 13: eabg4535, incorporated by reference in its entirety). Comparing 045-09-2B05, Fab6649, and H7.HK1 demonstrated diverse angles of approach and different heavy and light chain orientations towards the lateral patch (FIG. 21A). FIG. 21B shows that the epitopes of these antibodies were centered on the lateral patch, with the composite footprint of all three defining a lateral patch supersite of vulnerability. The epitope of H7.HK1 was most similar to that of Fab6649. FIG. 21A-B show that the light chain of H7.HK1 was in a higher position on the head of HA than the heavy chain of Fab6649, which allowed CDR-L1 to clash with the 220-loop. H7.HK1 heavy chain overlapped with the light chain of 045-09-2B05, and the heavy chain of 045-09-2B05 occupied an epitope distinct from H7.HK1, H7.HK2, and Fab6649. FIG. 22A indicates that the epitopes of 045-09-2B05 and Fab6649 had modest overlap, centered around the conserved lateral patch (colored in magenta). Of this overlapping epitope surface, there were four residues conserved between H1 and H7 (positions E121, S/T126, Y168, and R/K172). FIG. 22A shows that the overall structure of this site of vulnerability was also conserved between H1 and H7. Comparison of the interactions between Fab6649, 045-09-2B05, H7.HK1 and these four conserved residues revealed different modes of recognition for each antibody (FIG. 22B). Thus, the lateral patch supersite, as defined by Fab6649, 045-09-2B05, H7.HK1, and H7.HK2, was composed in part by residues that were conserved in H1 and H7 and could be targeted via diverse chemistries and modes of recognition.

Therefore, the epitopes of H7.HK1 and H7.HK2 have been structurally defined to the β14-centered surface of H7 HA1, partially overlapping with antigenic site D, targeting a lateral patch rather than the commonly targeted RBS and trimer interface by previous H7N9 mAbs. Jiao C, Wang B, Chen P, Jiang Y, Liu J. 2023. Analysis of the conserved protective epitopes of hemagglutinin on influenza A viruses. Front Immunol 14:1086297. Structural comparisons demonstrated that H7.HK1 and H7.HK2 interacted with H7 completely differently from L4A-14, H7.167, 07-5F01, and FluA-20. By escape mutations, a previous H3 neutralizing mAb D1-8 was mapped to the lower part of antigenic site D towards site E (Benjamin E, Wang W, McAuliffe J M, Palmer-Hill F J, Kallewaard N L, Chen Z, Suzich J A, Blair W S, Jin H, Zhu Q. 2014. A broadly neutralizing human monoclonal antibody directed against a novel conserved epitope on the influenza virus H3 hemagglutinin globular head. J Virol 88:6743-50, which is hereby incorporated by reference in its entirety.); this epitope partially overlaps with the H7.HK1 and H7.HK2 epitope described here. However, without structural data, the action of neutralization by D1-8 cannot be determined. Importantly, D1-8 does not react to H7, and likewise, H7.HK1 and H7.HK2 do not react to H3. Hence, D1-8 cannot replace the anti-H7N9 function of H7.HK1 and H7.HK2. The unique β14-targeting epitope on the HA1 lateral patch would render H7.HK1 and H7.HK2 favorable candidates for combination prophylaxis and therapy against H7N9 to augment protection efficacy and increase the genetic barrier for viral escape. This is supported by data showing no competition between H7.HK1 and H7.HK2 with two RBS and one non-RBS directed antibodies (FIG. 14D). The surprising results described herein demonstrate that the lateral patch is a viable epitope for H7 vaccine and therapeutic antibody development. Previously reported lateral patch antibodies are all restricted to expressing IGHV3 or IGHV4-39 genes and often had a Y-x-R motif in CDR H3 (see Raymond D D, Bajic G, Ferdman J, Suphaphiphat P, Settembre E C, Moody M A, Schmidt A G, Harrison S C. 2018. Conserved epitope on influenza-virus hemagglutinin head defined by a vaccine-induced antibody. Proc Natl Acad Sci USA 115: 168-173; Guthmiller J J, Han J, Li L, Freyn A W, Liu S T H, Stovicek O, Stamper C T, Dugan H L, Tepora M E, Utset H A, Bitar D J, Hamel N J, Changrob S, Zheng N Y, Huang M, Krammer F, Nachbagauer R, Palese P, Ward A B, Wilson P C. 2021. First exposure to the pandemic H1N1 virus induced broadly neutralizing antibodies targeting hemagglutinin head epitopes. Sci Transl Med 13: eabg4535, each of which is hereby incorporated by reference in its entirety.). H7.HK1 and H7.HK2 are derived from IGHV4-59 and do not contain a Y-x-R motif in CDR H3. Additionally, unlike other reported lateral patch antibodies, H7.HK1 and H7.HK2 disrupt the structure of the 220-loop of the H7 RBS. Therefore, the lateral patch binding site of HA1 is expanded to H7 and composed in part with residues conserved between H1 and H7, which makes it a supersite of vulnerability that could be targeted by more diverse antibodies than previously recognized.

Taken together, the data revealed a new mechanism of antibody neutralization and suggested that the HA1-directed neutralizing mAbs and HA2-directed non-neutralizing mAbs could be used in combination to augment the antiviral effectiveness against H7N9.

### Example 21: Optimization of Antibodies with N-Linked Glycan Removal Mutations

To optimize antibodies, N-linked glycan removal mutants for mAbs H7.HK1, H7.HK2, and H7.HK3 were generated, namely H7.HK1 K_S26A (SEQ ID NO: 1 and SEQ ID NO: 17), H7.HK1 K_N24Q (SEQ ID NO: 1 and SEQ ID NO: 19), H7.HK1 K_N24R (SEQ ID NO: 1 and SEQ ID NO: 21), H7.HK2 K_S28A (SEQ ID NO: 3 and SEQ ID NO: 18), H7.HK2 K_N26Q (SEQ ID NO: 3 and SEQ ID NO: 20), H7.HK2 K_N26S (SEQ ID NO: 3 and SEQ ID NO: 22), and H7.HK3 H_S54A (SEQ ID NO: 23 and SEQ ID NO: 6).

FIG. 23 shows ELISA binding curves of the N-linked glycan removal mutants and their counterpart wild-type antibodies to H7N9 HA1 from the A/Shanghai/2/2013 strain. “K” stands for kappa chain, and “H” stands for heavy chain. The top panel in FIG. 23 shows binding curves of H7.HK1, H7.HK1 K_S26A, H7.HK1 K_N24Q, and H7.HK1 K N24R to H7N9 HA1. The middle panel in FIG. 23 shows binding curves of H7.HK2, H7.HK2 K_S28A, H7.HK2 K_N26Q, and H7.HK2 K_N26S to H7N9 HA1. The bottom panel of FIG. 23 shows binding curves of H7.HK3 and H7.HK3 H S54A to H7N9 HA1. As shown in FIG. 23, all N-linked glycan removal mutants showed similar (top and middle panels) or increased (bottom panel) binding to H7N9 HA1 compared to their wild-type counterparts.

FIG. 24 shows neutralization curves of the N-linked glycan removal mutants and their counterpart wild-type antibodies against H7N9 pseudo virus in MDCK cells. The top panel in FIG. 24 shows neutralization curves of H7.HK1, H7.HK1 K_S26A, H7.HK1 K_N24Q, and H7.HK1 K_N24R against H7N9 pseudo virus. The middle panel in FIG. 24 shows neutralization curves of H7.HK2, H7.HK2 K_S28A, H7.HK2 K_N26Q, and H7.HK2 K N26S against H7N9 pseudo virus. The bottom panel of FIG. 24 shows neutralization curves of H7.HK3 and H7.HK3 H_S54A against H7N9 pseudo virus. As shown in FIG. 24, all N-linked glycan removal mutants neutralized the H7N9 pseudo virus, achieving 100% neutralization at various concentrations. Several of the N-linked glycan removal mutants showed neutralization function comparable to their wild-type counterparts (e.g., H7.HK1 K_N24R in the top panel of FIG. 24). FIG. 24 (bottom panel) also shows that the H7.HK3 H_S54A antibody improved the function of the wild-type mAb H7.HK3, rendering the non-neutralizing H7.HK3 neutralizing against H7N9, with a neutralization IC₅₀ of 0.34 μg/mL as measured by pseudo virus in MDCK cells.

The neutralization curves in FIG. 24 are consistent with results with the ELISA binding curves shown in FIG. 23. For example, H7.HK3 H_S54A (SEQ ID NO: 23 and SEQ ID NO: 6) unexpectedly showed improved binding and improved neutralization compared to H7.HK3. The data in FIGS. 23 and 24 unexpectedly show that, in some embodiments, antibodies containing N-linked glycan removal mutations can effectively bind to H7N9 HA1 and neutralizeH7N9 viruses. These data also unexpectedly show that, in some embodiments, antibodies containing N-linked glycan removal mutations show enhanced binding to H7N9 HA1 and improved neutralization function against H7N9 at lower concentrations compared to their wild-type counterparts (i.e., antibodies without N-linked glycan removal mutations).

The devices, systems, and methods disclosed herein are not to be limited in scope to the specific embodiments described herein. Indeed, various modifications of the devices, systems, and methods in addition to those described will become apparent to those of skill in the art from the foregoing description.

All of the references cited herein are each incorporated by reference herein in their entireties.

What is claimed is:

1. A synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 1, SEQ ID NO: 3, SEQ ID NO: 5, SEQ ID NO: 5, and SEQ ID NO: 7, and the variable light chain domain of the synthetic antibody or fragment thereof comprises an amino acid sequence that is at least 80% identical to SEQ ID NO: 17, SEQ ID NO: 18, SEQ ID NO: 19, SEQ ID NO: 20, SEQ ID NO: 21, or SEQ ID NO: 22.

2. The synthetic antibody or fragment thereof of claim 1, wherein the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 17.

3. The synthetic antibody or fragment thereof of claim 1, wherein the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 19.

4. The synthetic antibody or fragment thereof of claim 1, wherein the variable heavy chain domain of the synthetic antibody or fragment thereof comprises the amino acid sequence that is at least 80% identical to SEQ ID NO: 1, and the variable light chain domain of the synthetic antibody or fragment thereof comprises the amino acid sequence that is at least 80% identical to SEQ ID NO: 21.

5. The synthetic antibody or fragment thereof of claim 1, wherein the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 18.

6. The synthetic antibody or fragment thereof of claim 1, wherein the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 20.

7. The synthetic antibody or fragment thereof of claim 1, wherein the variable heavy chain domain of the synthetic antibody or fragment thereof comprises the amino acid sequence that is at least 80% identical to SEQ ID NO: 3, and the variable light chain domain of the synthetic antibody or fragment thereof comprises the amino acid sequence that is at least 80% identical to SEQ ID NO: 22.

8. The synthetic antibody or fragment thereof of any of claim 1, wherein the antibody or fragment thereof is configured to bind to a hemagglutinin (HA) protein of an H7N9 influenza virus.

9. The synthetic antibody or fragment thereof of claim 1, wherein the antibody or fragment thereof is configured to bind to a region overlapping with an antigenic site D of the globular head domain (HA1) of the H7N9 influenza virus.

10. The synthetic antibody or fragment thereof of claim 9, wherein the antibody or fragment thereof is configured to bind to a β14-centered surface of a lateral patch of the globular head domain (HA1) of the H7N9 influenza virus.

11. The synthetic antibody or fragment thereof claim 10, wherein the antibody or fragment thereof is configured to disrupt a 220-loop of the HA protein required for sialic acid binding.

12. The synthetic antibody or fragment thereof of any of claim 1, wherein the synthetic antibody is configured to neutralize an H7N9 influenza virus.

13. The synthetic antibody of any of claim 1, wherein a structure of the synthetic antibody or fragment thereof is substantially similar to a structure of a reference antibody or fragment thereof, wherein the reference antibody or structure thereof comprises a variable heavy chain domain and a variable light chain domain,
wherein the variable heavy chain domain of the reference antibody or fragment thereof comprises an amino acid sequence selected from the group consisting of SEQ ID NO: 1, SEQ ID NO: 3, SEQ ID NO: 5, and SEQ ID NO: 7, and the variable light chain domain of the reference antibody or fragment thereof comprises an amino acid sequence selected from the group consisting of SEQ ID NO: 17, SEQ ID NO: 18, SEQ ID NO: 19, SEQ ID NO: 20, SEQ ID NO: 21, and SEQ ID NO: 22.

14. A polynucleotide encoding the synthetic antibody of claim 1.

15. A method of preventing or treating an infection in a subject caused by an H7N9 influenza virus, the method comprising administering to the subject an effective amount of a pharmaceutical composition, wherein the pharmaceutical composition comprises the synthetic antibody of claim 1.

16. A synthetic antibody or fragment thereof comprising a variable heavy chain domain and a variable light chain domain, wherein the variable heavy chain domain of the synthetic antibody or fragment thereof comprises an amino acid sequence that is at least 80% identical to SEQ ID NO: 23, and the variable light chain domain of the synthetic antibody or fragment thereof comprises an amino acid sequence that is at least 80% identical to SEQ ID NO: 2, SEQ ID NO: 4, SEQ ID NO: 6, or SEQ ID NO: 8.

17. A vector comprising at least one polynucleotide sequence encoding an antibody or fragment thereof, wherein the antibody or fragment thereof comprises a variable heavy chain domain and a variable light chain domain, wherein the variable heavy chain domain of the antibody or fragment thereof comprises SEQ ID NO: 23, and the variable light chain domain of the antibody or fragment thereof comprises an amino acid sequence selected from the group consisting of SEQ ID NO: 2, SEQ ID NO: 4, SEQ ID NO: 6, and SEQ ID NO: 8.

18. A vector comprising at least one polynucleotide sequence encoding an antibody or fragment thereof, wherein the antibody or fragment thereof comprises a variable heavy chain domain and a variable light chain domain, wherein the variable heavy chain domain of the antibody or fragment thereof comprises an amino acid sequence selected from the group consisting of SEQ ID NO: 1, SEQ ID NO: 3, SEQ ID NO: 5, and SEQ ID NO: 7, and the variable light chain domain of the antibody or fragment thereof comprises an amino acid sequence selected from the group consisting of SEQ ID NO: 17, SEQ ID NO: 18, SEQ ID NO: 19, SEQ ID NO: 20, SEQ ID NO: 21, and SEQ ID NO: 22.

19. A method of making an antibody or fragment thereof comprising:
culturing a host cell under conditions suitable for an expression of a vector,
wherein the vector comprises at least one polynucleotide sequence encoding the antibody or fragment thereof, wherein the antibody or fragment thereof comprises a variable heavy chain domain and a variable light chain domain, wherein the variable heavy chain domain of the antibody or fragment thereof comprises SEQ ID NO: 23, and the variable light chain domain of the antibody or fragment thereof comprises an amino acid sequence selected from the group consisting of SEQ ID NO: 2, SEQ ID NO: 4, SEQ ID NO: 6, and SEQ ID NO: 8; and
recovering the antibody or fragment thereof.

20. A method of preventing or treating an infection in a subject caused by an H7N9 influenza virus, the method comprising administering to the subject an effective amount of a pharmaceutical composition, wherein the pharmaceutical composition comprises:
a synthetic antibody or fragment thereof comprising a variable heavy chain domain and a variable light chain domain, wherein the variable heavy chain domain of the synthetic antibody or fragment thereof comprises an amino acid sequence selected from the group consisting of SEQ ID NO: 1, SEQ ID NO: 3, SEQ ID NO: 5, and SEQ ID NO: 7, and the variable light chain domain of the synthetic antibody or fragment thereof comprises an amino acid sequence selected from the group consisting of SEQ ID NO: 17, SEQ ID NO: 18, SEQ ID NO: 19, SEQ ID NO: 20, SEQ ID NO: 21, and SEQ ID NO: 22.
